(19) United States
(12) Patent Application Publication
(10) Pub. No.: US 2024/0398865 A1
(43) Pub. Date: Dec. 5, 2024

(54) TUMOR AVATAR VACCINE COMPOSITIONS AND USES THEREOF

(71) Applicants: THE BROAD INSTITUTE, INC., Cambridge, MA (US); DANAFARBER CANCER INSTITUTE, INC., Boston, MA (US); THE GENERAL HOSPITAL CORPORATION, Boston, MA (US)

(72) Inventors: Edward Fritsch, Cambridge, MA (US); Nir Hacohen, Cambridge, MA (US); Matthew Bakalar, Cambridge, MA (US)

(73) Assignees: THE BROAD INSTITUTE, INC., Cambridge, MA (US); DANAFARBER CANCER INSTITUTE, INC., Boston, MA (US); THE GENERAL HOSPITAL CORPORATION, Boston, MA (US)

(21) Appl. No.: 18/688,222

(22) PCT Filed: Sep. 1, 2022
(86) PCT No.: PCT/US2022/075817

§ 371 Date: Feb. 29, 2024

Related U.S. Application Data
(60) Provisional application No. 62/239,860, filed on Oct. 10, 2015.

(51) Int. Cl. A61K 35/17; A61K 39/00; A61K 39/125; A61K 45/06; C07K 14/545; C12N 5/0784

(52) U.S. Cl. CPC A61K 35/17; A61K 39/0011; A61K 39/0012; A61K 39/125; A61K 39/4611; A61K 39/4613; A61K 39/4615; A61K 39/46444; A61K 39/464499; A61K 45/06; C07K 14/545; C12N 5/0639; A61K 2039/5152; A61K 2039/53

(57) ABSTRACT

Disclosed herein are methods of eliciting an anti-cancer immune response by administering tumor-associated antigens, cells containing tumor-associated antigens, and/or nucleic acids encoding tumor-associated antigens. inducing immunogenic cell death in the cells expressing or containing the tumor-associated antigens. and optionally generating hyperactivated dendritic cells. Expression of tumor-associated antigens in a separate anatomical site generates a tumor avatar, which mimics the antigenic, but not immunosuppressive, environment of the tumor, with the generation of hyperactivated dendritic cells enhancing antigen presentation to elicit a robust anti-tumor T cell and antibody response. Also provided are compositions and kits containing nucleic acids and other components for use in the methods provided herein.

### RELATED APPLICATIONS

This application claims priority under 35 U.S.C. § 119(e) to U.S. provisional application, U.S. Ser. No. 62/239,860, filed Sep. 1, 2021, which is incorporated by reference herein.

### REFERENCE TO AN ELECTRONIC SEQUENCE LISTING

The contents of the electronic sequence listing (B119570136WO00-SEQ-NTJ.xml; Size: 61,929 bytes; and Date of Creation: Aug. 31, 2022) are incorporated herein by reference.

### BACKGROUND

Cancer is a devastating condition caused by aberrant growth of cells and/or the formation of tumors in a subject, interfering with healthy physiology. Anti-tumor immune responses play an important role in the control of cancer. Specifically, CD8+ T cells specific to tumor antigens (tumor antigens and non-mutated tumor-associated antigens (TAAs)) kill tumor cells following recognition of these antigens presented by molecules of the major histocompatibility complex (MHC) on the tumor cell surface, while surface molecules signaling cell stress and antibodies bound to surface tumor antigens mark tumor cells for killing by natural killer cells. However, the tumor microenvironment is often characterized by immunosuppressive conditions, such as the presence of myeloid-derived suppressor cells, regulatory T cells, PD-L1 expression on tumor cells and some immune cells, and the presence of anti-inflammatory cytokines, which hinder the generation and function of tumor-specific immune responses.

### SUMMARY

Provided herein are systems, methods, compositions, and kits for inducing an anti-tumor immune response in a subject by generating a population of cells containing one or more tumor antigens, known as a “tumor avatar,” at a vaccination site. The activation of T cells specific to tumor antigens requires the presentation of peptides found in tumor cells by antigen-presenting cells which have internalized all or part of the tumor cell and processed the proteins from those cancer cells into peptides which can be presented on the surface of the APC by MHC molecules. However, the tumor microenvironment is often immunosuppressive, containing inhibitory cells (e.g., myeloid-derived suppressor cells, regulatory T cells, and PD-L1+ cells) and anti-inflammatory cytokines, which hinder the generation of an effective immune response to tumor antigens. Tumor avatars provide an additional source of tumor antigens in an alternative anatomical site to avoid the immunosuppressive effects of the tumor microenvironment. To generate a tumor avatar, nucleic acids encoding tumor antigens, tumor antigen-containing cells, or proteins that both comprise tumor antigens themselves and have a receptor binding domain that target those proteins to local engineered ‘avatar’ cells are administered at an injection site, such as the skin or muscle, that is separate from the location of a tumor. Avatar cells are then induced to undergo immunogenic cell death by administration of chemicals or other therapies. Local immune modulators, such as inflammatory cytokines or activating ligands, are also administered to promote tumor antigen uptake and presentation. Additionally, chemical or mechanical treatments, such as photodynamic therapy, ultrasound, and oxidation of phospholipids at the injection site may be used to further enhance the immune response. By establishing a tumor avatar to provide an alternative cellular source of antigens away from the immunosuppressive environment of the tumor, this approach elicits an effective anti-tumor response, leading to improved outcomes in the treatment of cancer.

Accordingly, the present disclosure provides, in some aspects, a method of eliciting an immune response to a tumor antigen in a subject, the method comprising:
(a) administering to the subject a tumor antigen, a nucleic acid encoding a tumor antigen, or a tumor antigen-containing cell at a site that is separate from a tumor in the subject, whereby the administering generates a tumor avatar in the subject at a site that is separate from the tumor in the subject, wherein the tumor avatar comprises a population of tumor antigen-containing cells; and
(b) causing death of one or more cells of the tumor avatar, whereby cell death releases the tumor antigen and stimulates an inflammatory response; and
whereby an immune response specific to the tumor antigen is generated in the subject. Tumor antigen-containing cells may be generated by administering a nucleic acid encoding a tumor antigen, which is then expressed in cells; by administering a tumor antigen directly, which is taken up by cells in the subject; or by introducing a tumor antigen or nucleic acid encoding the same to cells in vitro or ex vivo, followed by administering the tumor antigen-containing cells to the subject.

In some embodiments, the method further comprises:
(c) generating hyperactivated dendritic cells in the subject, wherein the hyperactivated dendritic cells secrete IL-1β and/or IL-18. Hyperactivated dendritic cells more readily migrate to the draining lymph node, where they present antigens to T cells to activate T cells specific to tumor antigens. Secretion of IL-1β and IL-18, without the cell death (pyroptosis) that is usually associated with IL-1β secretion, promotes inflammation and antigen uptake, consequently improving the generation of tumor antigen-specific CD8+ T cells. Pyroptotic dendritic cells secrete large amounts of IL-1β, but are killed in the process, and are thus unable to present antigens to T cells. By contrast, hyperactivated dendritic cells secrete IL-1β while still being viable and able to present antigens, and are thus more effective at generating a robust anti-tumor T cell response. In some embodiments, Fms-related tyrosine kinase 3 ligand (FLT3L) is administered to promote mobilization of dendritic cells.

In some embodiments, hyperactivated dendritic cells are generated by exposure to a hyperactivating ligand. In some embodiments, the hyperactivating agent is an oxidized phospholipid, a LysM-containing protein, an HMGB1, a granzyme, an IL-1β-containing protein, a Toll-like receptor-stimulating protein, a virus, or a virus-like particle. Each of these hyperactivating ligands stimulate a cell surface receptor, such as a Toll-like receptor, that results in caspase activation and inflammasome-mediated IL-1β secretion, or activates a caspase or inflammasome directly. Each of these and other hyperactivating agents are described in more detail below.

Some aspects of the present disclosure relate to causing the death of one or more cells of the tumor avatar. Cell death releases tumor antigens from tumor antigen-containing cells, as well as components of any dead cells of the tumor avatar, including healthy cells near the tumor antigen-containing cells, which trigger the secretion of inflammatory cytokines and chemokines, as well as the recruitment of innate immune cells, such as monocytes, macrophages, neutrophils, and dendritic cells. Immunogenic cell death is also a source of antigens for antigen presenting cells. Immunogenic cell death of tumor antigen-containing cells separate from a tumor site in the subject thus promotes the generation of T cells and antibodies specific to the introduced tumor antigens outside of an immunosuppressive tumor, which can then result in an effective anti-tumor response. In some embodiments, cell death is caused by administering a cytotoxic agent to the subject, by administering a virus to the subject, or by administering a sensitizing agent to the subject and exposing the sensitizing agent to an energy source.

Each of the compositions used in the methods provided herein, including cells, nucleic acids, and antigens, may be administered by any of method of administration, including injection (e.g., jet injection or hydrodynamic injection), skin scarification, and/or in vivo electroporation. A composition may be administered by any of multiple routes, including intramuscularly, intradermally, or subcutaneously.

In some embodiments, a tumor avatar is generated by administering to the subject a nucleic acid library encoding tumor antigen(s). The nucleic acids of the library may encode any number of tumor antigens, up to an including all tumor antigens or all proteins being expressed in a tumor or tumor cell of the subject. The nucleic acid library may contain nucleic acids, such as DNAs (e.g., plasmids, minicircles, artificial chromosomes) and/or RNAs (e.g., mRNAs). A tumor in a subject may be sequenced to determine which antigens are being expressed, to inform the design of the library and ensure inclusion of any personal tumor antigens that are unique to the subject. A library can also be prepared based on the expressed RNAs in the cell and without sequencing. Additionally, one or more shared tumor antigens, such as those described in The Cancer Genome Atlas (http://cancergenome.nih.gov/), may be encoded by a nucleic acid of the library.

In some embodiments, the methods provided herein result in the generation of an immune response to a tumor antigen in the subject. Such an immune response may be characterized by the induction of one or more inflammatory cytokines, such as IL-1β and IFN-γ, the generation of T cells specific to tumor antigens, and/or the generation of B cells that produce antibodies specific to tumor antigens.

In some embodiments, the methods provided herein reduce the mass and/or volume of a tumor in the subject, reduce the number of cancerous cells in a tumor, reduce the number of circulating cancer cells in the subject, prevent or reduce metastasis in the subject, and/or prevent the recurrence of cancer in a subject.

In some embodiments of the methods provided herein, the method further comprises administering one or more anti-cancer agents, such as an immune checkpoint inhibitor or chemotherapeutic agent, to the subject.

In some aspects, the present disclosure relates to fusion proteins comprising an anchoring domain and a cytokine domain. A protein comprising an anchoring domain is more likely to remain in a local anatomical site, such as the tumor avatar where it was produced, and exert a biological effect in that site rather than diffusing or circulating elsewhere in the body. By joining the anchoring domain with a cytokine domain, the resulting fusion protein can be retained in a desired anatomical site, allowing the cytokine domain to interact with cytokine receptors of nearby cells.

In some aspects, the present disclosure relates to engineered receptors to be expressed on the surface of cells in the subject, the engineered receptor comprising an intracellular domain of CLEC9A; a transmembrane domain; and an extracellular domain that is capable of binding to a tumor antigen, such as by adhering to a ligand that is included on a tumor antigen. When expressed on cells of the subject, the engineered receptor binds extracellular tumor antigens, retaining them in the same anatomical site, where they can be taken up by dendritic cells for presentation, as opposed to circulating elsewhere in the body.

Also provided herein are compositions and kits including tumor antigens, nucleic acids encoding tumor antigens, tumor antigen-containing cells, hyperactivating agents, hyperactivated dendritic cells, engineered receptors, fusion proteins, and/or delivery devices, for use in treating cancer.

Non-limiting examples of cancers that may be treated by any of the methods, systems, compositions, and kits provided herein include melanoma, squamous cell cancer, small-cell lung cancer, non-small cell lung cancer, lung cell adenocarcinoma, squamous lung cell carcinoma, peritoneal cancer, hepatocellular cancer, gastrointestinal cancer, esophageal cancer, stomach cancer, pancreatic cancer, glioblastoma, cervical cancer, ovarian cancer, liver cancer, bladder cancer, hepatoma, breast cancer, colon cancer, colorectal cancer, endometrial carcinoma, uterine carcinoma, salivary gland carcinoma, kidney cancer, prostate cancer, vulval cancer, thyroid cancer, hepatic carcinoma, gastric cancer, head-and-neck cancer, leukemia, and lymphoma. In some embodiments, a method, system, composition, or kit is provided herein for treating a solid tumor. In some embodiments, a method, system, composition, or kit is provided herein for treating a hematologic malignancy.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1C shows methods of generating tumor avatars in a subject to generate anti-tumor immunity. FIG. 1A shows the characteristics of tumor avatars (left), containing hyperactivated dendritic cells and healthy cells expressing tumor antigens, which are induced to die in an immunogenic manner, and tumors (right), which contain tumor cells expressing immunosuppressive PD-L1, myeloid-derived suppressor cells (MDSCs), T regulatory (Treg) cells, and M2 tumor-associated macrophages (M2-TAMs). FIG. 1B shows the generation of tumor avatars by transducing nucleic acids encoding tumor antigens into cells of a subject, and introducing a hyperstimulating polypeptide or exposing the subject to an energy source to cause cell death. Death of tumor antigen-containing cells releases oxidized phospholipids and/or other molecules that signal cell damage, resulting in dendritic cell hyperactivation, as well as tumor antigens, which can be internalized and presented by dendritic cells to activate CD8+ and CD4+ T cells. FIG. 1C shows an exemplary method of generating tumor avatars by delivering modified vaccinia Ankara (MVA) virus by skin scarification and delivery of nucleic acids encoding tumor antigens.

FIGS. 2A-2B show the natural cycle of the immune response to cancer. FIG. 2A shows the steps of cancer cell death, antigen-presenting cell migration, antigen presentation, T cell activation, and T cell-mediated killing of cancer cells. FIG. 2B shows, for each step in the cycle of FIG. 2A, the immunostimulatory factors (top group) and inhibitory factors (bottom group).

FIG. 3 shows the phenotype of hyperactivated dendritic cells, which exhibit robust antigen uptake, antigen presentation, costimulation, migration, and IL-1β secretion. Adapted from Zhivaki et al. 2020. Cell Reports. 33(7):108381.

FIG. 4 shows the generation of artificial Adjuvant Vector Cells (aAVCs) carrying a tumor antigen and CD1d bound to NK-T cell ligand alphaGalactosyl Ceramide (αGC), which stimulate maturation of dendritic cells in vivo, presentation of the tumor antigen, and generation of a robust CD4+ and CD8+ T cell response to the tumor antigen. Adapted from Fuji et al., 2022. Cancer Sci. 113(3):864-874.

### DEFINITIONS

A “tumor avatar,” as used herein, refers to a population of cells, each cell engineered to contain one or more tumor antigens, or a population of tumor antigens, in a subject that are located in an anatomical site that is separate from a tumor in the subject. Cells of the tumor avatar may be engineered to contain a tumor antigen by the administration of one or more nucleic acids encoding a tumor antigen, administration of a tumor antigen which is taken up by a cell in a subject, or by the administration of tumor antigen-containing cells to a subject at a site that is separate from a tumor in the subject.

A “tumor antigen,” as used herein, refers to a protein that is present in a tumor, or comprises an amino acid sequence that is present in a tumor. A tumor antigen may be a full-length form of a protein that is present in a tumor or a peptide fragment of a protein that is present in a tumor. A “tumor-associated antigen” refers to a self-antigen that is expressed by tumor cells at a higher level than healthy cells of a subject. A “tumor-specific antigen” refers to an antigen that is expressed by tumor cells, but not non-tumor cells. A tumor-specific antigen may be a “neoantigen,” which contains a novel amino acid sequence due to one or more mutations.

A “dendritic cell,” as used herein, refers to a cell of the myeloid lineage that is capable of presenting peptides on major histocompatibility complex (MHC) class II and class I proteins, and comprises one or more extensions, or dendrites, that increase the surface: volume ratio of the cell relative to a cell without such an extension. Dendritic cells are capable of activating CD4+ T helper cells by presenting peptides on MHC-II, and of activating CD8+ cytotoxic T lymphocytes by presenting peptides on MHC-I. Thus, dendritic cells are one of the primary antigen-presenting cells that bridge the innate and adaptive immune systems. See, e.g., Borst et al., Nat Rev Immunol. 2018. 18(10):635-647 and Sánchez-Paulete et al., Ann Oncol. 2017. 28(s12):xii44-xii55.

A “nucleic acid,” or “polynucleotide,” as used herein, refers to an organic molecule comprising two or more covalently bonded nucleotides. A “nucleotide,” as used herein, refers to an organic molecule comprising a 1) a nucleoside comprising a sugar covalently bonded to a nitrogenous base (nucleobase); and 2) a phosphate group that is covalently bonded to the sugar of the nucleoside. Nucleotides in a polynucleotide are typically joined by a phosphodiester bond, in which the 3′ carbon of the sugar of a first nucleotide is linked to the 5′ carbon of the sugar of a second nucleic acid by a bridging phosphate group. Typically, the bridging phosphate comprises two non-bridging oxygen atoms, which are bonded only to a phosphorus atom of the phosphate, and two bridging oxygen atoms, each of which connects the phosphorus atom to either the 3′ carbon of the first nucleotide or the 5′ carbon of the second nucleotide. In a nucleic acid sequence describing the order of nucleotides in a nucleic acid, a first nucleotide is said to be 5′ to (upstream of) a second nucleotide if the 3′ carbon of first nucleotide is connected to the 5′ carbon of the second nucleotide. Similarly, a second nucleotide is said to be 3′ to (downstream of) a first nucleotide if the 5′ carbon of the second nucleotide is connected to the 3′ carbon of the first nucleotide. Nucleic acid sequences are typically read in 5′->3′ order, starting with the 5′ nucleotide and ending with the 3′ nucleotide.

A “hyperactivating agent,” as used herein, refers to a molecule that activates a response by a cell of the immune system resulting in the generation of hyperactivated dendritic cells.

A “virus,” as used herein, refers to an obligate intracellular parasite that comprises at least a nucleic acid genome and one or more proteins. A virus particle, or virion, replicates and produces more virus particles by 1) attaching to a cell, 2) delivering the genome into a site inside the cell, such as the cytoplasm or nucleus, 3) producing one or more viral proteins and synthesizing copies of the genome, and 4) packaging newly synthesized viral genomes into new viral particles. See, e.g., Fields, B. N., Knipe, D. M., Howley, P. M., & Griffin, D. E. (2001). Fields virology. Philadelphia: Lippincott Williams & Wilkins. A “virus-like particle,” as used herein, refers to a particle containing one or more viral proteins, but that is not capable of replication, due to lack of a full viral genome or complete absence of the viral genome.

An “inflammatory cytokine,” as used herein, refers to a cytokine that promotes one or more responses associated with inflammation.

An “activating ligand,” as used herein, refers to a molecule that binds to a cell surface receptor, causing transduction of an immunostimulatory signal to the cell. Non-limiting examples of immunostimulatory signals include cytokine secretion, antibody secretion, modulation of surface receptor expression, and modulation of metabolic pathways. Non-limiting examples of activating ligands include the ligands of inhibitory receptors and the ligands of activating receptors

As used herein, “antibody” refers to a polypeptide of the immunoglobulin family that is capable of binding a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

A “sensitizing agent,” as used herein, refers to a composition that increases the sensitivity of an anatomical site (e.g., skin, muscle, epithelium) containing the sensitizing to an energy source. As used herein, a “photosensitizing agent” is a composition or molecule that increases the sensitivity of an anatomical site in a subject, such as the skin, to a light source. As used herein, an “acoustic sensitizing agent” is a composition or molecule that increases the sensitivity of an anatomical site in a subject, such as the skin, to a source of sound.

The terms “identical” and its grammatical equivalents as used herein or “sequence identity” in the context of two nucleic acid sequences or amino acid sequences of polypeptides refers to the residues in the two sequences which are the same when aligned for maximum correspondence over a specified comparison window. An exemplary “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci. U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. In one class of embodiments, the polypeptides herein have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can have 50%, 60%, 70%, 75%, 80%, 85%, 90%, 98%, 99%, or 100% sequence identity to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

“Substantially identical” and its grammatical equivalents as applied to nucleic acid or amino acid sequences mean that a nucleic acid or amino acid sequence comprises a sequence that has at least 90% sequence identity or more, at least 95%, at least 98% and at least 99%, compared to a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In embodiments, the substantial identity exists over a region of the sequences that is at least about 50 residues in length, over a region of at least about 100 residues, and in embodiments, the sequences are substantially identical over at least about 150 residues. In embodiments, the sequences are substantially identical over the entire length of the coding regions.

### DETAILED DESCRIPTION

The present disclosure provides, in some aspects, systems, compositions, and methods of generating an anti-tumor immune response in a subject by generating a “tumor avatar” in the subject to provide an alternative source of tumor antigens away from the immunosuppressive environment of the tumor. An effective anti-tumor immune response requires the activation and proliferation of T cells, such as CD8+ T cells, that are specific to antigens present specifically in the tumor, such as tumor antigens produced by cancerous cells due to mutations. However, the tumor microenvironment is often immunosuppressive, containing anti-inflammatory cytokines and suppressor cells that hinder multiple aspects of the presentation of tumor antigens to T cells and the generation of a robust anti-tumor CD8+ T cell response. Such immunosuppressive mechanisms may also be found in the lymphatic structures that drain the tissue where tumors are located. To bypass the immunosuppressive environment of the tumor and facilitate activation of CD8+ T cells, a tumor avatar is generated in a subject by administering one or more tumor antigens capable of binding to and/or being internalized by a cell, nucleic acids encoding tumor antigens, or tumor antigen-containing cells at an anatomical site that is distant from a tumor in the subject, serving as an alternative source of tumor antigens. For example, in a subject with a liver tumor, the tumor avatar may be generated in an arm. Tumor antigens can be delivered to the subject and introduced into the intracellular space or stably bound to the surface of the cell. Following immunogenic cell death that results in dendritic cell recruitment and activation, dendritic cells may acquire tumor antigens directly from the intracellular space or cell surface. Alternatively, tumor antigens may be delivered into cells of the subject, such as by a lipid nanoparticle carrier. Nucleic acids may also delivered to the subject for introduction into cells, such through a viral vector or lipid nanoparticle, after which they are expressed to produce tumor antigens. Finally, cells containing tumor antigens may be generated ex vivo or in vitro, and administered to the subject. Tumor antigen-containing cells generated in vivo, tumor antigen-containing cells that are directly administered, and/or healthy cells near the directly administered tumor antigens are killed in an immunogenic manner, such as by administration of a virus or exposure to an energy source, which releases tumor antigens at the site of the tumor avatar. Released tumor antigens are taken up by nearby dendritic cells, which present antigens in the draining lymph node to activate CD4+ and CD8+ T cells specific to the tumor antigens. Because the environment of the tumor avatar is not immunosuppressive like that of a tumor, tumor antigen uptake and presentation is more efficient, leading to the generation of a more effective anti-tumor immune response. Additionally, dendritic cells near the site of the tumor avatar may be hyperactivated by the release of oxidized phospholipids from dying cells, or the administration of a hyperactivating agent. Hyperactivated dendritic cells, characterized by IL-1β and IL-18 secretion, are especially effective at presenting peptides to T cells, and so facilitate the activation of CD4+ and CD8+ T cells specific to the tumor antigens of the tumor avatar. The generation of a tumor avatar by the administration of nucleic acids encoding tumor antigens and the induction of hyperactivated dendritic cells thus allows for the generation of an effective anti-tumor immune response in a subject. Also provided are compositions comprising 1) tumor antigens, nucleic acids encoding tumor antigens, and/or tumor antigen-containing cells for generating tumor avatars in a subject at a site that is separate from a tumor in a subject; and 2) hyperactivating agents for generating hyperactivated dendritic cells that secrete IL-1β and IL-18 in a subject. Tumor antigens, whether encoded by nucleic acids, provided as proteins themselves, or within tumor antigen-containing cells, may be administered to the subject separately from the hyperactivating agent. Further provided are kits comprising compositions for generating tumor avatars and hyperactivating dendritic cells in a subject, and cytotoxic or sensitizing agents for the induction of immunogenic cell death in a subject, to facilitate the release of tumor antigens and presentation of tumor antigens to CD8+ T cells. Finally, the present disclosure provides engineered proteins for inducing an inflammatory or anti-tumor immune response in a subject. In some embodiments, an engineered protein is a fusion protein comprising an anchoring domain, to localize the fusion protein at or near the tumor avatar, and a cytokine domain, to stimulate cytokine receptors on cells in or near the tumor avatar. In other embodiments, an engineered protein is an engineered receptor comprising an extracellular domain that is capable of binding to a tumor antigen fused to a ligand for the engineered receptor, to retain tumor antigens near the tumor avatar, and an intracellular domain of CLEC9A, which facilitates the trafficking of proteins for processing and cross-presentation to CD8+ T cells. Expression of an anti-CLEC9A antibody, CLEC9A-binding protein, or peptide-binding domain on the surface of a cell in or near the tumor avatar facilitates adherence of CLEC9A+ cells, such as CLEC9A+ dendritic cells, thereby promoting antigen uptake and presentation.

### Generation of Tumor Avatars

Aspects of the present disclosure relate to methods of administering tumor antigens, nucleic acids encoding tumor antigens, and/or cells containing tumor antigens (tumor antigen-containing cells) to a subject at a site that is separate from a tumor in the subject, such as the skin or an extremity. Tumor antigens may be tumor-associated antigens, which are expressed at higher levels in tumor cells than human cells, or tumor-specific antigens, which are not expressed in healthy cells. Tumor-specific antigens may be neoantigens. Neoantigens, which arise as a result of genetic change (e.g., inversions, translocations, deletions, missense mutations, splice site mutations, etc.) within malignant cells, represent the most tumor-specific class of antigens and can be subject-specific (personal) or detected in more than one subject (shared). Neoantigens are specific to the tumor as the mutation and its corresponding protein are present only in the tumor. Neoantigens also avoid central tolerance, as they are not expressed in the thymus for negative selection of tumor antigen-specific T cells, and are therefore more likely to be immunogenic. Neoantigens thus provide an excellent target for immune recognition including by both humoral and cellular immunity. However, the tumor microenvironment is often immunosuppressive, which hinders the generation of an effective immune response to tumor antigens such as neoantigens.

When administered to a subject, nucleic acids encoding a tumor antigen can result in production of the tumor antigen in the cells of a subject. Expression of tumor antigens in a subject, or introduction of tumor antigens or tumor antigen-containing cells, allows them to be presented to cells of the adaptive immune system, such as B cells and T cells, to activate B and T cells that recognize the tumor antigens, and initiate an anti-tumor immune response. Administration of nucleic acids encoding tumor antigens at a site separate from a tumor results in the production of a tumor avatar, or a collection of cells that, because it contains multiple, such as 2-50 or more, tumor antigens, mimics the antigenic composition of the tumor. Similarly, administration of tumor antigens or a tumor antigen-containing cell at a site that is separate from a tumor in the subject generates a secondary source of tumor antigens that is outside the immunosuppressive environment of the tumor. This secondary source of tumor antigens is referred to as a “tumor avatar,” which is defined herein as a collection of tumor antigen-containing cells that is physically separate from a tumor in the subject. The tumor antigen-containing cells of the tumor avatar may be directly administered to the subject, or produced by the administration of a tumor antigens or nucleic acid encoding a tumor antigen to the subject. In some embodiments, the tumor antigen-containing cells are separated from a tumor in the subject by a distance of at least 1 cm-2 m. In some embodiments, the tumor antigen-containing cells are separated from a tumor in the subject by a distance of at least 1 cm, at least 2 cm, at least 3 cm, at least 4 cm, at least 5 cm, at least 6 cm, at least 7 cm, at least 8 cm, at least 9 cm, at least 10 cm, at least 15 cm, at least 20 cm, at least 25 cm, at least 30 cm, at least 35 cm, at least 45 cm, at least 50 cm, at least 60 cm, at least 70 cm, at least 80 cm, at least 90 cm, or at least 100 cm. In some embodiments, the tumor avatar is generated in an organ that is different from the organ where a tumor is located in the subject. In some embodiments, the tumor avatar is generated in an organ that is different from where a metastasis is detected in the subject.

Tumor avatars produced by the methods provided herein resemble the antigenic environment of the tumor, as cells express tumor antigens encoded by the administered nucleic acids, but, importantly, do not mimic the immunosuppressive environment that often develops in and around tumors. For example, tumor cells may express checkpoint inhibitors such as PD-L1, which, upon binding to PD-1 on the surface of T cells, transmit an inhibitory signal to the T cell. Additionally, the tumor microenvironment is often rich in myeloid-derived suppressor cells (MDSCs), M2 tumor-associated macrophages (M2-TAMs), and T regulatory (Treg) cells. Each of these cell types exerts immunosuppressive effects in the tumor, such as secreting anti-inflammatory cytokines (e.g., IL-10 and TGF-β), transmitting inhibitory signals to T cells, competing with other anti-cancer T cells for limited amounts of pro-survival cytokines (e.g., IL-2). See, e.g., Labani-Motlagh et al., Front Immunol. 2020. 11:940. Furthermore, Treg cells specifically can reprogram dendritic cells to induce an anti-inflammatory, rather than a pro-inflammatory, response by T cells following antigen presentation. See, e.g., Alpan et al., Nat Immunol. 2004. 5(6):615-622. Unlike the tumor microenvironment, which often contains many immunosuppressive cytokines that interfere with the generation of an immune response, the tumor avatar promotes the generation of an immune response to tumor antigens. In addition to containing tumor-associated antigens, the tumor avatar can be further modified to a pro-inflammatory state that facilitates the recruitment of immune cells, antigen uptake, antigen presentation, and the activation of anti-tumor CD4+ and CD8+ T cells. An inflammatory response is induced in the tumor avatar by causing the death of one or more cells of the tumor avatar. Cell death releases tumor antigens from tumor antigen-containing cells, as well as components of any dead cells of the tumor avatar, including healthy cells near the tumor antigen-containing cells, which trigger the secretion of inflammatory cytokines and chemokines, as well as the recruitment of innate immune cells, such as monocytes, macrophages, neutrophils, and dendritic cells. Immunogenic cell death is also a source of antigens for antigen presenting cells. Immunogenic cell death of tumor antigen-containing cells separate from a tumor site in the subject thus promotes the generation of T cells and antibodies specific to the introduced tumor antigens outside of an immunosuppressive tumor, which can then result in an effective anti-tumor response.

In some embodiments, a tumor antigen, nucleic acid encoding a tumor antigen, and/or tumor antigen-containing cell is administered to the subject at a site that is separate from a tumor in the subject.

In some embodiments of the systems, methods, compositions, uses, and kits provided herein, the nucleic acid encoding the tumor antigen, or the nucleic acids of the nucleic acid library, are DNAs. In some embodiments, one or more DNAs is a plasmid, minicircle, or artificial chromosome. In some embodiments of the systems, methods, compositions, uses, and kits provided herein, the nucleic acid encoding the tumor antigen, or the nucleic acids of the nucleic acid library, are RNAs. In some embodiments, one or more nucleic acids is an mRNA. An mRNA, or messenger RNA, is a polynucleotide comprising an open reading frame that is capable of being translated into a protein. An open reading frame (ORF) refers to a nucleic acid sequence that can be translated into a protein by cellular components, such as ribosomes and aminoacyl-tRNAs. Generally, an open reading begins with a START codon, such as AUG (RNA) or ATG (DNA), and ends with a STOP codon, such as UAG, UAA, or UGA (RNA) or TAG, TAA, or TGA (RNA), with the number of bases between the last nucleotide of the START codon and the first nucleotide of the STOP codon being a multiple of 3 (e.g., 3, 6, 9, etc.). An mRNA may also comprise a 5′ untranslated region (5′ UTR) that is 5′ to (upstream from) the open reading frame, a 5′ cap that is covalently bonded to the first nucleotide of the mRNA, a 3′ UTR that is 3′ to (downstream from) the open reading frame, and/or a poly(A) tail that is 3′ to (downstream from). A poly(A) tail comprises a sequence of consecutive adenosine nucleotides, which protect the mRNA from degradation by 3′ exonucleases. In some embodiments, the poly(A) tail comprises 25-500 nucleotides. In some embodiments, the poly(A) tail comprises 50-300 nucleotides.

In some embodiments of the nucleic acids provided herein, the nucleic acid comprises at least one modified nucleotide. A modified nucleotide may comprise a modified nucleobase, a modified sugar, and/or a modified phosphate. In some embodiments, at least one modified nucleotide comprises a modified nucleobase selected from the group consisting of: xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl) propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). In some embodiments, at least one modified nucleotide comprises a modified sugar selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose. In some embodiments, at least one modified nucleotide comprises a modified phosphate selected from the group consisting of phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate. In some embodiments, a nucleic acid comprises more than one modified nucleotide. In some embodiments, at least 1%, 2%, 3%, 4%, 5%, 6%, 7%, 8%, 9%, or 10% of the nucleotides of the nucleic acid are modified nucleotides. RNA encoding tumor antigens or hyperactivating agents or other immune stimulators may be delivered in multiple forms, such as linear RNA, branched RNA, or circular RNA.

In some embodiments of the systems, methods, compositions, uses, and kits provided herein, the nucleic acid encoding the tumor antigen, or the nucleic acids of the nucleic acid library, are DNAs. A DNA is a polynucleotide comprising multiple covalently bonded deoxyribonucleotides. Unlike an mRNA, a DNA encoding a tumor antigen is not immediately translated into a protein by cellular ribosomes and aminoacyl-tRNAs. Instead, the administered DNA is first transcribed into a precursor mRNA (pre-mRNA) by an RNA polymerase, then the pre-mRNA is processed into a mature RNA by (optionally) RNA splicing, addition of a poly(A) tail, and addition of a 5′ cap. In some cases, the mature RNA is a circular RNA. The mature mRNA is then exported from the nucleus and translated into the encoded protein. The DNA may comprise one or more regulatory elements, such as a promoter, upstream of the nucleic acid sequence encoding the tumor antigen to control transcription. In some embodiments, the promoter is operably linked to the nucleic acid sequence encoding the tumor antigen. A promoter is said to be operably linked to a nucleic acid sequence if an RNA polymerase is capable of binding to the promoter and initiating transcription of the DNA sequence to produce an RNA that is complementary to the DNA sequence. In some embodiments, the promoter is a constitutive promoter. A constitutive promoter mediates transcription of an operably linked nucleic acid sequence at a consistent rate in a cell. In some embodiments, the promoter is an inducible promoter. An inducible promoter mediates transcription of an operably linked nucleic acid sequence at a level that is sensitive to one or more environmental conditions, such as the presence or absence of an input molecule. DNAs encoding tumor antigens and/or hyperactivating agents may be delivered in multiple forms, such as linear DNA fragments, plasmids, minicircles, and artificial chromosomes. DNAs may also be delivered by a viral vector, which infect the cell and deliver the DNA, where it can be transcribed. Non-limiting examples of viral vectors include adenoviruses, adeno-associated viruses, herpesviruses, lentiviruses, retroviruses, and poxviruses.

In some embodiments of the systems, methods, compositions, uses, and kits provided herein, the administered tumor antigen(s), tumor antigen(s) encoded by the nucleic acid or nucleic acids of the nucleic acid library, and/or tumor antigens in tumor antigen-containing cells are proteins that are present in the subject. In some embodiments, a nucleic acid sequence encoding the tumor antigen is a nucleic acid sequence that is present on a nucleic acid in the subject. Methods of determining whether a protein or nucleic acid is present in a subject are known in the art, and generally include obtaining a biological sample from the subject and analyzing the contents of the sample. A biological sample may be obtained from a subject by one or more of multiple methods, such as a biopsy or blood draw. The contents of a biological sample may be analyzed by one or more of multiple methods, such as proteomics, western blot, PCR, qRT-PCR, DNASeq, or RNAseq.

In some embodiments of the systems, methods, compositions, uses, and kits provided herein, the tumor antigen(s) of the tumor avatar are also present in a tumor in the subject. In some embodiments, a nucleic acid encoding a tumor antigen encoded by the administered nucleic acid or a nucleic acid of the administered nucleic acid is present in the subject. Thus, an immune response specific to an antigen that is present in a tumor in the subject is generated. Methods of determining whether a protein or nucleic acid are present in a tumor in a subject are similar to those described in the preceding paragraph, with the biological sample being obtained from a tumor in the subject, such as by a biopsy.

In some embodiments of the systems, methods, compositions, uses, and kits provided herein, a tumor antigen of the tumor avatar is not present in healthy cells in the subject. In some embodiments, the tumor antigen is present at lower levels in healthy cells than in tumor cells of the subject. In some embodiments of the methods, compositions, and kits provided herein, the tumor antigens of the tumor avatar are not present in the subject, wherein the generation of an immune response to the tumor antigen inhibits the development of cancer in the subject.

In some embodiments of the systems, methods, compositions, uses, and kits its provided herein, the tumor antigens of the tumor avatar are personal tumor antigens. A personal tumor antigen is a tumor antigen that is unique to a single subject and has not been observed in another subject. In some embodiments, the tumor antigens of the tumor avatar are shared tumor antigens. A shared tumor antigen is a tumor antigen that is not unique to a single subject, having been observed in at least one other subject. In some embodiments, the shared tumor antigen is one that is listed in The Cancer Genome Atlas, which is located at cancer.gov. In some embodiments, the shared tumor antigen is one that is listed in the Catalog Of Somatic Mutations In Cancer (COSMIC) database, which is managed by the Wellcome Sanger Institute.

In some embodiments of the systems, methods, compositions, uses, and kits provided herein, the nucleic acid encoding a tumor antigen encodes more than one tumor antigen. In some embodiments, the nucleic acid encoding the tumor antigen is comprised in a nucleic acid library that is administered to the subject. In some embodiments, the nucleic acid library comprises multiple nucleic acids, each encoding a different tumor antigen. In some embodiments, the nucleic acid library comprises multiple nucleic acids encoding the same tumor antigen. The inclusion of multiple copies of a nucleic acid encoding the same nucleic acid allows for expression of the encoded nucleic acid even if one copy is degraded. In some embodiments, the nucleic acid library comprises multiple nucleic acids, cach encoding one or more tumor antigens. In some embodiments, the nucleic acid library comprises some nucleic acids encoding the same tumor antigen and some nucleic acids encoding different tumor antigens. In some embodiments, the nucleic acid or nucleic acid library encodes 2 to 1,000, 2 to 500, 2 to 250, 2 to 100, or 2 to 50 tumor antigens. In some embodiments, the nucleic acid or nucleic acid library encodes 2-100 tumor antigens. In some embodiments, the nucleic acid encodes 2-50 tumor antigens. In some embodiments, the nucleic acid encodes 20-30 tumor antigens. In some embodiments, the nucleic acid encodes about 5, about 10, about 15, about 20, about 25, about 30, about 35, about 40, about 45, or about 50 tumor antigens. In some embodiments, the nucleic acid library comprises nucleic acids that, collectively, encode 2-50 tumor antigens. In some embodiments, the nucleic acid library comprises nucleic acids that, collectively, encode 20-30 tumor antigens. In some embodiments, the nucleic acid library comprises nucleic acids that, collectively, encode about 5, about 10, about 15, about 20, about 25, about 30, about 35, about 40, about 45, or about 50 tumor antigens. In some embodiments, the nucleic acid or nucleic acid library encodes at least 50%, at least 60%, at least 70%, at least 80%, at least 90%, at least 95%, at least 97%, at least 98%, at least 99%, or up to 100% of the tumor antigens present in a tumor cell in the subject. In some embodiments, the nucleic acid or nucleic acid library encodes all or almost all of the tumor antigens present in a tumor cell in the subject. In some embodiments, the nucleic acid or nucleic acid library encodes all or almost all of the genes expressed in a tumor cell in the subject. In some embodiments, the nucleic acid or nucleic acid library encodes all of the tumor antigens present in a tumor in the subject. In some embodiments, the nucleic acid or nucleic acid library encodes a select subset of tumor antigens based on the type of gene from which that tumor antigen arises in the subject. This may include types of genes directly involved in oncogenesis, genes that are the targets of defects in mismatch repair (e.g., genes containing microsatellite regions), or genes involved in early development processes or pluripotent stem cells, which are normally not expressed in healthy adults but are aberrantly expressed in cancer cells. In some embodiments, the nucleic acid library is generated by isolating RNA transcripts from one or more tumor cells in a subject, followed by:
(a) generating a population of puromycin-tagged RNA transcripts, wherein: cach RNA transcript in the population of puromycin-tagged RNA transcripts comprises, in 5′ to 3′ order:
(i) a translation initiation site followed by any multiple of 3 nucleotides not encoding a stop codon;
(ii) an RNA sequence transcribed from a cDNA fragment sequence from a library of cDNA sequences (e.g., from a tumor); and
(iii) a polypeptide-encoding nucleotide sequence which is a multiple of 3 nucleotides in length and encoded by the reading frame initiating at the first 5′ nucleotide of the nucleotide sequence and lacks an in-frame stop codon in that reading frame but contains stop codons in each of the other two reading frames; and wherein the 3′ end of each RNA transcript is joined to the 5′ end of a puromycin-tagged DNA linker;
(b) performing an in vitro translation reaction on the puromycin-tagged RNA transcripts, wherein, for each puromycin-tagged RNA fragment, if the RNA sequence transcribed from a cDNA fragment sequence in a puromycin-tagged RNA transcript is in-frame with the translation initiation site, has no stop codons within that reading frame, and is in frame with the polypeptide-encoding nucleotide sequence, the puromycin will covalently link the translated polypeptide to the puromycin-tagged RNA transcript to form a polypeptide-linked RNA complex; and (c) separating the polypeptide-linked RNA complexes from the RNA transcripts that are not in such complexes, thereby enriching a library of in-frame coding region fragments from a population of RNA transcripts.

In some embodiments of the systems, methods, compositions, uses, and kits provided herein, the tumor antigen is one or more of the proteins provided in International Application No. PCT/US2020/054785, filed Oct. 8, 2019, and published on Apr. 15, 2020 as WO 2021/072075 (“Multi-Domain Protein Vaccine”), which is incorporated by reference herein in its entirety. In some embodiments, the nucleic acid encoding a tumor antigen encodes one of the proteins provided in PCT/US2020/054785. In some embodiments, the tumor antigen-containing cell contains one or more of the proteins provided in PCT/US2020/054785.

In some embodiments of the methods, compositions, and kits provided herein, the tumor antigen is one of the tumor antigens encoded by one or more of the nucleic acids provided in International Application No. PCT/US2021/034131, filed May 26, 2020, and published on Dec. 2, 2021 as WO 2021/242793 (“Nucleic acid artificial mini-proteome libraries”), which is incorporated by reference herein in its entirety. In some embodiments, the nucleic acid encoding a tumor antigen is one of the nucleic acids provided in PCT/US2021/34131. In some embodiments, the tumor antigen-containing cell contains one or more of the tumor antigens encoded by a nucleic acid provided in PCT/US2021/34131. In some embodiments, the nucleic acid library encoding tumor antigens is generated by any one of the methods provided in PCT/US2021/34131.

In some embodiments of the methods provided herein, the method comprises generating tumor antigen-containing cells in vitro or ex vivo by delivering a tumor antigen or one or more nucleic acids encoding a tumor antigen to an isolated population of cells, and administering the tumor antigen-containing cells to the subject at a site that is separate from a tumor in the subject. The isolated population of cells acquire the delivered tumor antigen or express the tumor antigen(s) encoded by the delivered nucleic acids, thereby becoming tumor antigen-containing cells. Once the tumor antigen-containing cells are introduced into the subject, they can be killed, releasing tumor antigens and dead cell components, which trigger an inflammatory response that can facilitate tumor antigen presentation and the generation of an antitumor immune response.

In some embodiments, a tumor antigen-containing cell generated ex vivo and administered to a subject expresses CD1d on its surface. CD1d is a cell surface receptor on which antigens (e.g., lipid antigens) may be presented independently of major histocompatibility complex proteins, for recognition by iNK-T cells. For example, CD1d may present lipid antigens to iNK-T cells expressing a T cell receptor specific to the antigen: CD1d complex. In some embodiments, the CD1d is bound to an antigen capable of being recognized by an invariant natural killer (INK-T) cell. In some embodiments, the antigen is a CD1d-restricted antigen (i.e., an antigen that is presented on CD1d but not class I or class II MHC). Unlike conventional T cells, which express a broad diversity of receptors that recognize antigens presented on MHC proteins, iNK-T cells express a more limited diversity of receptors, which recognize lipid antigens presented on CD1d. The response elicited by binding of iNK-T cells to CD1d-presented antigens has multiple downstream effects, including killing of cells presenting the antigen (e.g., tumor antigen-containing cells) that results in tumor antigen release, after which the antigen may be taken up and presented by other antigen-presenting cells (e.g., dendritic cells). Additionally, INK-T cells activated by antigen recognition may release cytokines (e.g., IFN-γ) that promote dendritic cell maturation in vivo, thereby enhancing the antigen presentation activity of the matured dendritic cells, allowing generation of a CD4+ and/or CD8+ T cell response to the tumor antigen. See, e.g., Fuji et al., 2022. Cancer Sci. 113(3):864-874. Thus, in some embodiments, a tumor antigen-containing cell as described herein comprises on its surface CD1d that is bound to alpha-galactosyl ceramide. In some embodiments, causing the death of a tumor antigen-containing cell in a subject comprises administering an iNK-T cell to the subject, where the iNK-T cell expresses an iNK-T cell receptor that is capable of binding to an antigen presented on CD1d. In some embodiments, the iNK-T cell expresses an iNK-T cell receptor that is capable of binding to alpha-galactosyl ceramide. In some embodiments, the tumor antigen-containing cell comprises CD1d on its surface, where the CD1d is bound to a lipid antigen known in the art to be recognized by iNK-T cells. These and other lipid antigens are described, e.g., in Brennan et al., Nat Rev Immunol. 2013. 13(2):101-117; and O'Keeffe et al., Immunology. 2015. 145(4):468-475. In some embodiments, the CD1d is bound to an antigen selected from α-galactosylceramide (αGalCer), α-galactosyldiacylglycerol (αGalDag), α-glucuronsylceramide (α-GLcACer), α-galacturonosylceramide (α-GalACer), phosphatidyl-myo-inositol mannoside (PIM2), β-glucosylceramide (βGlcCer), plasmalogen lysophosphatidylethanolamine (plasmalogen lysoPE), isoglobotrihexosylceramide (iGb3), and α-glucosyldiacyglycerol (αGlcDAG). In some embodiments, the antigen capable of being recognized by an iNK-T cell is alpha-galactosyl ceramide (αGalCer).

In some embodiments, a tumor antigen-containing cell of a method, system, composition, or kit provided herein is allogeneic with respect to the subject (i.e., the cell was obtained or isolated from a source that is not the subject). Use of an allogeneic cell avoids the need to obtain cells from a subject, which may vary in their capacity to incorporate and present antigens or proliferate ex vivo. For example, introduction of tumor antigens into a cell of a cell line, such as HEK-293 or K562, may be used to establish a line of tumor antigen-containing cells that readily proliferates in culture, producing a substantial volume of tumor antigen-containing cells for administration to the subject. Additionally, as these tumor antigen-containing cells do not necessarily present antigens themselves, but rather release antigen(s) in vivo following cell death to be presented by the subject's own cells, it is not necessary for allogeneic cells to express particular HLA surface antigens.

In some embodiments, a tumor antigen-containing cell of a method, system, composition, or kit provided herein is autologous with respect to a subject (i.e., the cell was obtained or isolated from the subject). Autologous cells express the same HLA surface antigens as other cells (e.g., tumor cells) of the subject, and thus present similar peptide antigens as other cells of the subject. Additionally, use of autologous cells that are less likely to be immediately rejected as foreign following administration, and may thus proliferate after administration, increasing the available supply of tumor antigens in the tumor avatar.

In some embodiments, a tumor antigen-containing cell of a method, system, composition, or kit provided herein is a syngeneic cell (i.e., a cell that is genetically identical to other cells of a subject, or sufficiently genetically identical and immunologically compatible to allow for transplant into the subject). A syngeneic cell may be obtained, e.g., from an identical twin of a subject. Alternatively, a cell may be obtained from a subject and immortalized to produce a cell line of the subject's own cells. Tumor antigens and/or nucleic acids encoding tumor antigens may be introduced into such cells to produce syngeneic tumor antigen-containing cells, which may then be administered to the subject.

A tumor antigen-containing cell may be any cell obtained from a subject (e.g., an autologous cell isolated from an anatomical site that is not a tumor), or any cell obtained from a different source than the subject. In some embodiments, a tumor antigen-containing cell is produced by introducing one or more tumor antigens into a cell of a cell line. In some embodiments, a tumor antigen-containing cell is produced by introducing one or more nucleic acids encoding one or more tumor antigens into a cell of a cell line. Any cell line known in the art may be used to produce a tumor antigen-containing cell from a cell linc. Non-limiting examples of cell lines that may be used in the methods, compositions, systems, or kits provided herein include 786-O, A549, ACHN, BCP-1, BOSC23, BT-20, BT-549, BxPC-3, Caco-2, Caki-1, Calu-3, CCRF-CEM, DAOY, DU145, H295R, H1299, HaCaT, HAP1, HCC-2998, HCT116, HEK293, HEL, HeLa, HepG2, HL-60, Hs 578T, HT-29, HT1080, Huh7, HUVEC, Jurkat, JY, K562, KBM-7, KM12, LAPC4, LNCaP, LOX-IMVI, M14, MCF-7, MCF-7/ADR-RES, MDA-MB-231, MDA-MB-435, MDA-MB-453, MDA-MB-468, MDA-N, MIA PaCa-2, MOLT-4, MRC-5, NCI-60, NCI-H23, NCI-H226, NCI-H460, NCI/ADR-RES, NK-92, NTERA-2, OVCAR-3, OVCAR-8, PANC-1, PC3, Raji, RPMI-7951, Saos-2, SF-268, SF-539, SH-SY5Y, SK-MEL-5, SK-MEL-28, SK-OV-3, SKBR3, SNB-19, SW-620, T-47D, T98G, TF-1, THP-1, U87, U251, U937, VCaP, VG-1, WI-38, and ZR-75-1 cells. In some embodiments, the cell line is selected from the group consisting of K562 cells, HEK293 cells, HeLa cells, A549 cells, Jurkat cells, MDA-MB-231 cells, MCF7 cells, THP-1 cells, Caco-2 cells, HL-60 cells, SH-SY5Y cells, HuH-7 cells, and HT-29 cells.

In some embodiments, a tumor cell lysate is administered to a subject, or used to introduce tumor antigens to a cell to produce a tumor antigen-containing cell that may be administered to the subject. Tumor cell lysates may be prepared by any method known in the art, including repeated freezing and thawing of tumor cells, or contact with a lysis buffer (e.g., alkaline lysis buffer). Lysis of tumor cells releases tumor antigens, allowing the lysate to serve as a source of tumor antigens in the subject, as well as other cellular components (e.g., oxidized phospholipids) that may activate dendritic cells. See, e.g., Hatfield et al., J Immunother. 2008. 31(7):620-632; and Gonzalez et al., Hum Vaccin Immunother. 2014. 10(11):3261-3269. Thus, tumor cell lysates may serve as both tumor antigen source and adjuvant, thereby promoting presntation of tumor antigens to B and T cells in the subject. In some embodiments, the tumor cell lysate is produced by (i) isolating tumor cells from the subject, followed by (ii) lysing the tumor cells, to produce a tumor cell lysate containing antigens present in the subject's tumor.

Aspects of the present disclosure relate to methods of causing the death of tumor antigen-containing cells in a subject at a site that is separate from a tumor in the subject. Once a tumor avatar is generated in the subject by the administration of tumor antigen(s), nucleic acid(s) encoding tumor antigen(s), or tumor antigen-containing cells, the tumor antigen-containing cells of the tumor avatar are induced to die an immunogenic cell death, which releases not only tumor antigens, but also components of the dead cell, such as phospholipids of the cell membrane and oxidation products of the same phospholipids or other inflammatory molecules such as calreticulin (CRT), adenosine triphosphate (ATP), and high-mobility group box 1 (HMGB1). The release of dead cell components triggers an inflammatory response, which leads to the recruitment of innate immune cells, such as monocytes and dendritic cells, which phagocytose components of the nearby environment, such as dead cell components and tumor antigens, and can then present phagocytosed tumor antigens to T and B cells, thereby generating an immune response to the tumor antigens.

Aspects of the present disclosure relate to methods of generating hyperactivated dendritic cells in a subject. Dendritic cells are innate immune cells with multiple functions that link the innate and adaptive arms of the immune system to both initiate a primary adaptive immune response and maintain a memory adaptive response. See, e.g., Wculek et al., Nat Rev Immunol. 2020. 20(1):7-24. Dendritic cells regularly sample proteins from their environment, in a process called “pinocytosis,” and preset peptide fragments of the sampled proteins on cell surface MHC. Dendritic cells routinely sample proteins from one anatomical site, and migrate to the nearest lymph node (draining lymph node), where they will present peptides to T cells that circulate through the lymph node. The first interaction of a CD4+ T cell TCR with a cognate peptide: MHC-II on an APC, such as a dendritic cell in a lymph node, activates the CD4+ T cell. Activated CD4+ T cells, upon subsequent recognition of a target peptide presented on MHC-II by a monocyte, macrophage, or B cell, secrete cytokines that promote activation, survival, and/or proliferation of the antigen-presenting cell. Dendritic cells also cross-present peptide fragments from sampled antigens on MHC-I, through which they can activate CD8+ T cells. Activated CD8+ T cells, upon subsequent recognition of a target peptide presented on MHC-I, secrete cytotoxic granules that induce apoptosis of the antigen-presenting cell. The helper functions of CD4+ T cells and cytotoxic functions of CD8+ T cells are critical for anti-tumor immunity. Thus, activation of CD4+ T cells and CD8+ T cells by dendritic cells plays a key role in initiating an anti-tumor immune response. In addition to initiating a T cell response to antigens, dendritic cells also promote the differentiation of activated T cells into memory T cells. Memory T cells reside in the lymph nodes and peripheral tissues, or circulate in the periphery, and can be rapidly reactivated upon exposure to their target antigen.

Hyperactivated dendritic cells are characterized by the secretion of IL-1β and IL-18. IL-1β is an inflammatory cytokine that is produced in response to potentially harmful stimuli, such as the presence of a pathogen-associated molecular pattern or damage-associated molecular pattern indicating infection or cell death. Unlike many secreted proteins, IL-1β is often produced without a signal sequence and not secreted by conventional secretion pathways. Instead, under inflammatory conditions, large quantities of IL-1β accumulate in the cell and are then released into the extracellular environment following inflammation-induced cell death (pyroptosis). However, dendritic cells may be induced to secrete IL-1β through alternative means that do not require the death of the cell, which allows the dendritic cells to secrete IL-1β for longer periods of time and remain viable so that they can provide other stimuli to T cells. Hyperactivated dendritic cells that secrete IL-1β for prolonged periods of time while remaining viable elicit a more robust CD8+ T cell response to antigens, such as those derived from a tumor lysate, than pyroptotic dendritic cells releasing IL-1β upon cell death. See, e.g., Zhivaki et al. 2020. Cell Reports. 33(7):108381. In some embodiments, hyperactivated dendritic cells are generated by activating caspase-1 in the dendritic cells. In some embodiments, hyperactivated dendritic cells are hyperactivated by activating caspase-4 in the dendritic cells. The activation of caspase-1 triggers canonical inflammasome pathways, while the activation of caspase-4 triggers non-canonical inflammasome pathways, with activation of either pathway resulting in the secretion of IL-1β. See, e.g., Chu et al. Nat Commun. 2018. 996. 9(1):996.

IL-18 is a cytokine that plays multiple roles in stimulating cells of the adaptive immune system. See, e.g., Park et al., Cell Mol Immunol. 2007. 4(5):329-335. First, IL-18 signaling upregulates the cytotoxic functions of natural killer (NK) cells, which increases their ability to kill cancer cells, and also induces the secretion of IFN-γ from NK cells. IFN-γ is a pro-inflammatory cytokine that stimulates the pro-inflammatory activities of T cells and also has direct tumoricidal effects. In addition to its effects on NK cells, IL-18 can enhance type 1 helper T cell (Th1) responses, which are associated with anti-cancer efficacy, in CD4+ T cells. Finally, exposure to IL-18 can upregulate the cytotoxic functions CD8+ T cells (See, e.g., Kohyama et al. Jpn J Cancer Res. 1998. 89:1041-1046).

In some embodiments of the methods provided herein, hyperactivated dendritic cells are generated in vitro or ex vivo by administering a hyperactivating agent to an isolated population of dendritic cells. Any of the hyperactivating agents or nucleic acids encoding hyperactivating agents provided herein may be administered to an isolated population of dendritic cells. The hyperactivation status of the dendritic cells may be confirmed by one of multiple methods known in the art, such as detecting IL-1β and/or IL-18 in cell culture supernatant, detecting IL1B and IL18 RNA in a sample of cells by qPCR, or detecting IL-1β and IL-18 in a sample of cells by intracellular staining and flow cytometry. Hyperactivated dendritic cells may then be administered to the subject at or near the tumor antigen-containing cells in the subject, at a site separate from a tumor. See, e.g., Zhivaki et al. 2020. Cell Reports. 33(7):108381. In some embodiments, the dendritic cells are autologous dendritic cells that are derived from the subject. In some embodiments, the dendritic cells are allogeneic dendritic cells that are not derived from the subject. In some embodiments, dendritic cells are exposed to antigen ex vivo to generate antigen-presenting dendritic cells, which are then administered to the subject. See, e.g., Gu et al., Acata Pharmacol Sin. 2020. 41(7):959-969.

In some embodiments of the methods provided herein, dendritic cells are hyperactivated in vivo by administering a hyperactivating agent, or a nucleic acid encoding a hyperactivating agent, to the subject at a site near the tumor antigen-containing cells of the tumor avatar. Hyperactivated dendritic cells express pro-inflammatory cytokines, such as IL-1β and IL-18, and are more effective at presenting antigens to T cells. IL-1β may itself function as a hyperactivating agent. See, e.g., Liu et al., Cancer Lett. 2021. 520:109-120. The presence of a hyperactivating agent in the body of a subject increases the number of activated immune cells, such as dendritic cells, and promotes their migration to lymph nodes, where antigen presentation activates adaptive immune cells, such as CD4+ T cells, CD8+ T cells, NK-T cells, and B cells. The response of an immune cell to a hyperactivating agent is expected to enhance the generation of an anti-cancer adaptive immune response. When expressed in or administered to an anatomical site separate from a tumor, such as by one of the methods provided herein, a hyperactivating agent can induce the hyperactivation of dendritic cells in the subject, allowing efficient antigen presentation and induction of an anti-tumor response despite the local immunosuppressive environment of the tumor. By providing tumor antigens and causing immunogenic cell death at an anatomical site that is not immunosuppressive, the methods provided herein promote the generation of a robust immune response to a tumor antigen in a subject, allowing for the treatment of cancer in the subject.

A nucleic acid is said to encode a protein, such as a hyperactivating agent, if the nucleic acid contains an open reading frame (RNA) or a sequence that can be transcribed to produce an RNA containing an open reading frame (DNA). An open reading frame (ORF) refers to a nucleic acid sequence that can be translated into a protein by cellular components such as ribosomes and aminoacyl-tRNAs. Generally, an open reading begins with a START codon, such as AUG (RNA) or ATG (DNA), and ends with a STOP codon, such as UAG, UAA, or UGA (RNA) or TAG, TAA, or TGA (RNA), with the number of bases between the last nucleotide of the START codon and the first nucleotide of the STOP codon being a multiple of 3 (e.g., 3, 6, 9, etc.).

In some embodiments of the systems, methods, compositions, uses, and kits provided herein, the hyperactivating agent is a virus or a virus-like particle. In some embodiments, the hyperactivating agent is a vaccinia virus or a particle derived from a vaccinia virus. Vaccinia virus is a DNA virus of the poxvirus family, which is related to the variola virus that causes smallpox. In some embodiments, the hyperactivating agent is a Modified Vaccinia Ankara (MVA) virus or a particle derived from an MVA virus. Modified vaccinia Ankara virus refers to an attenuated vaccinia virus that that was serially passaged multiple times in chicken cells. This serial passage caused the loss of about 10% of the viral genome, and the resulting MVA virus is unable to replicate efficiently in primate cells. As viruses with DNA genomes, vaccinia viruses such as MVA virus are useful as vaccine vectors for delivering exogenous DNA sequences. Additionally, vaccinia viruses such as MVA virus are immunogenic when introduced into a subject. Therefore, the administration of MVA can be used to stimulate cells of the innate immune system and enhance their ability to present peptide antigens encoded by separately administered nucleic acids. See, e.g., Price et al., Vaccine. 2013. 31(39):4231-4234. In some embodiments, the hyperactivating agent is a cowpea mosaic virus (CPMV) or a particle derived from a CPMV. CPMV is an RNA virus of the comovirus genus that infects plants, such as cowpea legumes. Cowpea mosaic virus-like particles have been shown to enhance antitumor immunity in several cancer models, through the induction of IL-12 and IFN-γ expression and enhanced activation of adaptive immune cells (See, e.g., Lizotte et al. Nat Nanotechnol. 2016. 11(3):295-303).

In some embodiments, the virus or virus-like particle is administered to the skin of the subject at or near the tumor antigen-containing cells. In some embodiments, the virus or virus-like particle is administered to the skin of the subject at a site that is separate from a tumor in the subject, and expresses one or more tumor antigens, thereby turning infected cells into tumor avatar cells. In some embodiments, the virus or virus-like particle is administered by skin scarification. Skin scarification is a method of administering a virus, virus-derived particle, or other composition epicutaneously. A sharp object, such as a needle, is used to poke, prod, and scratch the skin multiple times, in order to damage the superficial epidermis. Repeated scratching and abrasion of the skin disrupts the integrity of the superficial epidermis and promotes more efficient infection by virus or virus-derived particles. Administration of a virus, such as MVA virus, by skin scarification causes the activation and recruitment of more T cells than administration by other routes such as intradermal, subcutaneous, or intramuscular administration. Furthermore, skin scarification clicits an immune response characterized by T cells that produce more IFN-γ than T cells elicited by other routes of administration. See, e.g., Pan et al. NPJ Vaccines. 2021. 6(1):1.

In some embodiments of the systems, methods, kits, and uses provided herein, the present disclosure provides an engineered receptor that is capable of binding to a tumor antigen, the engineered receptor comprising (a) an extracellular domain that is capable of binding to a tumor antigen; (b) a transmembrane domain; and (c) an intracellular domain of CLEC9A. In some embodiments, the tumor antigen comprises a ligand that is capable of being bound by the engineered receptor. Non-limiting examples of ligands include WH and CBP peptides (bound by CLEC9A); CD40L (bound by CD40); anti-CD40 antibody or scFv (bound by CD40); anti-CD19 antibody or scFv (bound by CD19); anti-Stefin A antibody (bound by Stefin A). See, e.g., Gou et al., Theranostics. 2021. 11(15):7308-7321. Expression of the nucleic acid by cells, or internalization of an administered engineered receptor, causes the engineered receptor to be expressed on the cell surface, with the infected cell then accumulating tumor antigens on its surface. The accumulation of tumor antigens on the cell surface thus turns the cell into a tumor antigen-containing cell, which can be killed or phagocytosed by immune cells. In some embodiments, the extracellular domain is connected to the transmembrane domain by one or more linker domains. In some embodiments, the intracellular domain of CLEC9A is connected to the transmembrane domain by one or more linker domains. In some embodiments, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the glycine spacer comprises at least 3 glycines. In some embodiments, the glycine spacer comprises a sequence set forth in SEQ ID NO: 1 (GGGS), SEQ ID NO: 2 (GGGSGGG) or SEQ ID NO: 3 (GGG). In some embodiments, the transmembrane domain is located N-terminal to the signaling domain, the hinge domain is located N-terminal to the transmembrane domain, the linker is located N-terminal to the hinge domain, and the extracellular binding domain is located N-terminal to the linker. In some embodiments, the engineered receptor further comprises a hinge domain connecting the extracellular domain to the transmembrane domain, the extracellular domain to one or more extracellular linker domains, and/or one or more extracellular linker domains to the transmembrane domain. In some embodiments, the intracellular domain of CLEC9 comprises an amino acid sequence with at least 90%, at least 95%, at least 96%, at least 97%, at least 98%, at least 99%, or up to 100% sequence identity to the amino acid sequence of SEQ ID NO: 4. In some embodiments, the intracellular domain of CLEC9A comprises the amino acid sequence of SEQ ID NO: 4. CLEC9A is expressed on some dendritic cells, and facilitates the internalization and processing of extracellular antigens, so that they can be cross-presented on MHC-I to stimulate CD8+ T cells. See, e.g., Sancho et al. Nature. 2009. 458(7240):899-903.

In some embodiments, the hyperactivating agent administered to the subject at a site near the tumor antigen-containing cells of the tumor avatar is an oxidized phospholipid. An oxidized phospholipid is a phospholipid that has been modified by the loss of one or more electrons, which may result in the loss of some atoms of the phospholipid and/or the formation of a covalent bond with another molecule, thereby producing a phospholipid with a different structure. In some embodiments, the oxidized phospholipid is an oxidation product of 1-palmitoyl-2-arachidonyl-sn-glycero-3-phosphorylcholine (PAPC). PAPC is a naturally occurring lipid that is commonly found in mammalian cell membranes. Dead cells with disrupted membranes may release membrane phospholipids, in both intact forms, which may be oxidized after release, or oxidized forms. An oxidation product of PAPC is referred to herein as OxPAPC. In some embodiments, the the oxidized phospholipid is 1-palmitoyl-2-glutaryl-sn-gylcero-3-phosphocholine (PGPC). PGPC is one example of a lipid that is produced by oxidation of PAPC. Such oxidation products are commonly released from dying cells, and their presence thus serves as a signal of cell death to nearby cells. See, e.g., Zanoni et al., Immunity. 2017. 47(4):697-709.e3. Oxidized phospholipids, such as oxidation products of PAPC, have been shown to activate the inflammasomes of innate immune cells such as dendritic cells, promoting IL-1β secretion and CCR7-dependent hypermigration to lymph nodes by dendritic cells, which results in generation of a robust IFN-γ+CD8+ T cell response and anti-tumor immunity. See, e.g., Zhivaki et al. 2020. Cell Reports. 33(7):108381. OxPAPC is capable of inducing secretion of IL-1β through the non-canonical inflammasome pathway by activating caspase-4 in humans and caspase-11 in mice, without inducing inflammsome-mediated cell death (pyroptosis) that is typically required for IL-1β release. See, e.g., Chu et al. Nat Commun. 2018. 996. 9(1):996.

In some embodiments of the methods provided herein, the method comprises generating an oxidized phospholipid, such as an oxidation product of PAPC, in the subject. An oxidized phospholipid may be generated by any of multiple methods known in the art, including exposing the subject to a source of energy, a sensitizing reagent, and/or an oxidizing reagent. In some embodiments, the method comprises administering one or more phospholipids to the subject, and causing the oxidation of the administered phospholipid(s) in the subject. In some embodiments, the method comprises oxidizing one or more phospholipids already present in the subject. In some embodiments, the generating of an oxidized phospholipid comprises causing cell death in the subject.

In some embodiments of the methods provided herein, the step of causing cell death and/or generating an oxidized phospholipid comprises administering an oxidizing reagent to the subject. An oxidizing reagent is a composition, such as a molecule, that is capable of oxidizing another molecule. An oxidizing reagent may comprise one or more free radicals. Contact between an oxidizing reagent and a phospholipid, results in the oxidizing reagent donating an electron to the phospholipid, thereby oxidizing the other molecule. The oxidized phospholipid may then react with third molecule, forming a bond with the third molecule to produce an oxidation product comprising the phospholipid and the third molecule. In some embodiments, a cytotoxic agent is administered to kill cells in or near the tumor avatar. Oxidized phospholipids arc readily generated in dead cells, which may be released as the dead cell decays. In some embodiments, the cytotoxic agent is a chemotherapeutic agent. In some embodiments, the cytotoxic agent is squaric acid, cpirubicin, bleomycin, bortezomib, cyclophosphamide, doxorubicin, idarubicin, mitoxantrone, or oxaliplatin. In some embodiments, the cytotoxic agent is squaric acid. Squaric acid, also known as quadratic acid or 3,4-dihydroxycyclobut-3-ene-1,2-dione, is an immunogenic organic acid that is known to cause cell death, activate dendritic cells, and cross-link peptide epitopes, which may be presented in tandem in draining lymph nodes. See, e.g., Mookerjee et al., Bioimpacts. 2018. 8(3):211-221. Non-limiting examples of other cytotoxic agents are provided in Pol et al., Oncoimmunology. 2015. 4(4):31008866, which is incorporated by reference herein in its entirety.

In some embodiments of the methods provided herein, the step of causing cell death comprises administering a nucleic acid encoding an antigen to the subject at or near the site of the tumor avatar, and administering to the subject an antibody specific to the antigen. Binding of the antibody to the antigen can facilitate recruitment and activation of natural killer (NK) cells, which kill antibody-bound cells via antibody-dependent cell-mediated cytotoxicity (ADCC). Killing of cells of the tumor avatar thus results in immunogenic cell death, promoting an inflammatory response, and release of tumor antigens, allowing for antigen uptake and presentation. In some embodiments, binding of the antibody to the antigen forms an immune complex that is capable of being bound by dendritic cells and/or neutrophils. Binding of Fc receptors on dendritic cells and/or neutrophils to Fc domains of antibodies in the immune complex stimulates uptake of the immune complex by dendritic cells and/or neutrophils. Uptake of the immune complex activates dendritic cells and neutrophils, promoting antigen presentation and activation of CD4+ T cell and CD8+ T cell responses towards antigens of the immune complex. See, e.g., Regnault et al., J Exp Med. 1999. 189(2):371-380; and Mysore et al., Nat Commun. 2021. 12(1):4791.

In some embodiments, the surface antigen and one or more tumor antigens are encoded by the same nucleic acid. In some embodiments, the nucleic acid encodes CD20, and the method comprises administering to the subject an anti-CD20 antibody. Non-limiting examples of anti-CD20 antibodies include rituximab and obinituzumab. In some embodiments, the nucleic acid encodes epidermal growth factor receptor (EGFR) or a portion of EGFR that lacks a signal transduction domain, and the method comprises administering to the subject an anti-EGFR antibody. In some embodiments, the anti-EGFR antibody is cetuximab. In some embodiments, the nucleic acid encodes an antigen selected from the group consisting of CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49c, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD14, CDw145, CD146, CD147, CD148, CD150, CD152, CD152, CD153, CD154, CD155, CD156a, CD156b, CD156c, CD157, CD158b1, CD158b2, CD158d, CD158c1/c2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD272, CD272, CD273, CD274, CD275, CD276, CD277, CD278, CD279, CD280, CD281, CD282, CD283, CD284, CD286, CD288, CD289, CD290, CD292, CD293, CD294, CD295, CD296, CD297, CD298, CD299, CD300a, CD300c, CD300c, CD301, CD302, CD303, CD304, CD305, CD306, CD307a, CD307b, CD307c, CD307d, CD307c, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362 and CD363.

In some embodiments of the methods provided herein, the step of causing cell death and/or generating hyperactivated dendritic cells comprises administering a sensitizing agent, or a nucleic acid encoding a sensitizing agent, to a subject, and exposing the sensitizing agent to an energy source. In some embodiments, the energy source causes a reaction in the sensitizing agent, such as the generation of reactive oxygen species, resulting in the oxidation of phospholipids in nearby cells and/or the death of nearby cells. In some embodiments the sensitizing agent acts as a catalyst or cofactor, allowing the energy source to more readily generate reactive oxygen species in the proximity of the sensitizing agent.

In some embodiments of the systems, methods, compositions, uses, and kits provided herein, the sensitizing agent is a photosensitizing agent, and the energy source is light. In some embodiments, the photosensitizing agent is a mini singlet oxygen generator (miniSOG). See, e.g., Qi et al. Proc Natl Acad Sci U S A. 2012. 109(19):7499-7504.

In some embodiments, the light is ultraviolet light, visible light, or infrared light. In some embodiments, the light is ultraviolet light. Ultraviolet light is light with wavelengths between 100-400 nm, inclusive. Ultraviolet light includes three categories: UV-A (315-400 nm), UV-B (280-315 nm), and UV-C (100-315 nm). In some embodiments, the light is visible light. Visible light is light that is perceptible to the human eye, which includes light with wavelengths between 380-740 nm, inclusive. Infrared light is light with wavelengths between 780 nm and 1 mm. Infrared light includes three categories: IR-A (780-1.4 μm), IR-B (1.4-3 μm), and IR-C (3 μm-1 mm). Non-limiting examples of photosensitizing agents include, but are not limited to, riboflavin, verteporfin, methoxsalen, porfimer sodium, carprofen, aminolevulinic acid, tiaprofenic acid, benzophenone, protoporphyrin, trioxsalen, acetophenone, motexafin lutetium, motexafin gadolinium, hexaminolevulinate, rostaporfin, cyamemazine, titanium dioxide, temoporfin, talaporfin, bergapten, methyl aminolevulinate, dihematoporphyrin ether, cfaproxiral, padeliporfin, indapamide, leuprolide, lovastatin, polythiazide, hydroflumethiazide, pitolisant, lamotrigine, chloroquine, trimethoprim, sulfamethoxazole, minocycline, levofloxacin, diclofenac, promethazine, amiodarone, furosemide, ketoconazole, dronedarone, prochlorperazine, enalapril, bupropion, diltiazem, triamterene, simvastatin, methotrexate, misoprostol, hydrochlorothiazide, nadolol, gemfibrozil, hydroxychloroquine, lisinopril, losartan, oxcarbazepine, gabapentin, cyclobenzaprine, escitalopram, enalaprilat, stiripentol, simeprevir, cobimetinib, enoximonc, febuxostat, nilotinib, thiothixene, pipotiazine, methotrimeprazine, isocarboxazid, dasatinib, estazolam, captopril, zopiclone, itraconazole, sertraline, flucytosine, diphenhydramine, thalidomide, ketoprofen, cromoglicic acid, doxorubicin, oxaprozin, lomefloxacin, tipranavir, quinidine, chlorothiazide, flupentixol, tacrolimus, diflunisal, perphenazine, trifluoperazinc, acetazolamide, mefenamic acid, tetracycline, ctodolac, carbinoxamine, hexachlorophene, esomeprazole, paroxetine, flurbiprofen, methazolamide, moexipril, pontosan polysulfate, thioridazine, fluphenazine, demeclocycline, ethionamide, sulindac, piroxicam, benazepril, fluorouracil, haloperidol, fosinopril, nabilone, chlorpromazine, nabumetone, ketorolac, acitretin, cyproheptadine, loxapine, eszopiclone, nisoldipine, clozapine, pyrazinamide, chlorthalidone, valproic acid, metoprolol, sulfisoxazole, doxycycline, methyclothiazide, azithromycin, ramipril, pravastatin, interferon alfa-2b, clofazimine, methylene blue, trovafloxacin, dapsone, naproxen, nalidixic acid, vemurafenib, voriconazole, ciprofloxacin, celecoxib, bumetanide, isotretinoin, glyburide, etretinate, dabrafenib, imatinib, vandetanib, hemoporfin, and fosdenopterin. In some embodiments, the photosensitizing agent is encoded by a nucleic acid that is administered to the subject. In some embodiments, a nucleic acid encoding a protein that produces the photosensitizing agent is administered to the subject.

In some embodiments, the sensitizing agent is an acoustic sensitizer, and the energy source is ultrasound. An acoustic sensitizer facilitates the transfer of energy from sound waves into the tissue containing the acoustic sensitizer. Ultrasound refers to sound waves with frequencies above the upper limit of human hearing. The upper limit of human hearing varies depending on age and the volume of a particular sound, but is generally about 20,000 Hz. Thus, ultrasound waves generally have frequencies above 20,000 Hz. In some embodiments, the acoustic sensitizer is an acoustic coupling medium, which facilitates the transfer of energy from a transducer, the source of ultrasound waves, into the tissue, where the ultrasound waves may cause cell death and/or the generation of oxidized phospholipids to promote dendritic cell hyperactivation. Non-limiting examples of acoustic sensitizing agents include ultrasound gel, mineral oil, white petrolatum, and water. See, e.g., Casarotto et al., Arch Phys Med Rehabil. 85(1):162-165. Any energy source that is capable of penetrating tissue and inducing cell death at the site of the tumor avatar may be used.

In some embodiments of the systems, methods, compositions, uses, and kits provided herein, the hyperactivating agent, is a LysM-containing protein. In some embodiments, the LysM-containing protein is a Listeria monocytogenes p60 protein or a fragment thereof. The Listeria monocytogenes p60 protein, particularly the N-terminal domain, enhances the ability of dendritic cells to activate NK cells and stimulate IFN-γ secretion, which play important roles in anti-cancer immunity (See, e.g., Schmidt et al., PLoS Pathog. 2011. 7(11):e1002368). In some embodiments, the LysM-containing protein comprises a LysM1 amino acid sequence. In some embodiments, the LysM-containing protein comprises a LysM1 amino acid sequence and one or more SH3 sequences. In some embodiments, the LysM-containing protein further comprises one or more tumor antigens. In some embodiments, the LysM-containing protein comprises a LysM1 peptide with the amino acid sequence of SEQ ID NO: 5. An example of a DNA sequence encoding L. monocytogenes p60 is given by Accession No. AF532267, and reproduced as SEQ ID NO: 6. An example of an amino acid sequence of L. monocytogenes p60 is given by Accession No. Q83TQ3, and reproduced as SEQ ID NO: 7. In some embodiments the LysM-containing protein comprises an amino acid sequence with at least 90%, at least 91%, at least 92%, at least 93%, at least 94%, at least 95%, at least 96%, at least 97%, at least 98%, at least 99%, or up to 100% sequence identity to the amino acid sequence of SEQ ID NO: 5. In some embodiments, the LysM-containing protein comprises an amino acid sequence with at least 95% sequence identity to the amino acid sequence of SEQ ID NO: 5. In some embodiments, the LysM-containing protein comprises the amino acid sequence of SEQ ID NO: 5. In some embodiments, the LysM1 protein is linked to a tumor antigen. In some embodiments, the LysM1 protein is the N-terminus of a larger protein. In some embodiments, the LysM1 protein alone or the LysM1 protein linked to additional protein domains is secreted.

In some embodiments of the systems, methods, compositions, uses, and kits provided herein, the hyperactivating agent comprises a tRNA synthetase domain. In some embodiments, the tRNA synthetase is a tryptophanyl-tRNA synthetase (WARS) or a cysteinyl-tRNA synthetase (CARS). In some embodiments, the tRNA synthetase comprises an amino acid sequence with at least 90%, at least 95%, at least 96%, at least 97%, at least 98%, at least 99%, or up to 100% sequence identity to the amino acid sequence of SEQ ID NO: 8 or SEQ ID NO: 9. In some embodiments, the tRNA synthetase comprises the amino acid sequence of SEQ ID NO: 8. In some embodiments, the tRNA synthetase comprises the amino acid sequence of SEQ ID NO: 9. In some embodiments, the hyperactivating agent is a fusion protein comprising (a) a tRNA synthetase domain comprising an amino acid sequence with at least 90%, at least 95%, at least 96%, at least 97%, at least 98%, at least 99%, or up to 100% sequence identity to the amino acid sequence of SEQ ID NO: 8 or SEQ ID NO: 9; and (b) a grp170 domain comprising an amino acid sequence with at least 90%, at least 95%, at least 96%, at least 97%, at least 98%, at least 99%, or up to 100% sequence identity to the amino acid sequence of SEQ ID NO: 16.

In some embodiments of the systems, methods, compositions, uses, and kits provided herein, the hyperactivating agent is an HMGB1. In some embodiments, the HMGB1 comprises an amino acid sequence with at least 90%, at least 95%, at least 96%, at least 97%, at least 98%, at least 99%, or up to 100% sequence identity to the amino acid sequence of SEQ ID NO: 11. In some embodiments, the HMGB1 comprises the amino acid sequence of SEQ ID NO: 11.

In some embodiments of the systems, methods, compositions, uses, and kits provided herein, the hyperactivating agent is a granzyme. In some embodiments, the granzyme comprises an amino acid sequence with at least 90%, at least 95%, at least 96%, at least 97%, at least 98%, at least 99%, or up to 100% sequence identity to the amino acid sequence of SEQ ID NO: 12. In some embodiments, the granzyme comprises the amino acid sequence of SEQ ID NO: 12.

In some embodiments of the systems, methods, compositions, uses, and kits provided herein, the hyperactivating agent is a fusion protein comprising a Toll-like receptor-stimulating domain and a glucose-regulated protein (grp170) domain. A Toll-like receptor-stimulating domain refers to a molecule, such as a protein, lipid, carbohydrate, or nucleic acid, that is capable of interacting with a Toll-like receptor (TLR) to transduce an activating signal through the TLR. Non-limiting examples of TLR agonists include peptidoglycan, double-stranded RNA, CpG DNA, lipopolysaccharide, heat shock protein, fibrinogen, profilin, and flagellin. In some embodiments, the Toll-like receptor-stimulating domain is selected from the group consisting of a heat shock protein, a fibrinogen domain, a profilin domain, and a flagellin domain.

In some embodiments of the systems, methods, compositions, uses, and kits provided herein, the hyperactivating agent is a flagellin-grp170 fusion protein. Flagellin-grp170 is a protein comprising an amino acid sequence derived from bacterial flagellin; and an amino acid sequence derived from glucose-regulated protein (grp170). Flagellin is a protein component of bacterial flagella, and many vertebrates, including humans, express receptors that upregulate multiple immune responses, including the activation of dendritic cells, following detection of flagellin. A flagellin-grp170 protein induces multiple responses that enhance antitumor immunity, including elevated expression of IFN-γ and IL-12, and increased infiltration of CD8+ T cells into the tumor microenvironment (See, e.g., Yu et al. Cancer Res. 2013. 73(7):2093-2103). An example of a DNA sequence encoding Salmonella enterica flagellin is given by Accession No. AAL20871, and reproduced as SEQ ID NO: 13. An example of an amino acid sequence of Salmonella enterica flagellin is given by Accession No. P06179, and reproduced as SEQ ID NO: 14. An example of a DNA sequence encoding grp170 is given by Accession No. AF228709, and reproduced as SEQ ID NO: 15. An example of an amino acid sequence of grp170 is given by Accession No. Q9Y4L1, and reproduced as SEQ ID NO: 16. In some embodiments, the flagellin-grp 170 comprises an N-terminal domain of a flagellin, a C-terminal domain of a flagellin, and a C-terminal domain of a grp170 protein, with each domain being connected by a flexible linker. In some embodiments the flagellin-grp170 comprises amino acid sequences with at least 90%, at least 91%, at least 92%, at least 93%, at least 94%, at least 95%, at least 96%, at least 97%, at least 98%, at least 99%, or up to 100% sequence identity to each of amino acid sequences of SEQ ID NOs: 10, 17, and 18. In some embodiments, the flagellin-grp170 protein comprises amino acid sequences with at least 95% to each of the amino acid sequences of SEQ ID NOs: 10, 17, and 18. In some embodiments, the flagellin-grp 170 comprises the amino acid sequences of SEQ ID NOs: 10, 17, and 18.

In some embodiments of the systems, methods, compositions, uses, and kits provided herein, the hyperactivating agent is a cowpea mosaic virus (CPMV) coat protein, CPMV virus particle, or CPMV virus-like particle. The CPMV capsid comprises a structure formed by multiple copies of large (CPMV-L) and small (CPMV-S) capsid proteins. Viral and virus-like particles containing these cowpea mosaic virus proteins have been shown to enhance antitumor immunity in several cancer models, through the induction of IL-12 and IFN-γ expression and enhanced activation of adaptive immune cells (See, e.g., Lizotte et al. Nat Nanotechnol. 2016. 11(3):295-303). Both capsid proteins are formed by translation of a single polypeptide, referred to as M, VP60, or RNA2 polypeptide, followed by cleavage of the polypeptide to release individual proteins, which include CMPV-L, CPMV-S, and other proteins. In some embodiments, the CPMV coat protein is a large (CPMV-L) coat protein. In some embodiments, the CPMV coat protein is a small (CPMV-S) coat protein. An example of a DNA sequence encoding an RNA2 polypeptide is given by Accession No. X00729, and reproduced as SEQ ID NO: 19. An example of an amino acid sequence of CPMV RNA2polypeptide is given by Accession No. P03599, and reproduced as SEQ ID NO: 20. An example of an amino acid sequence of CPMV-S is given by amino acids 834-1022 of Accession No. P03599, and reproduced as SEQ ID NO: 21. An example of an amino acid sequence of CPMV-L is given by amino acids 460-833 of Accession No. P03599, and reproduced as SEQ ID NO: 22. In some embodiments the CPMV-S protein comprises an amino acid sequence with at least 90%, at least 91%, at least 92%, at least 93%, at least 94%, at least 95%, at least 96%, at least 97%, at least 98%, at least 99%, or up to 100% sequence identity to the amino acid sequence of SEQ ID NO: 21. In some embodiments, the CPMV-S protein comprises an amino acid sequence with at least 95% sequence identity to the amino acid sequence of SEQ ID NO: 21. In some embodiments, the CPMV-S protein comprises the amino acid sequence of SEQ ID NO: 21. In some embodiments the CPMV-L protein comprises an amino acid sequence with at least 90%, at least 91%, at least 92%, at least 93%, at least 94%, at least 95%, at least 96%, at least 97%, at least 98%, at least 99%, or up to 100% sequence identity to the amino acid sequence of SEQ ID NO: 22. In some embodiments, the CPMV-L protein comprises an amino acid sequence with at least 95% sequence identity to the amino acid sequence of SEQ ID NO: 22. In some embodiments, the CPMV-L protein comprises the amino acid sequence of SEQ ID NO: 22.

In some aspects, the present disclosure provides a fusion protein comprising an anchoring domain and a cytokine domain. In some embodiments of the systems, methods, compositions, uses, and kits provided herein, the hyperactivating agent is a fusion protein comprising a cytokine domain and an anchoring domain. An anchoring domain is a protein domain that is capable of binding to another molecule, such as a lipid or protein that is commonly found on a cell surface or in an extracellular space, such that binding of the anchoring domain to the other molecule prevents the protein containing the anchoring domain from diffusing freely. A protein comprising an anchoring domain is more likely to remain in a local anatomical site, such as the tumor avatar where it was produced, and exert a biological effect in that site rather than diffusing or circulating elsewhere in the body. In some embodiments, the cytokine domain comprises a cytokine or a fragment thereof, wherein the cytokine is selected from the group consisting of GM-CSF, MIP-1α, IL-1β, IL-2, IL-6, IL-8, IL-12, IL-17, IL-18, IP-10, IFN-γ, and TNF-α. In some embodiments, the cytokine domain comprises a cytokine or a fragment thereof, wherein the cytokine is selected from the group consisting of GM-CSF, MIP-1α, IL-1β, IL-6, IL-8, IL-17, IL-18, IP-10, IFN-γ, and TNF-α. In some embodiments, the cytokine domain comprises IL-1β or a fragment thereof. See, e.g., Han et al. Sci Immunol. 2021. 6(59):eabc6998.

In some embodiments, the anchoring domain is an antibody or an antigen-binding fragment thereof. In some embodiments, the antibody or antigen-binding fragment thereof binds to an antigen that is present in the site at which the tumor antigen, nucleic acid encoding the tumor antigen, or tumor antigen-containing cells are administered. In some embodiments, the antibody or antigen-binding fragment thereof binds to an antigen selected from the group consisting of fibronectin extra domain A, fibronectin extra domain B, and clastin. In some embodiments, the antibody or antigen-binding fragment thereof binds to a cell adhesion molecule. Cell adhesion molecules are known in the art, and mediate interactions, such as binding, between cells. Non-limiting examples of cell adhesion molecules include integrins, cadherins, and selectins. See, e.g., Harjunpää et al. Front Immunol. 2019. 10:1078.

In some embodiments, the anchoring domain is a lumican domain. Lumican is a protein that binds to collagen, which is abundant in bones, muscles, and skin, with extracellular matrices containing large amounts of collagen. A fusion protein containing a lumican domain thus remains in the extracellular space surrounding the cell from which it is secreted. See, e.g., Momin et al. Sci Transl Med. 2019. 11(498):eaaw2614. An IL-1β-lumican fusion protein produced by cells of a tumor avatar will thus remain in the tumor avatar, with the IL-1β domain exerting a pro-inflammatory effect on immune cells of the tumor avatar, such as dendritic cells. An example of a DNA sequence encoding the amino acid sequence of a precursor form of human IL-1β is given by Accession No. X56087.1, and reproduced as SEQ ID NO: 23. An example of an amino acid sequence of a mature form of IL-1β is given by Accession No. C9JVK0, and reproduced as SEQ ID NO: 24. An example of a DNA sequence encoding a lumican domain is given by Accession No. U18728.1, and reproduced as SEQ ID NO: 25. An example of an amino acid sequence of a lumican domain is given by Accession No. P51884, and reproduced as SEQ ID NO: 31. In some embodiments, the fusion protein comprises an IL-1β domain and a lumican domain, with each domain being connected by a flexible linker. In some embodiments the IL-1β domain comprises an amino acid sequence with at least 90%, at least 91%, at least 92%, at least 93%, at least 94%, at least 95%, at least 96%, at least 97%, at least 98%, at least 99%, or up to 100% sequence identity the amino acid sequences of SEQ ID NO: 24. In some embodiments the anchoring domain comprises an amino acid sequence with at least 90%, at least 91%, at least 92%, at least 93%, at least 94%, at least 95%, at least 96%, at least 97%, at least 98%, at least 99%, or up to 100% sequence identity the amino acid sequences of SEQ ID NO: 26. In some embodiments, the IL-1β domain comprises the amino acid sequence of SEQ ID NO: 24. In some embodiments, the anchoring domain comprises the amino acid sequence of SEQ ID NO: 26.

In some embodiments, the anchoring domain is an A3 domain of von Willebrand factor. An example of a DNA sequence encoding von Willebrand factor domain is given by Accession No. X04385.1, and reproduced as SEQ ID NO: 27. An example of an amino acid sequence of a von Willebrand factor is given by Accession No. P04275, and reproduced as SEQ ID NO: 28. In some embodiments, the A3 domain of von Willebrand factor comprises amino acids 910-1113 of SEQ ID NO: 28. See, e.g., Lankhof et al. Thromb Haemost. 1996. 75(6):950-958.

In some embodiments of the systems, methods, compositions, uses, and kits provided herein, the hyperactivating agent further comprises a tumor antigen. Physically linking a tumor antigen to a hyperactivating agent promotes presentation of the tumor antigen by hyperactivated dendritic cells, which promotes the effective generation of T cells and antibodies specific to the tumor antigen.

### Generating Adaptive Immune Response

Aspects of the present disclosure relate to systems, methods, compositions, uses, and kits for generating an immune response to tumor antigens in a subject. An immune response to an antigen refers to an action of the immune system that specifically targets the antigen. For example, CD8+ T cells containing T cell receptors that recognize the antigen may kill cells presenting the antigen on their surface, and CD4+ T cells may send activating signals to other cells, such as DCs, B cells, and macrophages, that present the antigen. Additionally, B cells may produce antibodies that specifically bind to an antigen, neutralizing the antigen, and/or marking it for phagocytosis by other cells.

In some embodiments of the methods provided herein, the method induces the production of one or more inflammatory cytokines in the subject. Inflammation is a biological response to the presence of a harmful stimulus, such as a pathogen, dead or damaged cells, or an irritant. Inflammation involves a set of responses to facilitate clearance of the stimulus, including dilation of blood vessels to promote blood flow to the affected area, and increased permeability of blood vessels to allow circulating cells to move from blood vessels into the affected tissue. Inflammation also involves the production of cytokines and chemokines, to direct responses by nearby immune cells, and to recruit immune cells to the affected tissue, respectively. The production of inflammatory cytokines, in particular, facilitates recruitment of innate immune cells, such as monocytes, macrophages, and dendritic cells, to the affected tissue. Once recruited, monocytes, macrophages, and dendritic cells sample proteins and other molecules from the site of inflammation, then migrate to a nearby lymph node (draining lymph node), where they present peptide fragments from sampled proteins to T cells. Sampled proteins may be degraded into peptide fragments in the phagosome and loaded directly onto MHC-II, which is then exported to the cell surface for presentation to CD4+ T cells. Presentation of sampled proteins to CD8+ T cells may occur in a similar manner, in which peptide fragments are loaded onto MHC-I embedded in the phagosome membrane, which is then exported to the cell surface, or through the cytosolic pathway of cross-presentation. In this pathway, the sampled protein is degraded by the proteasome, with peptide fragments being imported into the endoplasmic reticulum, where they are loaded onto MHC-I, which is then exported to the cell surface. See, e.g., Joffre et al., Nat Rev Immunol. 2012. 12(8):557-569. In some embodiments, the method induces the production of one or more inflammatory cytokines selected from the group consisting of GM-CSF. MIP-1α, IL-1β, IL-6, IL-8, IL-12, IL-17, IL-18, IP-10 IFN-γ, and TNF-α. In some embodiments, the method induces the production of IL-1β and IL-18. In some embodiments, the method induces the production of IFN-γ. In some embodiments, the method induces the production of TNF-α.

In some embodiments of the methods provided herein, antibodies specific to the tumor antigen are generated in the subject. In some embodiments, an antibody selected from the group consisting of IgM, IgA, IgG, and IgE is generated in the subject.

The generation of antibodies to an antigen, such as a tumor antigen, occurs in a subject after an epitope, or molecular structure, on the antigen interacts with a surface-bound antibody on a B cell surface. After development but prior to activation, B cells express a surface-bound form of an antibody. The surface-bound antibody, referred to as the B cell receptor, is anchored to the B cell membrane via an antibody constant region. Binding of the antigen to the surface-bound antibody triggers signal transduction inside the B cell that results in B cell activation. Activated B cells produce soluble forms of antibodies and secrete them into circulation. Binding of secreted antibodies to target antigens can neutralize biological functions of the bound antigen, such as toxicity of a toxin, or prevent a bound receptor can being stimulated by a cognate ligand. Binding of antibodies to cells expressing the antigen, such as cancer cells or tumor cells expressing a tumor antigen, can promote effector functions of other cells of the immune system, such as neutrophils and natural killer (NK) cells, which interact with antibody constant regions. For example, neutrophils may engulf (phagocytose) antibody-bound cells, and both neutrophils and NK cells may release cytotoxic granules that kill antibody-bound cells. Antibodies specific to tumor antigens may therefore alleviate cancer symptoms in a subject by directly interfering with cancer cell and tumor growth, as well as promoting an anti-tumor immune response. Methods of determining whether antibodies to a given antigen are present in a subject are well known in the art, and include ELISA, neutralization assay, western blot, fluorescence microscopy, and immunohistochemistry.

In some embodiments of the methods provided herein. T cells specific to the tumor antigen are generated in the subject. T cells are cells of the adaptive immune system that recognize antigens via interaction between a T cell receptor on the T cell surface and an antigen presentation complex on the surface of another cell. The T cell receptor (TCR) is a molecule (e.g., protein) found on the surface of T cells (i.e., a type of lymphocytes, formed in the thymus which expresses the TCR) which is responsible for recognizing fragments of antigen as peptides bound to major histocompatibility complex (MHC) molecules. The TCR comprises an αβ (alpha-beta) antigen sensing subunit (distinct to each T cell and having an alpha chain and a beta chain), which is non-covalently linked to the signaling subunit, collectively known as CD3 signaling complex (the CD3εγ (CD3epsilon-CD3gamma) heterodimer, CD3εδ (CD3epsilon-CD3delta) heterodimer, and the CD3ζζ (CD3zeta-CD3zeta) homodimer, cach of which may be referred to herein as a CD3 signaling subunit of the CD3 signaling complex).

When the TCR engages with the MHC, through presentation of an antigen to the TCR, the TCR recognizes the antigen and initiates signal transduction. This signal transduction occurs through the various components of the signaling domains (e.g., CD3 signaling subunits) associated with the TCR, which form the TCR complex. This signaling can occur through a variety of mechanisms known in the art, for example by a series of biochemical events mediated by associated enzymes, co-receptors, specialized adaptor molecules, and activated or released transcription factors.

The effects of signal transduction through the TCR following antigen recognition depend on multiple factors, such as the type of T cell, the presence of cytokines signaling through other receptors on the T cell surface, and co-stimulatory signals from other receptors on the surface of the antigen-presenting cell. Generally, CD8+ T cells recognize peptides presented on class I MHC (MHC-I) molecules, while CD4+ T cells recognize peptides presented on class II MHC molecules. Most cells, except for red blood cells, express MHC-I proteins, and degrade intracellular proteins to produce peptides that are then presented in MHC-I proteins on the cell surface. CD8+ T cells that strongly recognize self-antigens, or peptides encoded by the unmutated genome, are prevented from maturing, so mature T cells only weakly recognize self-antigens weakly or recognize non-self-antigens, such as peptides encoded by pathogens or cells in which a gene is mutated. Strong binding of a CD8+ T cell TCR to peptide: MHC on the surface of another cell thus indicates that the cell is either infected with a pathogen or expressing a non-self-antigen. CD8+ T cells release cytotoxic granules to induce apoptosis in such cells and thereby inhibit pathogen replication or proliferation of the cancerous cell. In some embodiments of the methods provided herein, the T cells generated in the subject are CD8+ T cells.

In some embodiments of the methods provided herein, the T cells generated in the subject are CD4+ T cells. Unlike CD8+ T cells, which recognize peptides presented by most cells of the body, CD4+ T cells recognize peptides presented by a few types of immune cells that express MHC-II proteins. These MHC-II-expressing cells, also referred to as “antigen-presenting cells” or “APCs,” include B cells, monocytes, macrophages, and dendritic cells. B cells expressing surface-bound antibody, upon interaction of the B cell receptor with antigen, can internalize the antigen-antibody complex, degrade the antigen into peptide fragments, and present the peptide fragments on MHC-II on the cell surface. Monocytes and macrophages, after phagocytosing a pathogen, cancer cell, tumor cell, or other antibody-bound cell or molecule, can present peptide fragments of the phagocytosed pathogen, cell, or molecule on cell surface MHC-II. Dendritic cells regularly sample proteins from their environment, a process called “pinocytosis,” and present peptide fragments of the sampled proteins on cell surface MHC-II. Often, dendritic cells sample proteins from one anatomical site, and migrate to the nearest lymph node (draining lymph node), where they will present peptides to circulating CD4+ T cells. The first interaction of a CD4+ T cell TCR with a cognate peptide: MHC on an APC, such as a dendritic cell in a lymph node, activates the CD4+ T cell. Subsequent recognition of peptide: MHC an APC surface by an activated CD4+ T cell causes the T cell to secrete one or more cytokines, promoting survival, activation, and/or proliferation of the APC. For B cells, competition for CD4+ T cell signaling favors B cells that produce antibodies with higher affinity. For monocytes and macrophages, signaling from CD4+ T cells promotes phagocytosis and killing of pathogens, as well as inflammation that facilitates recruitment and trafficking of other immune cells, such as innate effector cells and CD8+ T cells. One cytokine commonly secreted by both CD4+ and CD8+ T cells is IFN-γ, which has many pro-inflammatory and tumoricidal effects. IFN-γ signaling by CD4+ T cells can enhance the phagocytic ability of monocytes and macrophages, polarizing them towards a pro-inflammatory and tumoricidal phenotype by inducing the production of toxic compounds such as reactive oxygen species and lysozyme. In the tumor microenvironment specifically, IFN-γ can inhibit tumor cell proliferation, induce apoptosis of cancer cells, and hinder tumor angiogenesis (See, e.g., Castro et al., Front Immunol. 2018. 9:847).

In some embodiments, the T cells are IFN-γ+CD4+ T cells. In some embodiments, the T cells are CD8+ T cells. In some embodiments, the T cells are IFN-γ+CD8+ T cells. In some embodiments, the T cells are CD4+CD8+ T cells. In some embodiments, the T cells are IFN-γ+CD4+CD8+ T cells. In some embodiments, the T cells are NK-T cells.

In some embodiments of the methods provided herein, the method results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of the volume of a tumor in the subject. In some embodiments, the method results in a reduction of at least 10% of the volume of the tumor in the subject. In some embodiments, the method results in a reduction of at least 30% of the volume of the tumor in the subject. See, e.g., Weber, J Nucl Med. 50(Suppl 1):1S-10S.

In some embodiments, the method results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of the mass of one or more tumors in the subject. In some embodiments, the method results in a reduction of at least 30% of the mass of one or more tumors in the subject.

In some embodiments, the method results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of the mass of a primary tumor in the subject. In some embodiments, the method results in a reduction of at least 30% of the mass of a primary tumor in the subject. In some embodiments, the method results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of the volume of a primary tumor in the subject. In some embodiments, the method results in a reduction of at least 30% of the volume of a primary tumor in the subject. In some embodiments, a primary tumor refers to the largest tumor in a subject, by mass or volume. In some embodiments, a primary tumor refers to a tumor in which the largest percentage of cells are cancer cells.

In some embodiments, the method results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% in the number of circulating cancer cells in the subject. In some embodiments, the method results in a reduction of at least 10% of the number of circulating cancer cells in the subject. In some embodiments, the method results in a reduction of at least 30% of the number of circulating cancer cells in the subject. Measuring the number of circulating cancer cells in the subject may be used to evaluate the effect of a method as an addition, or alternative, to measuring the size or growth rate of a tumor. For example, cancers of hematopoietic cells such as leukemias and lymphomas may not necessarily form tumors, and so tracking the number or proportion of circulating cells that are cancer cells allows for evaluation of a method's effectiveness when measuring tumor size is not feasible. Additionally, circulating cancer cells may be cells that are shed from a tumor and may form secondary growths (metastases) at other anatomical sites. Metastases are associated with poor prognoses and increased risk of death in cancer, and so reducing the number of circulating cancer cells in a subject is a desirable outcome even when a tumor is present in the subject. See, e.g., Zhong et al., Mol Cancer. 2020. 19(1):15.

In some embodiments, the method results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of the tumor growth rate in the subject. In some embodiments, the method results in a reduction of at least 10% of the tumor growth rate in the subject. In some embodiments, the method results in a reduction of at least 30% of the tumor growth rate in the subject. See, e.g., Hather et al., Cancer Inform. 2014. 13(Suppl 4):65-72.

In some embodiments, the method results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% in the number of tumor cells in a tissue in the subject. In some embodiments, tumor cells are identified as containing a tumor antigen based on immunohistochemistry analysis. Immunohistochemistry refers to staining cells with an antibody or other reagent that selectively binds to or interacts with a target antigen, such as a tumor antigen, and visualizing stained cells, such as by microscopy. Cells that are stained are identified as containing an antigen and/or being a tumor cell, while cells that are not stained are classified as not containing an antigen and/or not being a tumor cell. In some embodiments, tumor cells are identified using histology. In some embodiments, the method results in a reduction of at least 10% in the number of tumor cells in a tissue in the subject. In some embodiments, the method results in a reduction of at least 30% of a number of tumor cells in a tissue in the subject.

In some embodiments, the method prevents or reduces the frequency of metastasis in the subject for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, at least 24 months, at least 30 months, at least 36 months, at least 42 months, at least 48 months, at least 54 months, at least 60 months, at least 66 months, at least 72 months, at least 78 months, at least 84 months, at least 90 months, at least 96 months, at least 102 months, at least 108 months, at least 114 months, or at least 120 months. Metastasis refers to the development of a secondary tumor or mass of cancer cells that is in a separate anatomical site from a primary tumor. Generally, metastasis occurs when a cancer cell is released from a tumor, circulates through the body, and begins replicating in a distinct anatomical site that is separate from the tumor that released it. In some embodiments, the method prevents or reduces the frequency of metastasis for at least 6 months. In some embodiments, the method prevents or reduces the frequency of metastasis for at least 12 months. In some embodiments, the method prevents or reduces the frequency of metastasis for at least 24 months. In some embodiments, the method prevents or reduces the frequency of metastasis for at least 36 months. In some embodiments, the method prevents or reduces the frequency of metastasis for at least 48 months. In some embodiments, the method prevents or reduces the frequency of metastasis for at least 60 months. In some embodiments, the method prevents or reduces the frequency of metastasis for at least 72 months. In some embodiments, the method prevents or reduces the frequency of metastasis for at least 84 months. In some embodiments, the method prevents or reduces the frequency of metastasis for at least 96 months. In some embodiments, the method prevents or reduces the frequency of metastasis for at least 108 months. In some embodiments, the method prevents or reduces the frequency of metastasis for at least 120 months.

In some embodiments, the method prevents the recurrence of cancer for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, at least 24 months, at least 30 months, at least 36 months, at least 42 months, at least 48 months, at least 54 months, at least 60 months, at least 66 months, at least 72 months, at least 78 months, at least 84 months, at least 90 months, at least 96 months, at least 102 months, at least 108 months, at least 114 months, or at least 120 months. In some embodiments, the method prevents the recurrence of cancer for at least 6 months. In some embodiments, the method prevents the recurrence of cancer for at least 12 months. In some embodiments, the method prevents the recurrence of cancer for at least 24 months. In some embodiments, the method prevents the recurrence of cancer for at least 36 months. In some embodiments, the method prevents the recurrence of cancer for at least 48 months. In some embodiments, the method prevents the recurrence of cancer for at least 60 months. In some embodiments, the method prevents the recurrence of cancer for at least 72 months. In some embodiments, the method prevents the recurrence of cancer for at least 84 months. In some embodiments, the method prevents the recurrence of cancer for at least 96 months. In some embodiments, the method prevents the recurrence of cancer for at least 108 months. In some embodiments, the method prevents the recurrence of cancer for at least 120 months. Recurrence of cancer can be determined by any one of multiple methods known in the art, including biopsy of an affected organ, histopathology, immunohistochemistry, flow cytometry, physical examination, imaging, and DNA or RNA sequencing.

In some embodiments of the methods provided herein, the method further comprises administering an anti-cancer agent to the subject. In some embodiments, the anti-cancer agent is an antibody or antigen-binding fragment thereof. In some embodiments, the antibody or antigen-binding fragment thereof is an immune checkpoint inhibitor. An immune checkpoint inhibitor refers to an agent that blocks the activity of one or more immune checkpoints, including PD-1, PD-L1, and CTLA-4. For example, signaling between PD-1 on one immune cell, such as a T cell, and PD-L1 on another cell, such as an antigen-presenting cell, results in the transduction of an inhibitory signal in the T cell, which hinders the anti-cancer activities of the T cell. Inhibiting PD-1 signaling by blocking PD-1, PD-L1, or both, can thus maintain the anti-cancer efficacy of T cells. In some embodiments, the antibody or antigen-binding fragment thereof is an anti-PD-1 antibody, anti-PD-L1 antibody, anti-CTLA-4 antibody, anti-TIGIT antibody, anti-TIM3 antibody, anti-LAG3 antibody, anti-CD200 antibody, or anti-CD200R antibody. In some embodiments, the anti-cancer agent is a chemotherapeutic agent. In some embodiments, the anti-cancer agent is an agonist of a positive immune checkpoint. A positive immune checkpoint refers to a receptor on a cell that, when stimulated, transduces an activating signal to the cell. In some embodiments, the positive immune checkpoint is selected from the group consisting of CD27, CD28, CD40, CD122, 4-1BB, OX40, GITR, and ICOS. In some embodiments, the positive immune checkpoint is selected from the group consisting of CD40, 4-1BB, and GITR. In some embodiments, the anti-cancer agent is an antibody or antigen-binding fragment thereof that binds to CD27, CD28, CD40, CD122, 4-1BB, OX40, GITR, or ICOS. In some embodiments, the anti-cancer agent is an antibody or antigen-binding fragment thereof that binds to CD40, 4-1BB, or GITR.

In some embodiments, the anti-cancer agent is a chemotherapeutic agent. A chemotherapeutic agent is a chemical compound useful in the treatment of cancer. Examples of chemotherapeutic agents include alkylating agents such as thiotepa and cyclophosphamide (CYTOXAN); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, tricthiylenethiophosphoramide and trimethylolomelamine; acetogenins (especially bullatacin and bullatacinonc); delta-9-tetrahydrocannabinol (dronabinol, MARINOL); beta-lapachone; lapachol; colchicines; betulinic acid; a camptothecin (including the synthetic analogue topotecan (HYCAMTIN), CPT-11 (irinotecan, CAMPTOSAR), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); cleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calichcamicin gamma1I and calicheamicin omegaI1 (See, e.g., Nicolaou et al., Angew. Chem Intl. Ed. Engl., 33:183-186 (1994)); dynemicin, including dynemicin A; an esperamicin; as well as neocarzinostatin chromophore and related chromoprotein enediyne antibiotic chromophores), aclacinomysins, actinomycin, authramycin, azascrine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN, morpholino-doxorubicin, cyanomorpholino-doxorubicin, 2-pyrrolino-doxorubicin, doxorubicin HCl liposome injection (DOXIL) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR), tegafur (UFTORAL), capecitabine (XELODA), an epothilone, and 5-fluorouracil (5-FU); folic acid analogues such as denopterin, methotrexate, pteropterin, trimetrexate; purine analogs such as fludarabinc, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurca; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesinc (ELDISINE, FILDESIN); dacarbazine; mannomustinc; mitobronitol; mitolactol; pipobroman; gacytosine; arabinoside (“Ara-C”); thiotepa; taxoids, e.g., paclitaxel (TAXOL), albumin-engineered nanoparticle formulation of paclitaxel (ABRAXANE), and doxctaxel (TAXOTERE); chloranbucil; 6-thioguanine; mercaptopurine; methotrexate; platinum analogs such as cisplatin and carboplatin; vinblastine (VELBAN); platinum; ctoposide (VP-16); ifosfamide; mitoxantrone; vincristine (ONCOVIN); oxaliplatin; leucovovin; vinorelbine (NAVELBINE); novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN) combined with 5-FU and leucovovin.

Chemotherapeutic agents also include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX tamoxifen), raloxifene (EVISTA), droloxifene, 4-hydroxytamoxifen, trioxifene, keoxifene, LY117018, onapristone, and toremifene (FARESTON); anti-progesterones; estrogen receptor down-regulators (ERDs); estrogen receptor antagonists such as fulvestrant (FASLODEX); agents that function to suppress or shut down the ovaries, for example, leutinizing hormone-releasing hormone (LHRH) agonists such as leuprolide acetate (LUPRON and ELIGARD), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE), exemestane (AROMASIN), formestanic, fadrozole, vorozole (RIVISOR), letrozole (FEMARA), and anastrozole (ARIMIDEX). In addition, such definition of chemotherapeutic agents includes bisphosphonates such as clodronate (for example, BONEFOS or OSTAC), etidronate (DIDROCAL), NE-58095,zoledronic acid/zoledronate (ZOMETA), alendronate (FOSAMAX), pamidronate (AREDIA), tiludronate (SKELID), or risedronate (ACTONEL); as well as troxacitabine (a 1,3-dioxolane nucleoside cytosine analog); anti-sense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE vaccine and gene therapy vaccines, for example, ALLOVECTIN vaccine, LEUVECTIN vaccine, and VAXID vaccine; topoisomerase 1 inhibitor (e.g., LURTOTECAN); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

In some embodiments, the method further comprises administering Fms-related tyrosine kinase 3 ligand (FLT3L) to the subject at or near the site of the tumor antigen, nucleic acid encoding the tumor antigen, or tumor antigen-containing cell. FLT3L promotes cross-presentation of antigens by dendritic cells, which is required for dendritic cells to activate CD8+ T cells. See, e.g., Kirkling et al., Cell Rep. 2018. 23(12):3658-3672. An example of a DNA sequence encoding FLT3L is given by Accession No. U04806.1, and reproduced as SEQ ID NO: 29. An example of an amino acid sequence of FLT3L is given by Accession No. P49771, and reproduced as SEQ ID NO: 30. In some embodiments, the FLT3L comprises an amino acid sequence with at least 90%, at least 95%, at least 96%, at least 97%, at least 98%, at least 99%, or up to 100% sequence identity to the amino acid sequence of SEQ ID NO: 30. In some embodiments, the FLT3L comprises the amino acid sequence of SEQ ID NO: 30. In some embodiments, the FLT3L is administered at or near the site of the tumor avatar. In some embodiments, the FLT3L is administered systemically (e.g., intravenously). In some embodiments, the FLT3L is administered prior to the generation of the tumor avatar in the subject.

In some embodiments, the method further comprises administering a Notch ligand or a nucleic acid encoding a Notch ligand to the subject at or near the site of the tumor avatar. Non-limiting examples of Notch ligands include Jagged1, Dll1, and Dll4. See, e.g., D'Souza et al., Curr Top Dev Biol. 2010. 92:73-129. In some embodiments, the Notch ligand is Jagged1. In some embodiments, the Notch ligand is Dll1. In some embodiments, the Notch ligand is Dll4. In some embodiments, the Notch ligand is administered prior to the generation of the tumor avatar in the subject.

In some embodiments, the method further comprises administering one or more chemokines or nucleic acids encoding one or more chemokines to the subject at or near the site of the tumor avatar. Chemokines are known in the art, and are a type of cytokine that induce chemotaxis in nearby responsive cells, typically white blood cells, to sites of infection. Non-limiting examples of chemokines include CCL14, CCL19, CCL20, CCL21, CCL25, CCL27, CXCL12, CXCL13, CXCL-8, CCL2, CCL3, CCL4, CCL5, CCL11, and CXCL10.

In some embodiments, the method further comprises administering an invariant natural killer T (iNK-T) cell to the subject at or near the site of the tumor avatar, wherein the iNK-T cell comprises a chimeric antigen receptor (CAR) comprising: (a) an antigen-binding domain that specifically binds to the tumor antigen; (b) a transmembrane domain; and (c) one or more intracellular signaling domains. In addition to killing tumor avatar cells expressing tumor antigens, iNK-T cells can enhance the ability of dendritic cells to process extracellular antigens and cross-present peptides on MHC-I. See, e.g., Simonetta et al., Clin Cancer Res. 2021. clincanres.1329.2021. Administration of iNK-T cells thus causes immunogenic cell death of tumor avatar cells, and facilitates cross-presentation of tumor antigens of the tumor avatar to CD8+ T cells, thereby stimulating a tumor-specific CD8+ T cell response. In some embodiments, the one or more intracellular signaling domains are selected from the group consisting of 4-1BB, CD28, ICOS, and CD3ζ. In some embodiments, the iNK-T cell is an allogeneic cell. In some embodiments, administering the iNK-T cell stimulates cross-presentation of one or more peptides of the tumor antigen by dendritic cells in the subject

### Pharmaceutical Compositions and Methods of Use

In some aspects, the present disclosure provides compositions comprising a tumor antigen, nucleic acid encoding a tumor antigen, tumor antigen-containing cell, a hyperactivating agent, a nucleic acid encoding a hyperactivating agent, and/or a sensitizing agent, for use in any of the methods provided herein. In some embodiments, the composition further comprises a pharmaceutically acceptable excipient. “Acceptable” means that the excipient (carrier) must be compatible with the active ingredient of the composition (and preferably, capable of stabilizing the active ingredient) and not deleterious to the subject to be treated. Pharmaceutically acceptable excipients, carriers, buffers, stabilisers, isotonicising agents, preservatives or antioxidants, or other materials well known to those skilled in the art. Such materials should be non-toxic and should not interfere with the efficacy of the active ingredient. The precise nature of the carrier or other material may depend on the route of administration, e.g., parenteral, intramuscular, intradermal, sublingual, buccal, ocular, intranasal, subcutaneous, intrathecal, intratumoral, oral, vaginal, or rectal. See, e.g., Remington: The Science and Practice of Pharmacy 20th Ed. (2000) Lippincott Williams and Wilkins, Ed. K. E. Hoover.

The pharmaceutical compositions to be used for in vivo administration must be sterile, with the exception of any cells, viruses, and/or viral vectors being used as hyperactivating agents or for delivery of nucleic acids and/or tumor antigens. This is readily accomplished by, for example, filtration through sterile filtration membranes. The pharmaceutical compositions described herein may be placed into a container having a sterile access port, for example, an intravenous solution bag or vial having a stopper pierceable by a hypodermic injection needle.

In other embodiments, the pharmaceutical compositions described herein can be formulated for intramuscular injection, intravenous injection, intradermal injection, subcutaneous injection, or skin scarification.

The pharmaceutical compositions described herein to be used in the present methods can comprise pharmaceutically acceptable carriers, buffer agents, excipients, salts, or stabilizers in the form of lyophilized formulations or aqueous solutions. See, e.g., Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, the pharmaceutical composition described herein comprises lipid nanoparticles which can be prepared by methods known in the art, such as described in Epstein et al., Proc Natl Acad Sci USA. 1985. 82:3688; Hwang et al. Proc Natl Acad Sci USA. 1980. 77:4030; and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Lipids used in the formulation of lipid nanoparticles for delivering nucleic acids are generally known in the art, and include ionizable amino lipids, non-cationic lipids, sterols, and polyethylene glycol-modified lipids. See, e.g., Buschmann et al., Vaccines. 2021. 9(1):65. In some embodiments, the nucleic acid is surrounded by the lipids of the lipid nanoparticle and present in the interior of the lipid nanoparticle. In some embodiments, the nucleic acid is dispersed throughout the lipids of the lipid nanoparticle. In some embodiments, the lipid nanoparticle comprises an ionizable amino lipid, a non-cationic lipid, a sterol, and/or a polyethylene glycol (PEG)-modified lipid.

In other embodiments, the pharmaceutical composition described herein can be formulated in sustained-release format. Suitable examples of sustained-release preparations include semipermeable matrices of solid hydrophobic polymers containing the nucleic acids, hyperactivating agents, and/or sensitizing agents, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(-)-3-hydroxybutyric acid.

Suitable surface-active agents include, in particular, non-ionic agents, such as polyoxyethylenesorbitans (e.g., TWEEN™ 20, 40, 60, 80 or 85) and other sorbitans (e.g., SPAN™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable emulsions may be prepared using commercially available fat emulsions, such as INTRALIPID™, LIPOSYN™, INFONUTROL™, LIPOFUNDIN™ and LIPIPHYSAN™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets having a suitable size and can have a pH in the range of 5.5 to 8.0.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

In some embodiments of the methods provided herein, the subject is a human. In some embodiments, the subject is an animal. In some embodiments, the animal is a research animal. In some embodiments, the animal is a domesticated animal. In some embodiments, the animal is a rodent. In some embodiments, the rodent is a mouse, rat, guinea pig, chinchilla, or hamster. In some embodiments, the animal is a dog, cat, rabbit, guinea pig, hamster, or ferret. In some embodiments, the animal is a bovine, swine, llama, alpaca, shecp, or goat. In some embodiments, the subject has or is at risk of developing cancer. In some embodiments, the cancer is selected from the group consisting of melanoma, squamous cell cancer, small-cell lung cancer, non-small cell lung cancer, lung cell adenocarcinoma, squamous lung cell carcinoma, peritoneal cancer, hepatocellular cancer, gastrointestinal cancer, esophageal cancer, stomach cancer, pancreatic cancer, glioblastoma, cervical cancer, ovarian cancer, liver cancer, bladder cancer, hepatoma, breast cancer, colon cancer, colorectal cancer, endometrial carcinoma, uterine carcinoma, salivary gland carcinoma, kidney cancer, prostate cancer, vulval cancer, thyroid cancer, hepatic carcinoma, gastric cancer, head-and-neck cancer, leukemia, and lymphoma.

In some embodiments, the composition is to be stored below 50° C., below 40° C., below 30° C., below 20° C., below 10° C., below 0° C., below −10° C., below −20° C., below −30° C., below −40° C., below −50° C., below −60° C., below −70° C., or below −80° C., such that the nucleic acid(s), hyperactivating agent(s), and/or sensitizing agent(s) are relatively stable over time.

### Methods of Administration

Some aspects of the present disclosure relate to methods of generating a tumor avatar in a subject by administering a tumor antigen, nucleic acid encoding a tumor antigen, or tumor antigen-containing cell to the subject, and causing immunogenic cell death and generating hyperactivated dendritic cells in the subject. Hyperactivated dendritic cells may be generated in a subject by any one of multiple methods described herein. Non-limiting examples of methods of generating hyperactivate dendritic cells include administering a hyperactivating agent or a nucleic acid encoding the hyperactivating agent, administering an oxidizing agent, or administering a sensitizing agent to the subject and then exposing the sensitizing agent to an energy source.

Any method of administering a nucleic acid, protein, or cell can be used to administer a nucleic acid, tumor antigen, tumor antigen-containing cell, dendritic cell, and/or hyperactivating agent to a subject. In some embodiments of the methods provided herein. tumor antigens, nucleic acids, tumor antigen-containing cells and/or hyperactivating agents are administered to a subject by injection. Injection refers to the introduction of a liquid, such as a liquid containing a nucleic acid, into a subject using a syringe and a needle. In some embodiments, the nucleic acids and/or hyperactivating agents are administered by hydrodynamic injection. Hydrodynamic injection refers to the injection of a nucleic acid directly into the bloodstream of a subject. The resulting increase in hydrodynamic pressure in the bloodstream increases the permeability of cell membranes and may also form pores in membranes, facilitating entry of the injected nucleic acid into cells. Delivery of nucleic acids by hydrodynamic injection efficiently introduces nucleic acids into cells of a subject without the need for viral vector-based gene delivery. See, e.g., Suda et al. Mol Ther. 2007. 15(12):2063-2069. In some embodiments, the tumor antigens, nucleic acids, tumor antigen-containing cells and/or hyperactivating agents are administered by jet injection. Jet injection utilizes high-pressure air to force small volumes of nucleic acids into a target tissue. The high pressure of jet injection allows nucleic acids to be delivered deep below the skin, with the applied pressure increasing membrane permeability of cells in the target tissue, facilitating entry of the injected nucleic acids into cells. See, e.g., Walther et al., Mol Biotechnol. 2004. 28(2):121-128. In some embodiments, the tumor antigens, nucleic acids, tumor antigen-containing cells, and/or hyperactivating agents are administered by in vivo electroporation. In vivo electroporation is the process of introducing nucleic acids or other molecules into a cell of a subject using a pulse of electricity, which promote passage of the nucleic acids or other molecules through the cell membrane and/or cell wall. See, e.g., Somiari et al., Molecular Therapy., 2000. 2(3):178-187. The nucleic acid or molecule to be delivered is administered to the subject, such as by injection, and a pulse of electricity is applied to the injection site, whereby the electricity promotes entry of the nucleic acid into cells at the site of administration. In some embodiments, the nucleic acid is administered with other elements, such as buffers and/or excipients, that increase the efficiency of electroporation.

In some aspects, the present disclosure provides a method of administering to a subject any of the tumor antigens, nucleic acids, tumor antigen-containing cells, hyperactivating agents, lipid nanoparticles, cells, compositions, or pharmaceutical compositions provided herein. In some embodiments, the subject is a human. In some embodiments, the administration is parenteral, intramuscular, intradermal, sublingual, buccal, ocular, intranasal, subcutaneous, intrathecal, intratumoral, oral, vaginal, or rectal. In some embodiments, the nucleic acids and/or hyperactivating agents are administered intramuscularly. In some embodiments, the nucleic acids and/or hyperactivating agents are administered intradermally. In some embodiments, the nucleic acids and/or hyperactivating agents are administered subcutaneously. In some embodiments, the nucleic acids and/or hyperactivating agents are administered intravenously.

In some embodiments of the methods provided herein, the step of causing cell death and/or generating hyperactivated dendritic cells is performed more than once. In some embodiments, the step of causing cell death and/or generating hyperactivated dendritic cells is performed 2, 3, 4, 5, 6, 7, 8, 9, 10, 11, 12, 13, 14, 15, 16, 17, 18, 19, 20, or more times. In some embodiments, the same method of causing cell death and/or generating hyperactivated dendritic cells is performed multiple times. In some embodiments, the steps of causing cell death and/or generating hyperactivated dendritic cells is performed multiple times using different methods.

In some embodiments of the methods provided herein, the tumor antigen, nucleic acid, and/or tumor antigen-containing cell are administered more than once. In some embodiments, the same tumor antigen, nucleic acid, and/or tumor antigen-containing cell are administered multiple times. In some embodiments, different administrations comprise administering different tumor antigen, nucleic acid, and/or tumor antigen-containing cell. In some embodiments, nucleic acids from the tumor and/or subject are sequenced before each administration of tumor antigen, nucleic acid, and/or tumor antigen-containing cells.

In some embodiments of the methods provided herein, the step of causing cell death and/or the step of generating hyperactivated dendritic cells is performed between 0 and 96 hours after the tumor antigen, nucleic acid encoding the tumor antigen, or tumor antigen-containing cell is administered to the subject. In some embodiments, the step of causing cell death and/or the step of generating hyperactivated dendritic cells is performed between 0 and 168, 0 and 144, 0 and 120, 0 and 96, 0 and 72 hours, between 0 and 48 hours, between 0 and 24 hours, or between 0 and 12 hours after the tumor antigen, nucleic acid encoding the tumor antigen, or tumor antigen-containing cell is administered to the subject.

In some embodiments, the step of causing cell death and/or the step of generating hyperactivated dendritic cells is performed between 4 and 96 hours after the tumor antigen, nucleic acid encoding the tumor antigen, or tumor antigen-containing cell is administered to the subject. In some embodiments, the step of causing cell death and/or generating hyperactivated dendritic cells is performed between 4-8, 8-12, 12-16, 16-20, 20-24, 24-36, 36-48, 48-60, 60-72, 72-84, or 84-96 hours after the tumor antigen, nucleic acid encoding the tumor antigen, or tumor antigen-containing cell is administered to the subject.

In some embodiments, the tumor antigen, nucleic acid encoding the tumor antigen, or tumor antigen-containing cell is administered to the subject before the hyperactivated dendritic cells. In some embodiments, the tumor antigen, nucleic acid encoding the tumor antigen, or tumor antigen-containing cell is administered to the subject after the hyperactivated dendritic cells. In some embodiments, the step of generating hyperactivated dendritic cells is performed at about the same time the tumor antigen, nucleic acid encoding the tumor antigen, or tumor antigen-containing cell is administered to the subject. A first and second composition are administered at about the same time if the time between the administration of the first composition and the administration of the second composition is up to and including, but not greater than, 24 hours. In some embodiments, a hyperactivating agent, nucleic acid encoding a hyperactivating agent, and/or sensitizing agent for generating hyperactivated dendritic cells is administered in the same composition as the nucleic acid encoding the tumor antigen. In some embodiments, the nucleic acid encoding the tumor antigen is administered in a separate composition from a hyperactivating agent, nucleic acid encoding a hyperactivating agent, and/or sensitizing agent for generating hyperactivated dendritic cells.

In some aspects, the present disclosure provides a kit comprising one or more of the tumor antigens, nucleic acids, tumor antigen-containing cells, hyperactivating agents, and/or sensitizing agents for use in any of the methods described herein. In some embodiments, the kit comprises a nucleic acid encoding a tumor antigen and a hyperactivating agent. In some embodiments, the kit comprises a nucleic acid encoding a tumor antigen and a nucleic acid encoding a tumor antigen. In some embodiments, the kit comprises a nucleic acid encoding a tumor antigen, and a sensitizing agent. In some embodiments, the kit is to be stored below 50° C. below 40° C., below 30° C., below 20° C., below 10° C., below 0° C., below −10° C., below −20° C. below −30° C., below −40° C., below −50° C., below −60° C., below −70° C., or below −80° C., such that the nucleic acids are relatively stable over time.

In some aspects, the present disclosure provides a kit comprising any of the pharmaceutical compositions provided herein and a delivery device. A delivery device refers to machine or apparatus suitable for administering a composition to a subject, such as a syringe or needle. In some embodiments, the delivery device comprises a hypodermic needle. In some embodiments, the delivery device comprises a jet injector. In some embodiments, the kit is to be stored below 50° C., below 40° C., below 30° C., below 20° C., below 10° C., below 0° C. below −10° C. below −20° C., below −30° C., below −40° C., below −50° C., below −60° C. below −70° C., or below −80° C., such that the nucleic acids of the pharmaceutical composition are relatively stable over time.

### EXAMPLES

In order that the disclosure may be more fully understood, the following examples are set forth. The examples are offered to illustrate the methods, pharmaceutical compositions, and kits provided herein and are not to be construed in any way as limiting their scope.

### Example 1: Tumor Avatar Generation

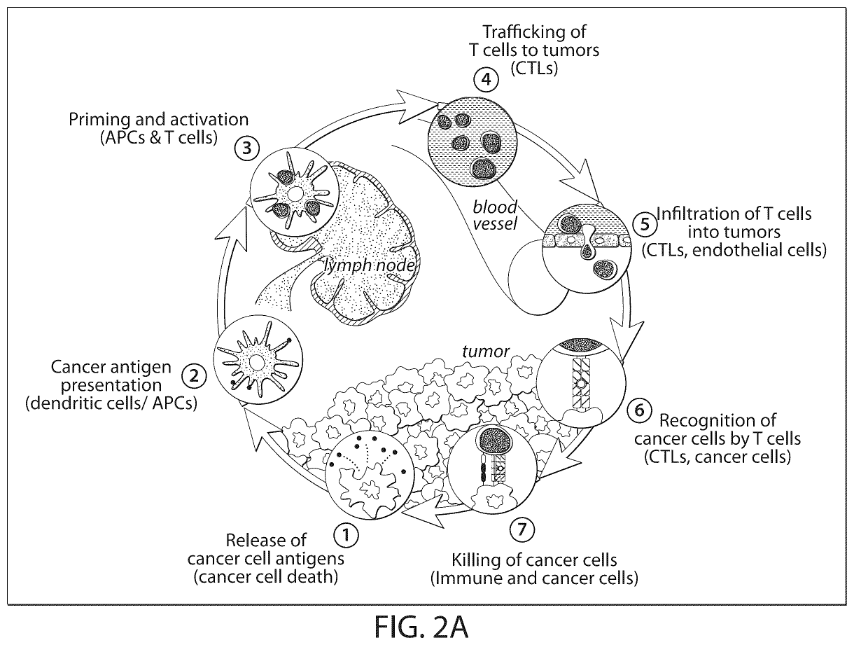

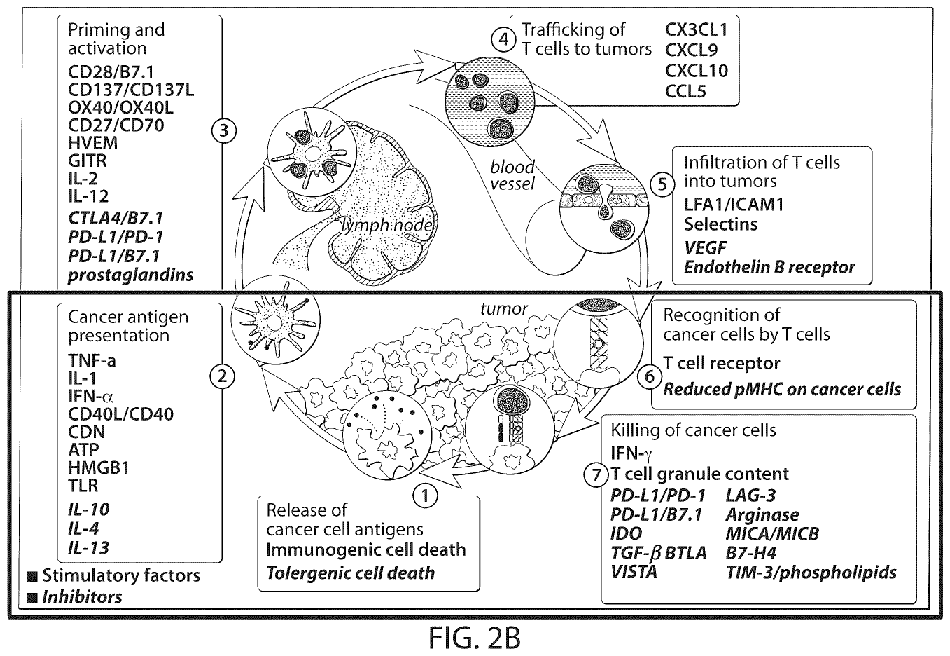

Methods have been developed to mimic the natural cycle of immunity for use in cancer immunotherapy. This natural cycle involves the steps of 1) release of cancer antigens after cancer cell death; 2) presentation of cancer antigens (tumor antigens) by dendritic cells and/or other antigen-presenting cells (APCs); 3) priming and activation of T cells in the lymph node; 4) trafficking of T cells to tumors; 5) infiltration of T cells into tumors; 6) recognition of cancer cells by T cells; and 7) killing of cancer cells by T cells (FIG. 2A). See, e.g., Chen and Mellman. Immunity. 2013. 39(1):1-10.

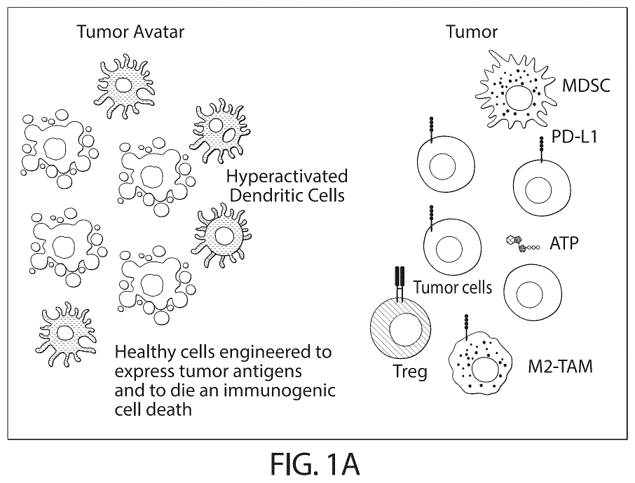

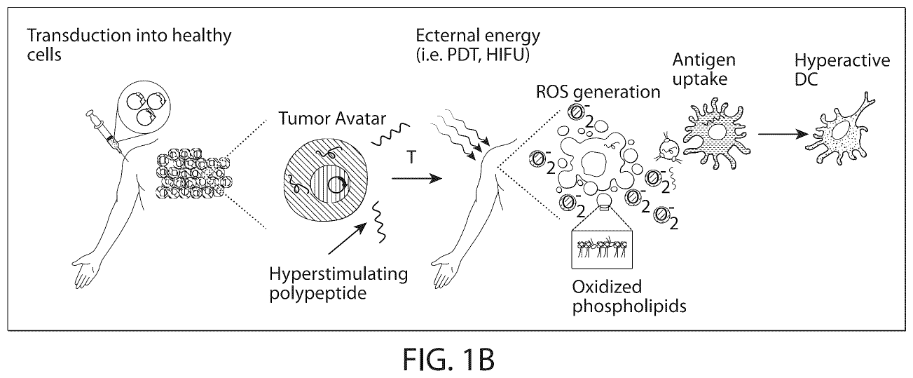

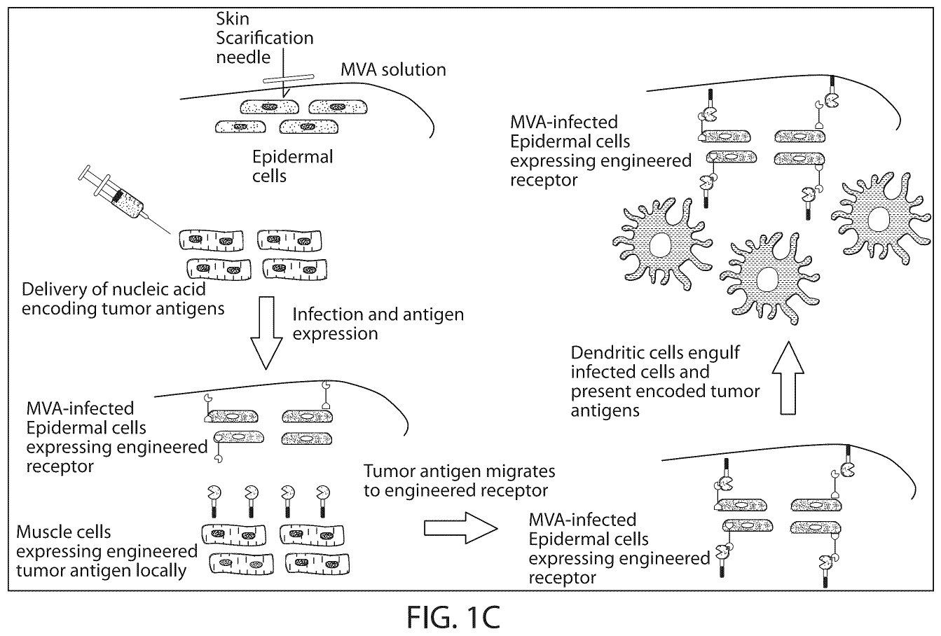

Anti-tumor vaccines elicit anti-tumor immune responses by generating tumor avatars in a subject at a site that is distant from the anatomical location of the tumor. A tumor avatar refers to a collection of cells in a subject that mimics the antigenic environment of a tumor, but is located outside of a tumor in a subject, and is produced by generating tumor antigen-containing cells at a site in the subject that is separate from a tumor. Tumor avatars are generated by the administration of tumor antigens that bind to or are internalized by cells at an anatomical location that is separate from the tumor, nucleic acids encoding tumor antigens that are inserted into cells at an anatomical location that is separate from the tumor, and/or tumor antigen-containing cells injected into an anatomical location that is separate from the tumor. Cells of the tumor avatar contain tumor antigens, but because the tumor avatar does not contain cancerous cells or regulatory cells that are present in tumors, the tumor avatar microenvironment is not immunosuppressive in the same way that the tumor microenvironment is (FIG. 1A). The lack of an immunosuppressive environment in the tumor avatar microenvironment allows for more efficient sampling of tumor antigens and presentation of peptide fragments of tumor antigens to activate a robust anti-tumor immune response.

Intentional killing of cells of the tumor avatar causes the release of the expressed tumor antigens and immunostimulatory signals that facilitate inflammation, recruitment of innate cells, and presentation of peptide fragments of the tumor antigens to cells of the adaptive immune system. Cell death is accomplished by one of multiple methods, including the administration of a virus or virus-like particle, administration of a sensitizing agent followed by exposure to an energy source, and/or mechanical stress at the site of antigen administration (FIGS. 1B-1C). One class of immunostimulatory signals released by dead or dying cells, oxidation products of 1-palmitoyl-2-arachidonyl-sn-glycero-3-phosphorylcholine, is directly administered to activate immune cells without directly causing cell death. Inducing cell death and/or administering hyperactivating agents such as oxidized phospholipids generates hyperactivated dendritic cells, which secrete IL-1β and IL-18. Dendritic cells are recruited to the tumor avatar, where they become hyperactivated, and sample tumor antigens from the tumor avatar microenvironment. Hyperactivated dendritic cells readily migrate to a nearby lymph node (draining lymph node), where they present peptide fragments of the sampled tumor antigens to CD4+ and CD8+ T cells or transfer antigens to lymph node-resident dendritic cells for presentation to CD4+ and CD8+ T cells.

The tumor avatar can be further modified to support an immune response through the expression of immune adjuvants, cytokines, and/or chemokines. Adjuvants, cytokines, and/or chemokines are delivered to the subject directly, or encoded by nucleic acids administered with the tumor antigen, nucleic acid encoding the tumor antigen, or tumor antigen-containing cells.

### Example 2: Ex Vivo Generation of Tumor Avatar Cells

Tumor avatar cells (containing a nucleic acid library encoding one or more tumor antigens) are be generated outside the body, and then administered in an immunogenic form at a site different from the tumor site. Additionally, allogeneic cells may be used as the recipients of the nucleic acid library. A human cell line (e.g., HEK293 or K562 cells) is used to create an allogeneic, antigen-bearing ‘avatar’ of tumor cells—Avatar^{Ag} cells expressing the same antigens as expressed in the tumor cells. Using a cell line for transfection eliminates the need to transfect patient-derived samples, which can be affected by patient-to-patient variability both in terms of cell numbers as well as cell quality. Moreover, such a cell line can be readily expanded, before or after transfection. Such Avatar^{Ag} cells may be used, for example, in two different methods to immunize patients.

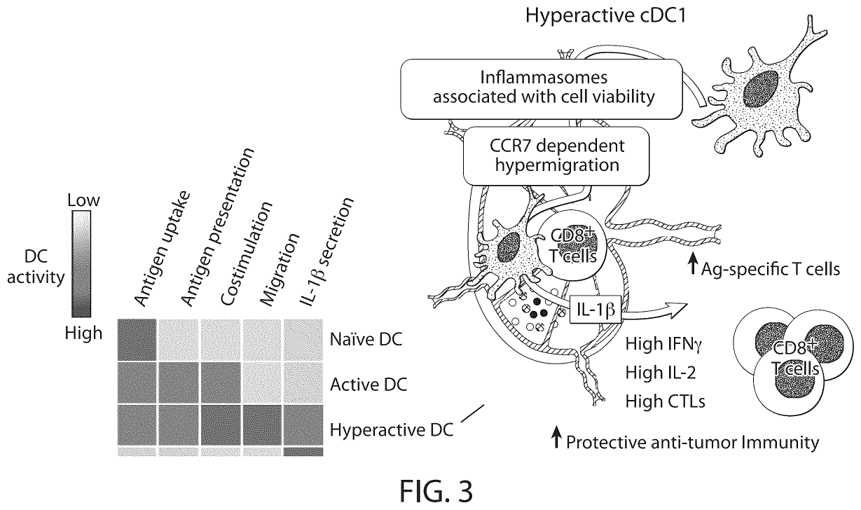

In a first method, hyperactivated dendritic cells (FIG. 3, adapted from Zhivaki et al. 2020. Cell Reports. 33(7):108381) are created ex vivo and loaded with tumor cell lysates, such as those prepared from Avatar^{Ag} cells, or loaded with Avatar^{Ag} cells themselves.

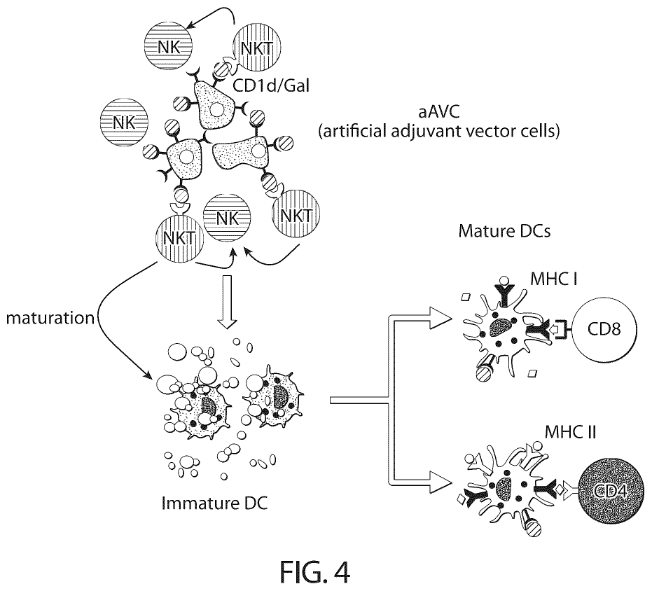

In a second method, cells expressing a tumor antigen and the CD1d surface recptor loaded with the small molecule stimulant α-galactosylceramide (αGalCer) stimulates a strong innate response, followed by adaptive immunity targeted at the carried tumor antigen (FIG. 4, adapted from Fuji et al., 2022. Cancer Sci. 113(3):864-874). Initially tested with αGalCer-loaded dendritic cells or tumor cells, a translatable approach was developed based on the use of an allogeneic murine or human cell line containing a esophageal squamous cell carcinoma antigen NY-ESO-1 and expressing CD1d for aGalCer loading. Such artificial Antigen Vector Cells (aAVCs) stimulate strong adaptive immunity in multiple murine tumor models, in murine models of infectious disease, and in companion dogs. See, e.g., Fuji et al., 2022. Cancer Sci. 113(3):864-874. Avatar^{Ag} cells expressing CD1d and loaded with αGC can be prepared and used as a cellular vaccine, potentially following lethal irradiation of the cells.

As described above, the Avatar^{Ag} cells or the cell lysate from such cells deliver the antigens to dendritic cells, but do not present the antigens directly. Thus, an allogeneic cell line can be utilized to prepare these cells, as HLA-matching to the patient is not required. Furthermore, use of a cell line allows greater control and standardization of transfection and growth, eliminating patient-to-patient variability of autologous cells. Human HEK293 cells are a human cell line established from non-tumorous human embryonic kidney cells, which underwent subsequent immortalization. Irradiated HEK293 cells have been used in humans as an alloantigen and in companion dogs as a xenoantigen cell vector without any demonstrable safety concerns and thus represent suitable cellular targets for this application. Human K562 cells, derived from lymphoblasts of a patient with chronic myelogenous leukemia, have also been safely used in humans following irradiation.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. “Or” may be used interchangeably with “and/or.” The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

What is claimed is:

1. A method of eliciting an immune response to a tumor antigen in a subject, the method comprising:
(a) administering to the subject a tumor antigen, a nucleic acid encoding a tumor antigen, or a tumor antigen-containing cell at a site that is separate from a tumor in the subject, whereby the administering generates a tumor avatar in the subject at a site that is separate from a tumor in the subject, wherein the tumor avatar comprises a population of tumor antigen-containing cells; and
(b) causing death of one or more cells of the tumor avatar, whereby cell death stimulates an inflammatory response;
whereby an immune response specific to the tumor antigen is generated in the subject.

2. The method of claim 1, wherein the method comprises:
(i) generating tumor antigen-containing cells in vitro or ex vivo by delivering a tumor antigen to an isolated population of cells, whereby one or more cells acquires the tumor antigen, thereby producing a tumor antigen-containing cell; and
(ii) administering the tumor antigen-containing cells to the subject at a site that is separate from a tumor in the subject.

3. The method of claim 1, wherein the method comprises:
(i) generating tumor antigen-containing cells in vitro or ex vivo by delivering a nucleic acid encoding a tumor antigen to an isolated population of cells, whereby one or more cells acquires and expresses the tumor antigen, thereby producing a tumor antigen-containing cell; and
(ii) administering the tumor antigen-containing cells to the subject at a site that is separate from a tumor in the subject.

4. The method of any one of claims 1-3, wherein the method further comprises:
(c) generating hyperactivated dendritic cells in the subject, wherein the hyperactivated dendritic cells secrete IL-1β and/or IL-18.

5. The method of claim 4, wherein the step of generating hyperactivated dendritic cells in the subject comprises:
(i) generating hyperactivated dendritic cells in vitro or ex vivo; and
(ii) administering hyperactivated dendritic cells to the subject at or near the tumor avatar.

6. The method of claim 4 or 5, wherein the step of generating hyperactivated dendritic cells in vitro or ex vivo comprises delivering a hyperactivating agent or a nucleic acid encoding a hyperactivating agent to an isolated population of dendritic cells.

7. The method of any one of claims 4-6, wherein the hyperactivated dendritic cells are autologous dendritic cells.

8. The method of claim 5 or 6, wherein the hyperactivated dendritic cells are allogeneic dendritic cells.

9. The method of any one of claims 1-3, wherein the step of generating hyperactivated dendritic cells comprises administering a hyperactivating agent or a nucleic acid encoding a hyperactivating agent to the subject at or near the tumor avatar.

10. The method of any one of claims 4-9, wherein the hyperactivating agent is an oxidized phospholipid, a LysM-containing protein, an HMGB1, a granzyme, an IL-1β-containing protein, a Toll-like receptor-stimulating protein, a virus, or a virus-like particle.

11. The method of any one of claims 4-10, wherein the hyperactivating agent is an oxidized phospholipid.

12. The method of claim 11, wherein the oxidized phospholipid is an oxidation product of 1-palmitoyl-2-arachidonyl-sn-glycero-3-phosphorylcholine (PAPC).

13. The method of claim 12, wherein the oxidized phospholipid is 1-palmitoyl-2-glutaryl-sn-glycero-3-phosphocholine (PGPC).

14. The method of any one of claims 4-10, wherein the hyperactivating agent is a LysM-containing protein.

15. The method of claim 14, wherein the LysM-containing protein comprises an amino acid sequence with at least 90%, at least 95%, at least 96%, at least 97%, at least 98%, at least 99%, or up to 100% sequence identity to the amino acid sequence of SEQ ID NO: 5.

16. The method of any one of claims 4-10, wherein the hyperactivating agent is an HMGB1.

17. The method of claim 16, wherein the HMGB1 comprises an amino acid sequence with at least 90%, at least 95%, at least 96%, at least 97%, at least 98%, at least 99%, or up to 100% sequence identity to the amino acid sequence of SEQ ID NO: 11.

18. The method of any one of claims 4-10, wherein the hyperactivating agent is a granzyme.

19. The method of claim 18, wherein the granzyme comprises an amino acid sequence with at least 90%, at least 95%, at least 96%, at least 97%, at least 98%, at least 99%, or up to 100% sequence identity to the amino acid sequence of SEQ ID NO: 12.

20. The method of any one of claims 4-10, wherein the hyperactivating agent is a fusion protein comprising a Toll-like receptor-stimulating domain and a glucose-regulated protein (grp170) domain.

21. The method of claim 20, wherein the Toll-like receptor-stimulating domain is selected from the group consisting of a heat shock protein domain, tRNA synthetase domain, a fibrinogen domain, a profilin domain, or a flagellin domain.

22. The method of claim 20 or 21, wherein the fusion protein is a flagellin-grp170 protein comprising amino acid sequences with at least 90%, at least 95%, at least 96%, at least 97%, at least 98%, at least 99%, or up to 100% sequence identity to each of the amino sequences of SEQ ID NO: 10, 17, and 18.

23. The method of claim 20 or 21, wherein the fusion protein is a tRNA synthetase-grp170 protein comprising:
(a) a tRNA synthetase domain comprising an amino acid sequence with at least 90%, at least 95%, at least 96%, at least 97%, at least 98%, at least 99%, or up to 100% sequence identity to the amino acid sequence of SEQ ID NO: 8 or SEQ ID NO: 9; and
(b) a grp170 domain an amino acid sequence with at least 90%, at least 95%, at least 96%, at least 97%, at least 98%, at least 99%, or up to 100% sequence identity to the amino acid sequence of SEQ ID NO: 10.

24. The method of any one of claims 4-10, wherein the hyperactivating agent is a cowpea mosaic virus (CPMV) protein, CPMV virus particle, or CPMV virus-like particle.

25. The method of claim 24, wherein the CPMV protein is a small coat protein or a large coat protein.

26. The method of claim 24 or 25, wherein the CPMV protein is a CPMV small coat protein or CPMV large coat protein, wherein the CPMV protein comprises an amino acid sequence with at least 90%, at least 95%, at least 96%, at least 97%, at least 98%, at least 99%, or up to 100% sequence identity to the amino acid sequence SEQ ID NO: 21 or SEQ ID NO: 22.

27. The method of any one of claims 4-10, wherein the hyperactivating agent is a fusion protein comprising a cytokine domain and an anchoring domain, wherein the cytokine domain comprises an amino acid sequence selected from the group consisting of GM-CSF, MIP-1α, IL-1β, IL-6, IL-8, IL-12, IL-17, IL-18, IP-10, IFN-γ, and TNF-α.

28. The method of claim 27, wherein the anchoring domain is an antibody or antigen-binding fragment thereof.

29. The method of claim 28, wherein the antibody or antigen-binding fragment thereof binds to an antigen selected from the group consisting of fibronectin extra domain A, fibronectin extra domain B, collagen, and elastin

30. The method of claim 28, wherein the antibody or antigen-binding fragment thereof binds to a cell adhesion molecule.

31. The method of claim 27, wherein the anchoring domain is a collagen-binding domain.

32. The method of claim 27 or 31, wherein the anchoring domain is a lumican domain or an A3 domain of von Willebrand Factor.

33. The method of claim 32, wherein the lumican domain comprises at least 90%, at least 95%, at least 96%, at least 97%, at least 98%, at least 99%, or up to 100% sequence identity to the amino acid sequence of SEQ ID NO: 26.

34. The method of any one of claims 27-33, wherein the cytokine domain is an IL-1β domain comprising at least 90%, at least 95%, at least 96%, at least 97%, at least 98%, at least 99%, or up to 100% sequence identity to the amino acid sequence of SEQ ID NO: 24.

35. The method of any one of claims 4-10, wherein the hyperactivating agent is a virus or a virus-like particle.

36. The method of claim 35, wherein the virus is vaccinia virus, modified vaccinia Ankara (MVA).

37. The method of claim 35 or 36, wherein the virus comprises a nucleic acid encoding an engineered receptor, whereby the engineered receptor is expressed on the surface of a cell infected by the virus, wherein the engineered receptor is capable of binding a tumor antigen.

38. The method of claim 37, wherein the engineered receptor further comprises an intracellular domain of CLEC9A.

39. The method of claim 38, wherein the intracellular domain of CLEC9A comprises an amino acid sequence with at least 90%, at least 95%, at least 96%, at least 97%, at least 98%, at least 99%, or up to 100% sequence identity to the amino acid sequence of SEQ ID NO: 4.

40. The method of any one of claims 37-39, wherein the tumor antigen further comprises a ligand that is capable of binding to the engineered receptor.

41. The method of any one of claims 35-40, wherein the virus or virus-like particle is administered to the skin of the subject.

42. The method of claim 41, wherein the virus or virus-like particle is administered by skin scarification.

43. The method of any one of claims 4-42, wherein the hyperactivating agent further comprises the tumor antigen or a nucleic acid encoding the tumor antigen.

44. The method of any one of claims 1-43, wherein the step of causing cell death comprises delivering a cytotoxic agent to the subject at or near the tumor avatar.

45. The method of claim 44, wherein the cytotoxic agent is a chemotherapeutic agent.

46. The method of claim 44 or 45, wherein the cytotoxic agent is squaric acid, epirubicin, bleomycin, bortezomib, cyclophosphamide, doxorubicin, idarubicin, mitoxantrone, or oxaliplatin.

47. The method of any one of claims 1-46, wherein the step of causing cell death comprises:
(a) administering a sensitizing agent or a nucleic acid encoding a sensitizing agent to the subject at or near the tumor avatar; and
(b) exposing the sensitizing agent to an energy source.

48. The method of claim 47, wherein the sensitizing agent is a photosensitizing agent, and the energy source is light.

49. The method of claim 48, wherein the light is ultraviolet, visible, or infrared light.

50. The method of claim 48 or 49, wherein the photosensitizing agent is encoded by a nucleic acid, wherein the nucleic acid is administered to the subject.

51. The method of any one of claims 47-50, wherein the photosensitizing agent is a mini singlet oxygen generator (miniSOG).

52. The method of claim 47, wherein the sensitizing agent is an acoustic sensitizer, and the energy source is ultrasound.

53. The method of any one of claims 1-52, wherein the step of causing cell death comprises administering a nucleic acid encoding a surface antigen to the subject at or near the site of the tumor avatar, and administering to the subject an antibody specific to the surface antigen.

54. The method of claim 53, wherein binding of the antibody to the antigen forms an immune complex that is capable of being bound by dendritic cells and/or neutrophils.

55. The method of claim 53, wherein the surface antigen is CD20, and the antibody is an anti-CD20 antibody.

56. The method of claim 53, wherein the surface antigen is epidermal growth factor receptor (EGFR), and the antibody is an anti-EGFR antibody.

57. The method of any one of claims 53-56, wherein the surface antigen and one or more tumor antigens are encoded by the same nucleic acid.

58. The method of any one of claims 1-57, wherein the step of causing cell death in the subject comprises administering an invariant natural killer T (iNK-T) cell to the subject at or near the site of the tumor avatar, wherein the iNK-T cell comprises a chimeric antigen receptor (CAR) comprising:
(i) an antigen-binding domain that specifically binds to the tumor antigen;
(ii) a transmembrane domain; and
(iii) one or more intracellular signaling domains.

59. The method of claim 58, wherein the one or more intracellular signaling domains are selected from the group consisting of 4-1BB, CD28, ICOS, and CD3ζ.

60. The method of claim 58 or 59, wherein the iNK-T cell is an allogeneic cell.

61. The method of any one of claims 58-60, wherein administering the iNK-T cell stimulates cross-presentation of the tumor antigen by a dendritic cell in the subject.

62. The method of any one of claims 1-61, wherein the tumor antigen-containing cell comprises CD1d bound to an antigen capable of being recognized by an invariant natural killer T (iNK-T) cell.

63. The method of claim 62, wherein the antigen capable of being recognized by the iNK-T cell is selected from the group consisting of α-galactosylceramide (αGalCer), α-galactosyldiacylglycerol (αGalDag), α-glucuronsylceramide (α-GLcACer), α-galacturonosylceramide (α-GalACer), phosphatidyl-myo-inositol mannoside (PIM2), β-glucosylceramide (βGlcCer), plasmalogen lysophosphatidylethanolamine (plasmalogen lysoPE), isoglobotrihexosylceramide (iGb3), and α-glucosyldiacyglycerol (αGlcDAG).

64. The method of any one of claims 1-63, wherein the tumor antigen-containing cell is an allogeneic cell.

65. The method of claim 64, wherein the tumor antigen-containing cell is a 786-O, A549, ACHN, BCP-1, BOSC23, BT-20, BT-549, BxPC-3, Caco-2, Caki-1, Calu-3, CCRF-CEM, DAOY, DU145, H295R, H1299, HaCaT, HAP1, HCC-2998, HCT116, HEK293, HEL, HeLa, HepG2, HL-60, Hs 578T, HT-29, HT1080, Huh7, HUVEC, Jurkat, JY, K562, KBM-7, KM12, LAPC4, LNCaP, LOX-IMVI, M14, MCF-7, MCF-7/ADR-RES, MDA-MB-231, MDA-MB-435, MDA-MB-453, MDA-MB-468, MDA-N, MIA PaCa-2, MOLT-4, MRC-5, NCI-60, NCI-H23, NCI-H226, NCI-H460, NCI/ADR-RES, NK-92, NTERA-2, OVCAR-3, OVCAR-8, PANC-1, PC3, Raji, RPMI-7951, Saos-2, SF-268, SF-539, SH-SY5Y, SK-MEL-5, SK-MEL-28, SK-OV-3, SKBR3, SNB-19, SW-620, T-47D, T98G, TF-1, THP-1, U87, U251, U937, VCaP, VG-1, WI-38, and ZR-75-1 cell.

66. The method of any one of claims 1-65, wherein the tumor antigen, nucleic acid encoding the tumor antigen, or tumor antigen-containing cell is administered by injection.

67. The method of claim 66, wherein the tumor antigen, nucleic acid encoding the tumor antigen, or tumor antigen-containing cell is administered by hydrodynamic injection or jet injection.

68. The method of any one of claims 1-67, wherein the tumor antigen or nucleic acid encoding the tumor antigen is introduced into a cell in the subject by in vivo electroporation.

69. The method of any one of claims 1-68, wherein the tumor antigen, nucleic acid encoding the tumor antigen, or tumor antigen-containing cell is administered intramuscularly, intradermally, or subcutaneously.

70. The method of any one of claims 1-69, wherein the tumor antigen, nucleic acid encoding the tumor antigen, or tumor antigen-containing cell is administered to the subject more than once.

71. The method of any one of claims 1-70, wherein the step of causing cell death is performed between 0 and 168, 0 and 144, between 0 and 120, 0 and 96, between 0 and 72 hours, between 0 and 48 hours, between 0 and 36 hours, between 0 and 24 hours, or between 0 and 12 hours after the tumor antigen, nucleic acid encoding the tumor antigen, or tumor antigen-containing cell is administered to the subject.

72. The method of any one of claims 1-71, wherein the step of causing cell death is performed between 4-96 hours after the tumor antigen, nucleic acid encoding the tumor antigen, or tumor antigen-containing cell is administered to the subject.

73. The method of any one of claims 1-71, wherein the step of causing cell death is performed at about the same time the tumor antigen or tumor antigen-containing cell is administered to the subject.

74. The method of any one of claims 1-73, wherein the nucleic acid encodes more than one tumor antigen.

75. The method of claim 74, wherein the nucleic acid encoding the tumor antigen encodes 2-20 tumor antigens.

76. The method of claim 74 or 75, wherein the nucleic acid encoding the tumor antigen encodes 5-10 tumor antigens.

77. The method of any one of claims 1-73, wherein the nucleic acid encoding the tumor antigen encodes 1, 2, 3, 4, 5, 6, 7, 8, 9, or 10 tumor antigens.

78. The method of any one of claims 1-77, wherein the nucleic acid encoding the tumor antigen is a DNA.

79. The method of any one of claims 1-77, wherein the nucleic acid encoding the tumor antigen is an RNA.

80. The method of claim 79, wherein the RNA is an mRNA.

81. The method of any one of claims 1-80, wherein the nucleic acid encoding the tumor antigen is comprised in a nucleic acid library that is administered to the subject, wherein each nucleic acid of the library encodes one or more tumor antigens.

82. The method of claim 81, wherein the nucleic acid library encodes 2 to 1,000, 2 to 500, 2 to 250, 2 to 100, or 2 to 50 tumor antigens.

83. The method of claim 81 or 82, wherein the nucleic acid library encodes 20-100 tumor antigens.

84. The method of any one of claims 81-83, wherein the nucleic acid library encodes at least 50%, at least 60%, at least 70%, at least 80%, at least 90%, at least 95%, at least 97%, at least 98%, at least 99%, or up to 100% of the tumor antigens present in a tumor cell in the subject.

85. The method of any one of claims 81-84, wherein one or more of the nucleic acids of the library are DNAs.

86. The method of any one of claims 81-84, wherein one or more of the nucleic acids of the library are RNAs.

87. The method of claim 86, wherein the RNAs are mRNAs.

88. The method of any one of claims 1-87, wherein the tumor antigen is present in a tumor in the subject.

89. The method of any one of claims 1-88, wherein the tumor antigen is a personal tumor antigen that is not present in a second subject.

90. The method of any one of claims 1-88, wherein the tumor antigen is a shared tumor antigen that is present in a second subject.

91. The method of any one of claims 1-90, wherein the tumor antigen is not present in a healthy cell in the subject or is present at a lower level in the healthy cell than in a tumor cell of the subject.

92. The method of any one of claims 1-91, wherein the method induces the production of one or more inflammatory cytokines in the subject.

93. The method of claim 92, wherein the one or more inflammatory cytokines are selected from the group consisting of GM-CSF, MIP-1α, IL-1β, IL-6, IL-8, IL-12, IL-17, IL-18, IP-10, IFN-γ, and TNF-α.

94. The method of any one of claims 1-93, wherein antibodies specific to one or more tumor antigens are generated in the subject.

95. The method of any one of claims 1-94, wherein T cells specific to one or more tumor antigens are generated in the subject.

96. The method of claim 95, wherein the T cells are CD4+ T cells, CD8+ T cells, CD4+CD8+ T cells, or NK-T cells.

97. The method of claim 95 or 96, wherein the T cells are CD8+ T cells.

98. The method of claim 97, wherein the T cells are IFN-γ+CD8+ T cells.

99. The method of claim 95 or 96, wherein the T cells are CD4+ T cells.

100. The method of claim 99, wherein the T cells are IFN-γ+CD4+ T cells.

101. The method of claim 95 or 96, wherein the T cells are NK-T cells.

102. The method of any one of claims 1-101, wherein the method results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of the mass of one or more tumors in the subject.

103. The method of claim 102, wherein the method results in a reduction of at least 30% of the mass of one or more tumors in the subject.

104. The method of any one of claims 1-103, wherein the method results in in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of the mass of a primary tumor in the subject.

105. The method of claim 104, wherein the method results in a reduction of at least 30% of the mass of a primary tumor in the subject.

106. The method of any one of claims 1-105, wherein the method results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of volume of one or more tumors in the subject.

107. The method of claim 106, wherein the method results in a reduction of at least 30% of the volume of one or more tumors in the subject.

108. The method of any one of claims 1-107, wherein the method results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of volume of a primary tumor in the subject.

109. The method of claim 108, wherein the method results in a reduction of at least 30% of the volume of a primary tumor in the subject.

110. The method of any one of claims 1-109, wherein the method results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of a growth rate of one or more tumors in the subject.

111. The method of claim 110, wherein the method results in a reduction of at least 30% of the growth rate of one or more tumors in the subject.

112. The method of any one of claims 1-111, wherein the method results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of a number of circulating cancer cells in the subject.

113. The method of claim 112, wherein the method results in a reduction of at least 30% of a number of circulating cancer cells in the subject.

114. The method of any one of claims 1-113, wherein the method results in a reduction of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% of a number of tumor cells in a tissue in the subject.

115. The method of claim 114, wherein the method results in a reduction of at least 30% of a number of tumor cells in a tissue in the subject.

116. The method of any one of claims 1-115, wherein the method prevents metastasis in the subject for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, at least 24 months, at least 30 months, at least 36 months, at least 42 months, at least 48 months, at least 54 months, at least 60 months, at least 66 months, at least 72 months, at least 78 months, at least 84 months, at least 90 months, at least 96 months, at least 102 months, at least 108 months, at least 114 months, or at least 120 months.

117. The method of any one of claims 1-116, wherein the method prevents the recurrence of cancer for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, at least 24 months, at least 30 months, at least 36 months, at least 42 months, at least 48 months, at least 54 months, at least 60 months, at least 66 months, at least 72 months, at least 78 months, at least 84 months, at least 90 months, at least 96 months, at least 102 months, at least 108 months, at least 114 months, or at least 120 months.

118. The method of claim 117, wherein the method prevents the recurrence of cancer for at least 12 months.

119. The method of any one of claims 1-118, wherein the method further comprises administering an anti-cancer agent to the subject.

120. The method of claim 119, wherein the anti-cancer agent is an antibody.

121. The method of claim 120, wherein the antibody is an immune checkpoint inhibitor.

122. The method of claim 120 or 121, wherein the antibody is an anti-PD-1 antibody, anti-PD-L1 antibody, anti-CTLA-4 antibody, anti-TIGIT antibody, anti-TIM3 antibody, anti-LAG3 antibody, anti-CD200 antibody, or anti-CD200R antibody.

123. The method of claim 122, wherein the anti-cancer agent is a chemotherapeutic agent.

124. The method of claim 119 or 120, wherein the anti-cancer agent is an agonist of a positive immune checkpoint.

125. The method of claim 124, wherein the positive immune checkpoint is selected from the group consisting of CD27, CD28, CD40, CD122, 4-1BB, OX40, GITR, and ICOS.

126. The method of claim 125, wherein the positive immune checkpoint is CD40, 4-1BB, or GITR.

127. The method of any one of claims 1-126, wherein the method further comprises administering FLT3L to the subject systemically or at or near the tumor avatar.

128. The method of claim 127, wherein the FLT3L comprises an amino acid sequence with at least 90%, at least 95%, at least 96%, at least 97%, at least 98%, at least 99%, or up to 100% sequence identity to the amino acid sequence of SEQ ID NO: 30.

129. The method of claim 127 or 128, wherein the FLT3L is administered prior to the generation of the tumor avatar in the subject.

130. The method of any one of claims 1-129, wherein the method further comprises administered a Notch ligand or nucleic acid encoding a Notch ligand to the subject at or near the tumor avatar.

131. The method of claim 130, wherein the Notch ligand is Delta-like ligand 1 (Dll1).

132. The method of any one of claims 1-131, wherein the subject is a human.

133. The method of any one of claims 1-131, wherein the subject is a mouse, rat, hamster, guinea pig, rabbit, dog, cat, bovine, swine, goat, sheep, llama, or alpaca.

134. The method of any one of claims 1-133, wherein the subject has or is at risk of developing cancer.

135. The method of claim 134, wherein the cancer is selected from the group consisting of melanoma, squamous cell cancer, small-cell lung cancer, non-small cell lung cancer, lung cell adenocarcinoma, squamous lung cell carcinoma, peritoneal cancer, hepatocellular cancer, gastrointestinal cancer, esophageal cancer, stomach cancer, pancreatic cancer, glioblastoma, cervical cancer, ovarian cancer, liver cancer, bladder cancer, hepatoma, breast cancer, colon cancer, colorectal cancer, endometrial carcinoma, uterine carcinoma, salivary gland carcinoma, kidney cancer, prostate cancer, vulval cancer, thyroid cancer, hepatic carcinoma, gastric cancer, head-and-neck cancer, leukemia, and lymphoma.

136. A composition comprising:
(i) a tumor antigen, a nucleic acid encoding a tumor antigen, and/or a tumor antigen-containing cell; and
(ii) a hyperactivating agent or a nucleic acid encoding a hyperactivating agent, wherein the hyperactivating agent is capable of inducing secretion of IL-1β, and IL-18 by dendritic cells.

137. The composition of claim 136, wherein the hyperactivating agent is an oxidized phospholipid, a LysM-containing protein, an HMGB1, a granzyme, a cytokine-containing protein, a Toll-like receptor-stimulating protein, a virus, or a virus-like particle.

138. The composition of claim 136 or 137, wherein the hyperactivating agent is an oxidized phospholipid.

139. The composition of claim 136 or 137, wherein the hyperactivating agent is a LysM-containing protein.

140. The composition of claim 136 or 137, wherein the hyperactivating agent is an HMGB1.

141. The composition of claim 136 or 137, wherein the hyperactivating agent is a granzyme.

142. The composition of claim 136 or 137, wherein the hyperactivating agent is a fusion protein comprising a Toll-like receptor-stimulating domain and a glucose-regulated protein (grp170) domain.

143. The composition of claim 136 137, wherein the hyperactivating agent is a virus or virus-like particle.

144. The composition of claim 136 or 137, wherein the hyperactivating agent is a vaccinia or modified vaccinia Ankara (MVA) virus or virus-like particle.

145. The composition of claim 143 or 144, wherein the virus comprises a nucleic acid encoding an engineered receptor, whereby the engineered receptor is expressed on the surface of a cell infected by the virus, wherein the engineered receptor is capable of binding a tumor antigen.

146. The composition of claim 145, wherein the engineered receptor further comprises an intracellular domain of CLEC9A.

147. An engineered receptor comprising:
(i) an extracellular domain that is capable of binding to a tumor antigen;
(ii) a transmembrane domain; and
(iii) an intracellular domain of CLEC9A.

148. The composition of claim 146 or the engineered receptor of claim 140, wherein the intracellular domain of CLEC9A comprises an amino acid sequence with at least 90%, at least 95%, at least 96%, at least 97%, at least 98%, at least 99%, or up to 100% sequence identity to the amino acid sequence of SEQ ID NO: 4.

149. The composition or engineered receptor of claim 148, wherein the intracellular domain of CLEC9A comprises the amino acid sequence of SEQ ID NO: 4.

150. The composition of claim 136 or 137, wherein the hyperactivating agent is a fusion protein comprising a cytokine domain and an anchoring domain, wherein the cytokine domain comprises an amino acid sequence selected from the group consisting of GM-CSF, MIP-1α, IL-1β, IL-6, IL-8, IL-12, IL-17, IL-18, IP-10, IFN-γ, and TNF-α.

151. A fusion protein comprising a cytokine domain and an anchoring domain, wherein the cytokine domain comprises an amino acid sequence selected from the group consisting of GM-CSF, MIP-1α, IL-1β, IL-6, IL-8, IL-12, IL-17, IL-18, IP-10, IFN-γ, and TNF-α.

152. The composition of claim 150 or the fusion protein of claim 151, wherein the anchoring domain is an antibody or antigen-binding fragment thereof.

153. The composition or fusion protein of claim 152, wherein the antibody or antigen-binding fragment thereof binds to an antigen selected from the group consisting of fibronectin extra domain A, fibronectin extra domain B, collagen, and elastin.

154. The method of claim 152, wherein the antibody or antigen-binding fragment thereof binds to a cell adhesion molecule.

155. The composition or fusion protein of claim 153, wherein the anchoring domain is a collagen-binding domain.

156. The composition or fusion protein of claim 155, wherein the anchoring domain is a lumican domain or an A3 domain of von Willebrand Factor.

157. The composition or fusion protein of claim 156, wherein the lumican domain comprises at least 90%, at least 95%, at least 96%, at least 97%, at least 98%, at least 99%, or up to 100% sequence identity to the amino acid sequence of SEQ ID NO: 26.

158. The composition or fusion protein of any one of claims 150-157, wherein the cytokine domain is an IL-1β domain comprising at least 90%, at least 95%, at least 96%, at least 97%, at least 98%, at least 99%, or up to 100% sequence identity to the amino acid sequence of SEQ ID NO: 24.

159. The composition of any one of claims 136-158, further comprising FLT3L.

160. The composition of any one of claims 136-159, further comprising an invariant natural killer T (iNK-T) cell, wherein the iNK-T cell comprises a chimeric antigen receptor (CAR) comprising:
an antigen-binding domain that specifically binds to the tumor antigen;
a transmembrane domain; and
one or more intracellular signaling domains.

161. The composition of claim 160, wherein the one or more intracellular signaling domains are selected from the group consisting of 4-1BB, CD28, ICOS, and CD3ζ.

162. The composition of any one of claims 136-161, wherein the tumor antigen-containing cell comprises CD1d bound to an antigen capable of being recognized by an invariant natural killer T (iNK-T) cell.

163. The composition of claim 162, wherein the antigen capable of being recognized by the iNK-T cell is selected from the group consisting of α-galactosylceramide (αGalCer), α-galactosyldiacylglycerol (αGalDag), α-glucuronsylceramide (α-GLcACer), α-galacturonosylceramide (α-GalACer), phosphatidyl-myo-inositol mannoside (PIM2), β-glucosylceramide (βGlcCer), plasmalogen lysophosphatidylethanolamine (plasmalogen lysoPE), isoglobotrihexosylceramide (iGb3), and α-glucosyldiacyglycerol (αGlcDAG).

164. The composition of any one of claims 136-163, wherein the tumor antigen-containing cell is a 786-O, A549, ACHN, BCP-1, BOSC23, BT-20, BT-549, BxPC-3, Caco-2, Caki-1, Calu-3, CCRF-CEM, DAOY, DU145, H295R, H1299, HaCaT, HAP1, HCC-2998, HCT116, HEK293, HEL, HeLa, HepG2, HL-60, Hs 578T, HT-29, HT1080, Huh7, HUVEC, Jurkat, JY, K562, KBM-7, KM12, LAPC4, LNCaP, LOX-IMVI, M14, MCF-7, MCF-7/ADR-RES, MDA-MB-231, MDA-MB-435, MDA-MB-453, MDA-MB-468, MDA-N, MIA PaCa-2,MOLT-4, MRC-5, NCI-60, NCI-H23, NCI-H226, NCI-H460, NCI/ADR-RES, NK-92, NTERA-2, OVCAR-3, OVCAR-8, PANC-1, PC3, Raji, RPMI-7951, Saos-2, SF-268, SF-539, SH-SY5Y, SK-MEL-5, SK-MEL-28, SK-OV-3, SKBR3, SNB-19, SW-620, T-47D, T98G, TF-1, THP-1, U87, U251, U937, VCaP, VG-1, WI-38, and ZR-75-1 cell.

165. The composition of any one of claims 136-164, further comprising an anti-cancer agent.

166. The composition of any one of claims 136-165, wherein the nucleic acid encoding the tumor antigen encodes more than one tumor antigen.

167. The composition of any one of claims 136-166, wherein the nucleic acid encoding the tumor antigen is comprised in a nucleic acid library that is administered to the subject, wherein each nucleic acid of the library encodes one or more tumor antigens.

168. The composition of any one of claims 136-167, wherein one or more nucleic acids are formulated in a lipid nanoparticle.

169. The composition of any one of claims 136-167, one or more nucleic acids are comprised in a viral vector.

170. The composition of claim 169, wherein the viral vector is an adenovirus, adeno-associated virus, retrovirus, lentivirus, herpesvirus, or poxvirus.

171. A pharmaceutical composition comprising the composition of any one of claims 136-163, and a pharmaceutically acceptable excipient.

172. A kit comprising the composition of any one of claims 136-171, and a cytotoxic agent.

173. The kit of claim 172, wherein the cytotoxic agent is squaric acid, epirubicin, bleomycin, bortezomib, cyclophosphamide, doxorubicin, idarubicin, mitoxantrone, or oxaliplatin.

174. A kit comprising the composition of any one of claims 136-173, and a sensitizing agent.

175. The kit of claim 174, wherein the sensitizing agent is a photosensitizing agent.

176. The kit of claim 175, further comprising a light source.

177. The kit of claim 174, wherein the sensitizing agent is an acoustic sensitizing agent.

178. The kit of claim 177, further comprising an ultrasound source.

179. The kit of any one of claims 172-178, further comprising a delivery device.

180. The kit of claim 179, wherein the delivery device is a needle or jet injector.

181. The composition of any one of claims 136-171, or the kit of any one of claims 165-173, for use in generating an immune response to a tumor antigen.

182. The composition of any one of claims 136-171, or the kit of any one of claims 165-173, for use in treating cancer.

183. The composition of any one of claims 136-171, or the kit of any one of claims 165-173, for use in treating melanoma, squamous cell cancer, small-cell lung cancer, non-small cell lung cancer, lung cell adenocarcinoma, squamous lung cell carcinoma, peritoneal cancer, hepatocellular cancer, gastrointestinal cancer, esophageal cancer, stomach cancer, pancreatic cancer, glioblastoma, cervical cancer, ovarian cancer, liver cancer, bladder cancer, hepatoma, breast cancer, colon cancer, colorectal cancer, endometrial carcinoma, uterine carcinoma, salivary gland carcinoma, kidney cancer, prostate cancer, vulval cancer, thyroid cancer, hepatic carcinoma, gastric cancer, head-and-neck cancer, leukemia, and lymphoma.

184. A method of eliciting an immune response to a tumor antigen in a subject, the method comprising:
(i) contacting a dendritic cell with an oxidized phospholipid;
(ii) contacting the dendritic cell with a tumor cell lysate or a cell comprising one or more tumor antigens; and
(iii) after the contacting of (ii), administering the dendritic cell to a subject at a site that is separate from a tumor in the subject.

185. The method of claim 184, wherein the oxidized phospholipid is an oxidation product of 1-palmitoyl-2-arachidonyl-sn-glycero-3-phosphorylcholine (PAPC).

186. A method of eliciting an immune response to a tumor antigen in a subject, the method comprising:
(i) contacting a cell with a nucleic acid encoding one or more tumor antigens;
(ii) contacting the cell with a CD1d-restricted lipid antigen; and
(iii) after the contactings of (i) and (ii), administering the cell to a subject at a site that is separate from a tumor in the subject.

187. The method of claim 186, wherein:
(a) the cell expresses CD1d on the cell surface; and/or
(b) the method further comprises contacting the cell with a nucleic acid encoding CD1d, prior to the contacting with the CD1d-restricted lipid antigen.

188. The method of claim 186 or 187, wherein the cell is an HEK293 or K562 cell.


SEQUENCE LISTING (31 sequences)

SEQ ID NO: 1 - protein, 4 aa, synthetic construct
  REGION 1..4: note = Synthetic
GGGS

SEQ ID NO: 2 - protein, 7 aa, synthetic construct
  REGION 1..7: note = Synthetic
GGGSGGG

SEQ ID NO: 3 - skipped

SEQ ID NO: 4 - protein, 29 aa, synthetic construct
  REGION 1..29: note = Synthetic
MHEEEIYTSLQWDSPAPDTYQKCLSSNKC

SEQ ID NO: 5 - protein, 51 aa, synthetic construct
  REGION 1..51: note = Synthetic
STVVVEAGDTLWGIAQSKGTTVDAIKKANNLTTDKIVPGQKLQVNNENAAE

SEQ ID NO: 6 - DNA, 1434 bp, Listeria monocytogenes
  source 1..1434: mol_type = genomic DNA
atgaatatgaaaaaagcaactatcgcggctacagctgggattgcggtaacagcatttgctgctccaacaatcgcatccgcaagcactgtagtagttgaagctggtgatactctttggggtatcgcacaaagtaaagggactactgttgacgcaattaaaaaagcaaacaatttaacaacagataaaatcgtaccaggtcaaaaattacaagtaaatgaggttgctgctgctgaaaaaacagagaaatctgttagcgcaacttggttaaacgttcgtagtggcgctggtgttgataacagtattattacgtcaatcaaaggcggaacaaaagtaactgttgaatcaaccgaatctaatggctggaacaaaattacttacaacgacggggaaactggtttcgttaacggtaaatacttaactgacaaagtagcaagcactcctgttgcaccaacacaagaagtgaaaaaagaaactactattcaacaagctgcacctgctgcagaaacaaaaactgaagtaaaacaaactacacaagcaactacacctgcacctaaagtagcagaaacgaaagaaactccagtggtagatcaaaatgctactacacacgctgttaaaagcggtgacactatttgggctttatccgtaaaatacggtgtttccgttcaagacattatgtcatggaataatttatcttcttcttctatttatgtaggtcaaaaacttgctattaaacaaacagccaacacagttactccaaaagcagaagtgaaaaaagaagctccagcagctgaaaaacaagctgcaccagtagttaaagaaaatactaacactaacacaaatactactacagagaaaaaagaaacaacacaacaacaaacagcacctaaagcaccaacagaagctgcaaaaccagctcccgcaccatctacaaacacaaatgctaataaaacgaatacgaatacaaacaatactaatacaagtacaccatctaaaaataccaatactaatacaaactccaatacgaatacaaactcaaatacgaatgctaatcaaggttcttctaacaataacagcaattcaagtgcaagtgctattattgctgaagctcaaaaacaccttggaaaagcttattcatggggtggtaacggaccaactacatttgattgctctggttacactaaatatgtatttgctaaagcgggaatctcccttccacgtacttctggcgcacaatacgctagcactacaagaatctctgaatctcaagcaaaacctggtgatttagtattctttgactatggtagcggaatttctcacgttggtatctacgttggtaatggtcaaatgattaacgcgcaagacaatggcgttaaatacgataacatccacggctctggctggggtaaatatctagttggcttcggtcgcgtataa

SEQ ID NO: 7 - protein, 477 aa, Listeria monocytogenes
MNMKKATIAATAGIAVTAFAAPTIASASTVVVEAGDTLWGIAQSKGTTVDAIKKANNLTTDKIVPGQKLQVNEVAAAEKTEKSVSATWLNVRSGAGVDNSIITSIKGGTKVTVESTESNGWNKITYNDGETGFVNGKYLTDKVASTPVAPTQEVKKETTIQQAAPAAETKTEVKQTTQATTPAPKVAETKETPVVDQNATTHAVKSGDTIWALSVKYGVSVQDIMSWNNLSSSSIYVGQKLAIKQTANTVTPKAEVKKEAPAAEKQAAPVVKENTNTNTNTTTEKKETTQQQTAPKAPTEAAKPAPAPSTNTNANKTNTNTNNTNTSTPSKNTNTNTNSNTNTNSNTNANQGSSNNNSNSSASAIIAEAQKHLGKAYSWGGNGPTTFDCSGYTKYVFAKAGISLPRTSGAQYASTTRISESQAKPGDLVFFDYGSGISHVGIYVGNGQMINAQDNGVKYDNIHGSGWGKYLVGFGRV

SEQ ID NO: 8 - protein, 154 aa, synthetic construct
  REGION 1..154: note = Synthetic
MPNSEPASLLELFNSIATQGELVRSLKAGNASKDEIDSAVKMLVSLKMSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKK

SEQ ID NO: 9 - protein, 123 aa, synthetic construct
  REGION 1..123: note = Synthetic
ARQNHLFEQYREKRPEAAQLLEDVQAALKPFSVKLNETTDPDKKQMLERIQHAVQLATEPLEKAVQSRLTGEEVNSCVEVLLEEAKDLLSDWLDSTLGCDVTDNSIFSKLPKFWEGDFHRDME

SEQ ID NO: 10 - protein, 172 aa, synthetic construct
  REGION 1..172: note = Synthetic
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDT

SEQ ID NO: 11 - protein, 215 aa, synthetic construct
  REGION 1..215: note = Synthetic
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 12 - protein, 262 aa, Homo sapiens
MRNSYRFLASSLSVVVSLLLIPEDVCEKIIGGNEVTPHSRPYMVLLSLDRKTICAGALIAKDWVLTAAHCNLNKRSQVILGAHSITREEPTKQIMLVKKEFPYPCYDPATREGDLKLLQLMEKAKINKYVTILHLPKKGDDVKPGTMCQVAGWGRTHNSASWSDTLREVNITIIDRKVCNDRNHYNFNPVIGMNMVCAGSLRGGRDSCNGDSGSPLLCEGVFRGVTSFGLENKCGDPRGPGVYILLSKKHLNWIIMTIKGAV

SEQ ID NO: 13 - DNA, 1488 bp, Salmonella enterica
  source 1..1488: mol_type = genomic DNA
atggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtcaggcgattgctaaccgttttaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggtgccaacgacggtgaaactatcgatatcgatctgaagcagatcaactctcagaccctgggtctggatacgctgaatgtgcaacaaaaatataaggtcagcgatacggctgcaactgttacaggatatgccgatactacgattgctttagacaatagtacttttaaagcctcggctactggtcttggtggtactgaccagaaaattgatggcgatttaaaatttgatgatacgactggaaaatattacgccaaagttaccgttacggggggaactggtaaagatggctattatgaagtttccgttgataagacgaacggtgaggtgactcttgctggcggtgcgacttccccgcttacaggtggactacctgcgacagcaactgaggatgtgaaaaatgtacaagttgcaaatgctgatttgacagaggctaaagccgcattgacagcagcaggtgttaccggcacagcatctgttgttaagatgtcttatactgataataacggtaaaactattgatggtggtttagcagttaaggtaggcgatgattactattctgcaactcaaaataaagatggttccataagtattaatactacgaaatacactgcagatgacggtacatccaaaactgcactaaacaaactgggtggcgcagacggcaaaaccgaagttgtttctattggtggtaaaacttacgctgcaagtaaagccgaaggtcacaactttaaagcacagcctgatctggcggaagcggctgctacaaccaccgaaaacccgctgcagaaaattgatgctgctttggcacaggttgacacgttacgttctgacctgggtgcggtacagaaccgtttcaactccgctattaccaacctgggcaacaccgtaaacaacctgacttctgcccgtagccgtatcgaagattccgactacgcgaccgaagtttccaacatgtctcgcgcgcagattctgcagcaggccggtacctccgttctggcgcaggcgaaccaggttccgcaaaacgtcctctctttactgcgttaa

SEQ ID NO: 14 - protein, 495 aa, Salmonella enterica [sequence omitted]

SEQ ID NO: 15 - DNA, 3000 bp, Mus musculus
  source 1..3000: mol_type = genomic DNA
atggcagctacagtaaggaggcagaggccaaggaggctactctgttgggccttggtggctgtcctcttggcagacctgttggcactgagcgacacattggctgtgatgtctgtagacctgggcagtgaatccatgaaggtggccattgtcaagcctggagtgcccatggagattgtattgaacaaggaatctcggaggaaaactccagtgactgtgaccttgaaagaaaatgaaaggtttttaggtgatagtgcagccggcatggccatcaagaacccaaaggctacgctccgttatttccagcacctccttggaaaacaggcggataaccctcatgtggccctttaccggtcccgtttcccagaacatgagctaattgttgacccacagaggcagactgtgcgcttccagatcagtccgcagctgcagttctctcccgaggaggtactgggcatggttctgaactactcccgttccttggctgaagattttgctgaacaacccattaaggatgcagtgatcaccgtgccagcctttttcaaccaggctgagcgccgagctgtgctgcaggctgctcggatggctggcctcaaggtgctgcagctcatcaatgacaacactgccacagccctcagctacggtgtcttccgccggaaagatatcaattccactgcacagaacgtcatgttctatgacatgggctcgggcagcactgtgtgcaccatcgtcacctaccagacagtgaagactaaggaggctgggatgcaaccacagctgcagatccggggcgtgggatttgaccgcaccctgggtggcctggagatggagcttcggcttcgagaacacctggctaagctcttcaatgagcagcgcaagggccagaaagccaaggatgttcgggaaaacccccgggccatggccaaactgcttcgggaagccaaccggcttaaaaccgtcctgagtgccaacgctgatcacatggcacagattgagggcttgatggatgatgtggacttcaaggccaaagtaactcgagtggaattcgaggagctgtgtgcagatttgtttgaccgtgtgcctggacctgtgcagcaggccttgcagagtgcagagatgagcttggatcaaattgagcaggtgatcctggtgggcggggccactcgtgttcccaaagttcaagaagtgctgctcaaggccgtgggcaaggaggaactaggaaagaacatcaatgcggacgaagctgctgccatgggggctgtgtaccaggcagcggcgctcagcaaggccttcaaagtgaagccatttgttgtgcgggatgctgtcatttacccaatcctggtggagttcacaagggaggtggaggaggagcctgggctccgaagcctgaaacacaataagcgtgtgctcttctcccgaatggggccctaccctcagcgcaaagtcatcacctttaaccgctacagccatgatttcaacttccacatcaactacggtgacctgggcttcctggggcctgaggatcttcgggtatttggctcccagaatctgaccacagtaaaactaaaaggcgtgggagagagcttcaagaaatatcccgactatgagtccaaaggcatcaaggcccactttaacctggatgagagtggcgtgctcagtttagacagggtggagtccgtatttgagaccctggtggaggatagcccagaggaagaatctactcttaccaaacttggcaacaccatatccagcctgtttggaggtggtacctcatcagatgccaaagagaatggtactgatgctgtacaggaggaagaggagagccccgctgaggggagcaaggatgagcctgcagagcagggggaactcaaggaagaagctgaacccccagcagaggagacctctcagcctccaccctctgagcctaagggggatgcagcccgtgagggagagaaacctgatgaaaaagagagtggggacaagcctgaggcccagaagcccaatgagaaggggcaagcagggcctgagggtgctgctccagctcctgaggaggacaaaaagccgaaacctgcccggaagcagaaaatggtggaggagataggtgtggagctggctgtcttggacctgcctgacttgccagaggatgagctggcccgttctgtgcagaaacttgaagaactgaccctgcgcgacctagagaagcaggagagggagaaagctgccaacagcttggaggctttcatctttgagacccaggacaagctgtaccagcctgagtaccaggaagtgtccactgaggaacagcgggaggagatctcggggaaactcagcgccacttctacctggctggaggatgagggatttggagccaccactgtgatgctgaaggacaagctggctgagctgagaaagctgtgccaagggctgttttttcgggtggaagaacgcaggaaatggccagagcggctttcagctctggataatctcctcaaccattccagcattttcctcaagggtgcccggctcatcccggagatggaccaggtcttcactgaagtggagatgacgacattagagaaagttatcaatgacacctgggcctggaagaatgcaactctggccgaacaagccaagcttcctgccacagagaagcctgtgctgctttcaaaagacattgaggccaaaatgatggccctggaccgggaggtacagtatctactcaataaggccaagtttaccaagccacggccacggcccaaagacaagaatggcacccgggcagaacctcccctcaatgccagtgctggtgaccaagaggagaaggtcattccacctgcaggccagactgaagaggcgaaacccattttagaacctgacaaagaagagactggtacggaaccagcagactcggagcctctggaattaggaggtcctggagctggacctgaacaggaagagcagtcagcaggacagaagcggccttcaaagaacgatgaactataa

SEQ ID NO: 16 - protein, 999 aa, Homo sapiens
MADKVRRQRPRRRVCWALVAVLLADLLALSDTLAVMSVDLGSESMKVAIVKPGVPMEIVLNKESRRKTPVIVTLKENERFFGDSAASMAIKNPKATLRYFQHLLGKQADNPHVALYQARFPEHELTFDPQRQTVHFQISSQLQFSPEEVLGMVLNYSRSLAEDFAEQPIKDAVITVPVFFNQAERRAVLQAARMAGLKVLQLINDNTATALSYGVFRRKDINTTAQNIMFYDMGSGSTVCTIVTYQMVKTKEAGMQPQLQIRGVGFDRTLGGLEMELRLRERLAGLFNEQRKGQRAKDVRENPRAMAKLLREANRLKTVLSANADHMAQIEGLMDDVDFKAKVTRVEFEELCADLFERVPGPVQQALQSAEMSLDEIEQVILVGGATRVPRVQEVLLKAVGKEELGKNINADEAAAMGAVYQAAALSKAFKVKPFVVRDAVVYPILVEFTREVEEEPGIHSLKHNKRVLFSRMGPYPQRKVITFNRYSHDFNFHINYGDLGFLGPEDLRVFGSQNLTTVKLKGVGDSFKKYPDYESKGIKAHFNLDESGVLSLDRVESVFETLVEDSAEEESTLTKLGNTISSLFGGGTTPDAKENGTDTVQEEEESPAEGSKDEPGEQVELKEEAEAPVEDGSQPPPPEPKGDATPEGEKATEKENGDKSEAQKPSEKAEAGPEGVAPAPEGEKKQKPARKRRMVEEIGVELVVLDLPDLPEDKLAQSVQKLQDLTLRDLEKQEREKAANSLEAFIFETQDKLYQPEYQEVSTEEQREEISGKLSAASTWLEDEGVGATTVMLKEKLAELRKLCQGLFFRVEERKKWPERLSALDNLLNHSSMFLKGARLIPEMDQIFTEVEMTTLEKVINETWAWKNATLAEQAKLPATEKPVLLSKDIEAKMMALDREVQYLLNKAKFTKPRPRPKDKNGTRAEPPLNASASDQGEKVIPPAGQTEDAEPISEPEKVETGSEPGDTEPLELGGPGAEPEQKEQSTGQKRPLKNDEL

SEQ ID NO: 17 - protein, 94 aa, synthetic construct
  REGION 1..94: note = Synthetic
ATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 18 - protein, 564 aa, synthetic construct
  REGION 1..564: note = Synthetic
KVKPFVVRDAVVYPILVEFTREVEEEPGIHSLKHNKRVLFSRMGPYPQRKVITFNRYSHDFNFHINYGDLGFLGPEDLRVFGSQNLTTVKLKGVGDSFKKYPDYESKGIKAHFNLDESGVLSLDRVESVFETLVEDSAEEESTLTKLGNTISSLFGGGTTPDAKENGTDTVQEEEESPAEGSKDEPGEQVELKEEAEAPVEDGSQPPPPEPKGDATPEGEKATEKENGDKSEAQKPSEKAEAGPEGVAPAPEGEKKQKPARKRRMVEEIGVELVVLDLPDLPEDKLAQSVQKLQDLTLRDLEKQEREKAANSLEAFIFETQDKLYQPEYQEVSTEEQREEISGKLSAASTWLEDEGVGATTVMLKEKLAELRKLCQGLFFRVEERKKWPERLSALDNLLNHSSMFLKGARLIPEMDQIFTEVEMTTLEKVINETWAWKNATLAEQAKLPATEKPVLLSKDIEAKMMALDREVQYLLNKAKFTKPRPRPKDKNGTRAEPPLNASASDQGEKVIPPAGQTEDAEPISEPEKVETGSEPGDTEPLELGGPGAEPEQKEQSTGQKRPL

SEQ ID NO: 19 - DNA, 3481 bp, synthetic construct
  misc_feature 1..3481: note = Synthetic
  source 1..3481: mol_type = other DNA
tattaaaatcttaataggttttgataaaagcgaacgtggggaaacccgaaccaaaccttcttctaaattctctctcatctctcttaaagcaaacttctctcttgtctttcttgcatgagcgatcttcaacgttgtcagatcgtgcttcggcaccagtacaatgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgaaatggaaagcattatgagccgtggtattccttcaggaattttggaggaaaaagctattcagttcaaacgtgccaaagaagggaataaacccttgaaggatgagattcccaagcctgaggatatgtatgtgtctcacacttctaaatggaatgtgctcagaaaaatgagccaaaagactgtggatctttccaaagcagctgctgggatgggattcatcaataagcatatgcttacgggcaacatcttggcacaaccaacaacagtcttggatattcccgtcacaaaggataaaacacttgcgatggccagtgattttattcgtaaggagaatctcaagacttctgccattcacattggagcaattgagattattatccagagctttgcttcccctgaaagtgatttgatgggaggctttttgcttgtggattctttacacactgatacagctaatgctattcgtagcatttttgttgctccaatgcggggaggaagaccagtcagagtggtgaccttcccaaatacactggcacctgtatcatgtgatctgaacaatagattcaagctcatttgctcattgccaaactgtgatattgtccagggtagccaagtagcagaagtgagtgtaaatgttgcaggatgtgctacttccatagagaaatctcacaccccttcccaattgtatacagaggaatttgaaaaggagggtgctgttgttgtagaatacttaggcagacagacctattgtgctcagcctagcaatttacccacagaagaaaaacttcggtcccttaagtttgactttcatgttgaacaaccaagtgtcctgaagttatccaattcctgcaatgcgcactttgtcaagggagaaagtttgaaatactctatttctggcaaagaagcagaaaaccatgcagttcatgctactgtggtctctcgagaaggggcttctgcggcacccaagcaatatgatcctattttgggacgggtgctggatccacgaaatgggaatgtggcttttccacaaatggagcaaaacttgtttgccctttctttggatgatacaagctcagttcgtggttctttgcttgacacaaaattcgcacaaactcgagttttgttgtccaaggctatggctggtggtgatgtgttattggatgagtatctctatgatgtggtcaatggacaagattttagagctactgtcgcttttttgcgcacccatgttataacaggcaaaataaaggtgacagctaccaccaacatttctgacaactcgggttgttgtttgatgttggccataaatagtggtgtgaggggtaagtatagtactgatgtttatactatctgctctcaagactccatgacgtggaacccagggtgcaaaaagaacttctcgttcacatttaatccaaacccttgtggggattcttggtctgctgagatgataagtcgaagcagagttaggatgacagttatttgtgtttcgggatggaccttatctcctaccacagatgtgattgccaagctagactggtcaattgtcaatgagaaatgtgagcccaccatttaccacttggctgattgtcagaattggttaccccttaatcgttggatgggaaaattgacttttccccagggtgtgacaagtgaggttcgaaggatgcctctttctataggaggcggtgctggtgcgactcaagctttcttggccaatatgcccaattcatggatatcaatgtggagatattttagaggtgaacttcactttgaagttactaaaatgagctctccatatattaaagccactgttacatttctcatagcttttggtaatcttagtgatgcctttggtttttatgagagttttcctcatagaattgttcaatttgctgaggttgaggaaaaatgtactttggttttctcccaacaagagtttgtcactgcttggtcaacacaagtaaaccccagaaccacacttgaagcagatggttgtccctacctatatgcaattattcatgatagtacaacaggtacaatctccggagattttaatcttggggtcaagcttgttggcattaaggatttttgtggtataggttctaatccgggtattgatggttcccgcttgcttggagctatagcacaaggacctgtttgtgctgaagcctcagatgtgtatagcccatgtatgatagctagcactcctcctgctccattttcagacgttacagcagtaacttttgacttaatcaacggcaaaataactcctgttggtgatgacaattggaatacgcacatttataatcctccaattatgaatgtcttgcgtactgctgcttggaaatctggaactattcatgttcaacttaatgttaggggtgctggtgtcaaaagagcagattgggatggtcaagtctttgtttacctgcgccagtccatgaaccctgaaagttatgatgcgcggacatttgtgatctcacaacctggttctgccatgttgaacttctcttttgatatcatagggccgaatagcggatttgaatttgccgaaagcccatgggccaatcagaccacctggtatcttgaatgtgttgctaccaatcccagacaaatacagcaatttgaggtcaacatgcgcttcgatcctaatttcagggttgccggcaatatcctgatgcccccatttccactgtcaacggaaactccaccgttattaaagtttaggtttcgggatattgaacgctccaagcgtagtgttatggttggacacactgctactgctgcttaactctggtttcattaaattttctttagtttgaatttactgttatttggtgtgcatttctatgtttggtgagcggttttctgtgctcagagtgtgtttattttatgtaatttaatttctttgtgagctcctgtttagcaggtcgtcccttcagcaaggacacaaaaagattttaattttatt

SEQ ID NO: 20 - protein, 1046 aa, synthetic construct
  REGION 1..1046: note = Synthetic
MFSFTEAKSKISLWTRSAAPLNNVYLSYSCRCGLGKRKLAGGCCSAPYITCYDSADFRRVQYLYFCLTRYCCLYFFLLLLADWFYKKSSIFFETEFSRGFRTWRKIVKLLYILPKFEMESIMSRGIPSGILEEKAIQFKRAKEGNKPLKDEIPKPEDMYVSHTSKWNVLRKMSQKTVDLSKAAAGMGFINKHMLTGNILAQPTTVLDIPVTKDKTLAMASDFIRKENLKTSAIHIGAIEIIIQSFASPESDLMGGFLLVDSLHTDTANAIRSIFVAPMRGGRPVRVVTFPNTLAPVSCDLNNRFKLICSLPNCDIVQGSQVAEVSVNVAGCATSIEKSHTPSQLYTEEFEKEGAVVVEYLGRQTYCAQPSNLPTEEKLRSLKFDFHVEQPSVLKLSNSCNAHFVKGESLKYSISGKEAENHAVHATVVSREGASAAPKQYDPILGRVLDPRNGNVAFPQMEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQGPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPLLKFRFRDIERSKRSVMVGHTATAA

SEQ ID NO: 21 - protein, 189 aa, synthetic construct
  REGION 1..189: note = Synthetic
GPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPL

SEQ ID NO: 22 - protein, 374 aa, synthetic construct
  REGION 1..374: note = Synthetic
MEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQ

SEQ ID NO: 23 - DNA, 810 bp, Homo sapiens
  source 1..810: mol_type = genomic DNA
atggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaa

SEQ ID NO: 24 - protein, 111 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNE

SEQ ID NO: 25 - DNA, 1017 bp, Homo sapiens
  source 1..1017: mol_type = genomic DNA
atgagtctaagtgcatttactctcttcctggcattgattggtggtaccagtggccagtactatgattatgattttcccccatcaatttatgggcaatcatcaccaaactgtgcaccagaatgtaactgccctgaaagctacccaagtgccatgtactgtgatgagctgaaattgaaaagtgtaccaatggtgcctcctggaatcaagtatctttaccttaggaataaccagattgaccatattgatgaaaaggcctttgagaatgtaactgatctgcagtggctcattctagatcacaacgttctagaaaactccaagataaaagggagagttttctctaaattgaaacaactgaagaagctgcatataaaccacaacaacctgacagagtctgtgggcccacttcccaaatctctggaggatctgcagcttactcataacaagatcacaaagctgggctcttttgaaggattggtaaacctgaccttcatccatctccagcacaatcggctgaaagaggatgctgtttcagctgcttttaaaggtcttaaatcactcgaataccttgacttgagcttcaatcagatagccagactgccttctggtctccctgtctctcttctaactctctacttagacaacaataagatcagcaacatccctgatgagtatttcaagcgttttaatgcattgcagtatctgcgtttatctcacaacgaactggctgatagtggaatacctggaaattctttcaatgtgtcatccctggttgagctggatctgtcctataacaagcttaaaaacataccaactgtcaatgaaaaccttgaaaactattacctggaggtcaatcaacttgagaagtttgacataaagagcttctgcaagatcctggggccattatcctactccaagatcaagcatttgcgtttggatggcaatcgcatctcagaaaccagtcttccaccggatatgtatgaatgtctacgtgttgctaacgaagtcactcttaattaa

SEQ ID NO: 26 - protein, 320 aa, synthetic construct
  REGION 1..320: note = Synthetic
QYYDYDIPLFMYGQISPNCAPECNCPHSYPTAMYCDDLKLKSVPMVPPGIKYLYLRNNQIDHIDEKAFENVTDLQWLILDHNLLENSKIKGKVFSKLKQLKKLHINYNNLTESVGPLPKSLQDLQLTNNKISKLGSFDGLVNLTFIYLQHNQLKEDAVSASLKGLKSLEYLDLSFNQMSKLPAGLPTSLLTLYLDNNKISNIPDEYFKRFTGLQYLRLSHNELADSGVPGNSFNISSLLELDLSYNKLKSIPTVNENLENYYLEVNELEKFDVKSFCKILGPLSYSKIKHLRLDGNPLTQSSLPPDMYECLRVANEITVN

SEQ ID NO: 27 - DNA, 8442 bp, Homo sapiens
  source 1..8442: mol_type = genomic DNA
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgcccccccacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgaccgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggtgccaggccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagactcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaaccgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcaggtgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagtga

SEQ ID NO: 28 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 29 - DNA, 708 bp, Homo sapiens
  source 1..708: mol_type = genomic DNA
atgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcggggcgctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgccccccgtccccagtccccaggacctgctgcttgtggagcactga

SEQ ID NO: 30 - protein, 235 aa, Homo sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 31 - protein, 338 aa, Homo sapiens
MSLSAFTLFLALIGGTSGQYYDYDFPLSIYGQSSPNCAPECNCPESYPSAMYCDELKLKSVPMVPPGIKYLYLRNNQIDHIDEKAFENVTDLQWLILDHNLLENSKIKGRVFSKLKQLKKLHINHNNLTESVGPLPKSLEDLQLTHNKITKLGSFEGLVNLTFIHLQHNRLKEDAVSAAFKGLKSLEYLDLSFNQIARLPSGLPVSLLTLYLDNNKISNIPDEYFKRFNALQYLRLSHNELADSGIPGNSFNVSSLVELDLSYNKLKNIPTVNENLENYYLEVNQLEKFDIKSFCKILGPLSYSKIKHLRLDGNRISETSLPPDMYECLRVANEVTLN